(19) United States
(12) Patent Application Publication
(10) Pub. No.: US 2025/0236666 A1
(43) Pub. Date: Jul. 24, 2025

(54) Antibodies Against Extracellular Epitopes of Human TRPV6 Channel and their Diagnostic and Therapeutic Uses

(71) Applicants: Universite de Lille, Lille (FR); INSERM (Institut National de la Santé et de la Recherche Médicale), Paris (FR)

(72) Inventors: Natalia Prevarskaya, Rumegies (FR); V'yacheslav Lehen'Kyi, Villeneuve d'Ascq (FR); Aurélien Haustrate, Halluin (FR)

(73) Assignees: Universite de Lille, Lille (FR); INSERM (Institut National de la Santé et de la Recherche Médicale), Paris (FR)

(21) Appl. No.: 18/700,859

(22) PCT Filed: Oct. 14, 2022
(86) PCT No.: PCT/EP2022/078728

§ 371 Date: Apr. 12, 2024

(30) Foreign Application Priority Data
Oct. 14, 2021 (EP) 21306438.9

(51) Int. Cl. C07K 16/28; A61K 39/00; A61P 35/00

(52) U.S. Cl. CPC C07K 16/28; A61P 35/00; A61K 2039/505; C07K 2317/24; C07K 2317/34; C07K 2317/73; C07K 2317/92

(57) ABSTRACT

The invention relates to antibodies against extracellular epitopes of human Transient Receptor Potential Vanilloid 6 (TRPV6) channel protein, in particular antibodies which modulate TRPV6 channel activity on the plasma membrane and thereby trigger apoptosis of cancer cells expressing TRPV6. The invention relates also to the use of said antibodies for the diagnosis, prognosis and treatment of diseases involving TRPV6 channels, in particular diseases associated with TRPV6-expression such as cancers. The invention further relates to peptide antigens from human TRPV6 protein useful for the production of said antibodies.

### FIELD OF THE INVENTION

The invention relates to antibodies against extracellular epitopes of human Transient Receptor Potential Vanilloid 6 (TRPV6) channel protein, in particular antibodies which modulate TRPV6 channel activity on the plasma membrane and thereby trigger apoptosis of cancer cells expressing TRPV6. The invention relates also to the use of said antibodies for the diagnosis, prognosis and treatment of diseases involving TRPV6 channels, in particular diseases associated with TRPV6expression such as cancers. The invention further relates to peptide antigens from human TRPV6 protein useful for the production of said antibodies.

### BACKGROUND OF THE INVENTION

Transient receptor potential vanilloid subfamily member 6 (TRPV6) is a highly calcium-selective TRP channel which mediates calcium uptake in epithelial tissues and is involved in calcium homeostasis in the body (Clapham et al., Nat. Rev. Neurosci., 2000, 2, 387-96; Hoenderop et al., Pflugers Arch. 2003, 446, 304-8). TRPV6 is also known as Transient receptor potential cation channel subfamily V member 6 (TrpV6), CaT-like (CaT-L); Calcium transport protein 1 (CaT1) or Epithelial calcium channel 2 (ECaC2). TRPV6 has four subunits that form a transmembrane domain (TMD) with a central ion channel pore that is flanked by intracellular N- and C-terminal domains. The TMD is composed of transmembrane helices S1-S6 and a re-entrant pore loop (P-loop) between S5 and S6 (FIG. 1). TRPV6 is glycosylated at a N-glycosylation site situated in the first extracellular loop (position 397 of human TRPV6 sequence; FIG. 1). Human TRPV6 has the amino acid sequence UniProtKB/Swiss-Prot NP_061116.5 or Q9H1D0.3 (SEQ ID NO: 1). TRPV6 3D crystal structure has been determined (Saotome et al., Nature 2016, 534, 506-511).

Altered TRPV6 expression is associated with a variety of human diseases, including cancers. TRPV6 is overexpressed in endometrial cancers, leukemia, and carcinomas of the breast, pancreas, prostate, colon, ovarian, and thyroid (Peng et al., Biochem. Biophys. Res. Commun., 2000, 278, 326-332; Wissenbach et al., J. Biol. Chem., 2001, 276, 19461-19468; Fleet et al., Am. J. Physiol. Gastrointest. Liver Physiol. 2002, 283, G618-G625; Zhuang et al., Lab. Invest., 2002, 82, 1755-1764; Fixemer et al., Oncogene, 2003, 22, 7858-7861; Wissenbach et al., Biochem. Biophys. Res. Commun., 2004, 322, 1359-1363; Taparia et al., Eur. J. Nutr. 2006, 45, 196-204; Wissenbach, U.& Niemeyer, B. A., Handb. Exp. Pharmacol., 2007, 179, 221-234; Bolanz et al., Mol. Cancer Ther., 2008, 7, 271-279; Bolanz et al., Mol. Cancer Res., 2009, 7, 2000-2010; Semenova et al., Am. J. Physiol. Cell. Physiol., 2009, 296, C1098-C1104; Lehen'kyi et al., PLoS ONE, 2011, 6, e16856; Dhennin-Duthille et al., Cell Physiol Biochem., 2011, 28, 813-22; Zheng et al., Biochem. Pharmacol., 2012, 84, 391-401; Bowen et al., PLoS ONE, 2013, 8, e58866; Fecher-Trost et al., Handb. Exp. Pharmacol., 2014, 222, 359-384; Singh et al., Nature communications, 2018, DOI:10.1038; Song et al., Oncol Rep., 2018, 39, 1432-1440; Skrzypski et al., Biosci Rep., 2016, 36, e00372. doi: 10.1042; Masamune et al., Gastroenterology, 2020, 158, 1626-1641). Altered TRPV6 expression is associated with skin diseases such as psoriasis (Cubillos et al., J. Int J. Mol. Med., 2016, 38, 1083-92), alopecia and dermatitis (Bianco S D et al., J. Bone Miner. Res. 2007, 22, 274-85), abnormal epithelial proliferation (Dai W et al., Cell Death Differ. 2014, 21, 568-81), skin aging (Li W et al., J. Gerontol. A Biol. Sci. Med. Sci., 2015, 70, 263-72), skin permeability barrier (Do B H et al., Acta Otolaryngol., 2017, 137, 1039-1045), hyperphosphatemia and ectopic calcification (Jurutka μW et al., J. Bone Miner. Res., 2007, 22 Suppl 2:V2-10), nerve/brain system disorders such as deafness and postpuberty goiter (Wangemann P et al., Am. J. Physiol. Renal Physiol., 2007, 292, F1345-53), preweaning (Lee GSt al., J. Bone Mine.r Res., 2007, 22, 1968-78), nerve excitability (Brittain J M et al., Channels (Austin, Tex.), 2012 March-April; 6(2):94-102), estrous cycle and hypothalamus disorders (Kumar S et al., Neuroscience, 2017, 344, 204-216), circadian rhythm (Yang Q J et al., Drug Metab. Dispos., 2018, 46, 75-87) drug addiction (Janssens A, et al., Pharmacol. Res., 2018, 136, 83-89), Parkinson's disease (Claro da Silva et al., J. Steroid Biochem. Mol. Biol., 2016, 163, 77-87), pain sensation (Jiang Y et al., Onco.l Lett., 2016, 12, 1164-1170), digestive tract disorders like Crohn's disease (Huybers S et al., Inflamm. Bowel Dis., 2008, 14, 803-11), hypercalcemia (Zella L A et al., Endocrinology, 2009, 150, 3448-56), colonic crypt hyperplasia (Peleg S et al., Am. J. Physiol. Gastrointest. Liver Physiol., 2010, 299(3)), intestinal bowel syndrome (Ishizawa M et al., Int. J. Mol. Sci., 2018, 19(7)), Kidney diseases such as calcification of arteries and kidney (Ignat M et al., Proc. Natl. Acad. Sci. USA., 2008, 105, 2598-603), chronic kidney disease (Torremade et al., PLoS One, 2017, 12, e0170654), bone mineral density and osteoporosisdiseases and disorders (Bianco S D et al., J. Bone Miner. Res., 2007, 22, 274-85). Gynecological disorders such astrophoblast disorders (Bernucci L et al, Placenta, 2006, 27, 1082-95), female infertility (Yang H et al., Mol. Reprod. Dev., 2011, 78, 274-82), diabetes mellitus (Lee C T et al., Kidney Int., 2006, 69, 1786-91).

TRPV6 has been implicated in tumor development and progression, and its overexpression pattern correlates with the aggressiveness of the disease (Wissenbach et al., 2001; Fixemer et al., 2003; Wissenbach et al., 2004; Lehen'kyi et al., PLoS ONE, 2011; Peng et al., Biochem. Biophys. Res. Commun., 2001, 282, 729-734; Lehen'kyi et al., Oncogene, 2007, 26, 7380-7385; Lehen'kyi et al., J. Physiol., 2012, 590, 1369-1376).

Ca²⁺ is a critical regulator of cell proliferation, suggesting a role for TRPV6 in the potentiation of calcium-dependent cell proliferation and inhibition of apoptosis (Bowen et al., 2013; Lehen'kyi et al., Am. J. Physiol., 2011, 301, C1281-9; Raphaël et al., Proc. Natl. Acad. Sci. USA., 2014, 111, E3870-9). Modulators of TRPV6 may, therefore, offer a novel therapeutic strategy for treatment of TRPV6-expressing tumors tumors (Bolanz et al., 2008; Bowen et al., 2013; Lehen'kyi et al., 2007; Schwarz, et al., Cell Calcium, 2006, 39, 163-173). Whatever the alteration of TRPV6 activity by the modulator, the effects on the cells will be devastating. Indeed, inhibition of TRPV6 channel activity or expression will decrease the level of calcium required for pro-survival and anti-apoptotic pathways by decreasing calcineurin phosphatase activity (Roderick et al., Nat Rev Cancer., 2008, May; 8(5):361-75). Concerning the activation of the TRPV6 channel, it will increase the calcium uptake in an uncontrolled manner, which will overload the mitochondria and cause the release of cytochrome C triggering the pro-apoptotic cascade, (Bernardi et al., Subcell Biochem 2007, 45, 481-506).

A Small molecule inhibitor and a peptide inhibitor of TRPV6 channel activity have been disclosed (TH-1177, Landowski et al., Pharm Res., 2011, 28, 322-30; soricidin or SOR-C13, Bowen et al., PLoS One. 2013, 8, e58866 and International PCT application WO 2009/114943).

Several anti-TRPV6 polyclonal antibodies designed to recognize intracellular epitopes in the N-terminus or C-terminus have been disclosed and are commercially available (Lehen'kyi et al., 2007; Van der Eerden et al., J. Cell. Physiol., 2011, 227, 1951-1959; TRPV6 #C-16 (Santa Cruz); sc-31445 (Borthwick et al., Cell Calcium, 2008, 44, 147-57; Dhennin-Duthille et al., 2011); #ACC-036 (Alomone). The antibodies are used to detect TRPV6 and study its correlation with cancer.

However, no anti-TRPV6 antibody, in particular anti-TRPV6 monoclonal antibody, able to recognize specifically an extracellular epitope of human TRPV6 channel has been reported so far. In addition, no anti-TRPV6 antibody able to modulate TRPV6 channel activity and thereby inhibit cancer cell proliferation has been reported so far.

### SUMMARY OF THE INVENTION

The inventors have generated antibodies raised against TRPV6 channel extracellular epitopes corresponding to the extracellular loop between S1 and S2 transmembrane domains, and the extracellular part of the pore region (FIG. 1). These antibodies were capable of modulating TRPV6 activity on the plasma membrane changing calcium entry current and impairing calcium signaling thus triggering cancer cell apoptosis in vitro (FIGS. 7 to 11 and 13 to 15). A mouse monoclonal antibody generated against an epitope at the pore level was shown capable of suppressing tumor growth and metastasis appearance in vivo in human tumor xenograft mouse model (FIG. 12). These data open perspectives for the therapeutic use of these anti-TRPV6 antibodies in diseases and disorders where TRPV6 channel is involved, in particular diseases associated with TRPV6-expression such as cancers.

The generated antibodies which are able to detect TRPV6 at the plasma membrane are useful for all in vitro or in vivo detection or diagnosis immunoassays on live, fixed or denatured cells or tissues such as but not limited to immunoblotting, immunoprecipitation, immunocytochemistry, immunofluorescence and immunohistochemistry, in particularly for the diagnosis in clinics using paraffin-embedded sections from patients suffering from various diseases and disorders where TRPV6 channels is involved. For example, antibodies generated against an epitope of the extracellular loop between S1 and S2 transmembrane domains or the extracellular part of the pore region were able to detect TRPV6 expression in paraffin-embedded sections from prostate cancer resection specimens. At the same time, the antibody did not give any signal in paraffin-embedded sections from healthy and benign hyperplasia prostate specimens.

Therefore, the invention relates to an antibody against human Transient Receptor Potential Vanilloid 6 (TRPV6) channel protein which binds to an extracellular epitope of hTRPV6 protein of SEQ ID NO: 1, in particular an epitope which is not glycosylated such as an epitope from the first extracellular region of human TRPV6 selected from any one of SEQ ID NO: 3 to 5, 7 and 8; preferably selected from any one of SEQ ID NO: 3, 7 and 8, or an epitope from the third extracellular region of human TRPV6 selected from SEQ ID NO: 14 or 16. The antibody is preferably an antibody which modulates the activity of human TRPV6 channel, preferably which activates human TRPV6 channel, and/or inhibits the proliferation of TRPV6-expressing cancer cells, preferably by inducing apoptosis of said cells.

In some embodiments, the antibody according to the invention or antigen-binding fragment thereof comprises heavy chain variable CDRs comprising at least one of, preferably all three of: a VH-CDR1 of SEQ ID NO: 27, a VH-CDR2 of SEQ ID NO: 28 and a VH-CDR3 of SEQ ID NO: 29, or a functional variant thereof, and light chain variable CDRs comprising at least one of, preferably all three of: a VL-CDR1 of SEQ ID NO: 17, a VL-CDR2 of amino acid sequence LVS and a VL-CDR3 of SEQ ID NO: 18, or a functional variant thereof.

In some embodiments, the antibody according to the invention or antigen-binding fragment thereof comprises:
a) heavy chain variable CDRs comprising at least one of, preferably all three of: a VH-CDR1 of SEQ ID NO: 49, a VH-CDR2 of SEQ ID NO: 50 and a VH-CDR3 of SEQ ID NO: 51, or a functional variant thereof; and light chain variable CDRs comprising at least one of, preferably all three of: a VL-CDR1 of SEQ ID NO: 38, a VL-CDR2 of amino acid sequence WAS and a VL-CDR3 of SEQ ID NO: 39, or a functional variant thereof; or
b) heavy chain variable CDRs comprising at least one of, preferably all three of: a VH-CDR1 of SEQ ID NO: 98, a VH-CDR2 of SEQ ID NO: 99 and a VH-CDR3 of SEQ ID NO: 100, or a functional variant thereof; and light chain variable CDRs comprising at least one of, preferably all three of: a VL-CDR1 of SEQ ID NO: 88, a VL-CDR2 of amino acid sequence SDS and a VL-CDR3 of SEQ ID NO: 89, or a functional variant thereof.

In some embodiments, the antibody according to the invention or antigen-binding fragment thereof comprises:
a) heavy chain variable CDRs comprising at least one of, preferably all three of: a VH-CDR1 of SEQ ID NO: 118, a VH-CDR2 of SEQ ID NO: 119 and a VH-CDR3 of SEQ ID NO: 120, or a functional variant thereof, and light chain variable CDRs comprising at least one of, preferably all three of: a VL-CDR1 of SEQ ID NO: 108, a VL-CDR2 of amino acid sequence YDS and a VL-CDR3 of SEQ ID NO: 109, or a functional variant thereof,
b) heavy chain variable CDRs comprising at least one of, preferably all three of: a VH-CDR1 of SEQ ID NO: 138, a VH-CDR2 of SEQ ID NO: 139 and a VH-CDR3 of SEQ ID NO: 140, or a functional variant thereof, and a light chain variable CDRs comprising at least one of, preferably all three of: a VL-CDR1 of SEQ ID NO: 128, a VL-CDR2 of amino acid sequence QDS and a VL-CDR3 of SEQ ID NO: 129 or a functional variant thereof;
c) heavy chain variable CDRs comprising at least one of, preferably all three of: a VH-CDR1 of SEQ ID NO: 158, a VH-CDR2 of SEQ ID NO: 159 and a VH-CDR3 of SEQ ID NO: 160, or a functional variant thereof, and light chain variable CDRs comprising at least one of, preferably all three of: a VL-CDR1 of SEQ ID NO: 148, a VL-CDR2 of amino acid sequence GDS and a VL-CDR3 of SEQ ID NO: 149, or a functional variant thereof, or
d) heavy chain variable CDRs comprising at least one of, preferably all three of: a VH-CDR1 of SEQ ID NO: 178, a VH-CDR2 of SEQ ID NO: 179 and a VH-CDR3 of SEQ ID NO: 180, or a functional variant thereof, and light chain variable CDRs comprising at least one of, preferably all three of: a VL-CDR1 of SEQ ID NO: 168, a VL-CDR2 of amino acid sequence YDS and a VL-CDR3 of SEQ ID NO: 169, or a functional variant thereof.

In some preferred embodiments, said monoclonal antibody is a humanized monoclonal antibody comprising a heavy chain variable domain comprising: a VH-FR1 of SEQ ID NO: 73, a VH-CDR1 of SEQ ID NO: 70, a VH-FR2 of SEQ ID NO: 74, a VH-CDR2 of SEQ ID NO: 71, a VH-FR3 of SEQ ID NO: 75, a VH-CDR3 of SEQ ID NO: 72 and a VH-FR4 of SEQ ID NO: 76, or a functional variant thereof, and a light chain variable domain comprising: a VL-FR1 of SEQ ID NO: 61, a VL-CDR1 of SEQ ID NO: 59, a VL-FR2 of SEQ ID NO: 62, a VL-CDR2 of amino acid sequence WAS, a VL-FR3 of SEQ ID NO: 63, a VL-CDR3 of SEQ ID NO: 60 and a VL-FR4 of SEQ ID NO: 64 or 65, or a functional variant thereof.

In some embodiments, the antibody according to the invention comprises s a heavy chain variable domain sequence and a light chain variable domain sequence having at least 90% identity with the pair of sequences: SEQ ID NO: 34 and SEQ ID NO: 23, respectively for the heavy chain variable domain sequence and light chain variable domain sequence. In some preferred embodiments, said antibody comprises a heavy chain sequence and a light chain sequence having at least 90% identity with the pair of sequences: SEQ ID NO: 35 and SEQ ID NO: 24; respectively for the heavy chain sequence and light chain sequence.

In some embodiments, the antibody according to the invention comprises a heavy chain variable domain sequence and a light chain variable domain sequence having at least 90% identity with any one of the following pair of sequences; SEQ ID NO: 56 and SEQ ID NO: 45 or 46; SEQ ID NO: 77, and SEQ ID NO: 66 or 67; SEQ ID NO: 105 and SEQ ID NO: 95 or 96; respectively for the heavy chain variable domain sequence and light chain variable domain sequence; preferably comprising a heavy chain variable domain sequence and a light chain variable domain sequence having at least 90% identity with any one of the following pair of sequences; SEQ ID NO: 56 and SEQ ID NO: 45 or 46; SEQ ID NO: 77, and SEQ ID NO: 66 or 67. In some preferred embodiments, said antibody comprises a heavy chain sequence and a light chain sequence having at least 90% identity with any one of the following pair of sequences: SEQ ID NO: 57 and SEQ ID NO: 47; SEQ ID NO: 78 or 80 and SEQ ID NO: 68; SEQ ID NO: 84 or 86 and SEQ ID NO: 82; SEQ ID NO: 106 or 107 and SEQ ID NO: 97, respectively for the heavy chain sequence and light chain sequence; preferably comprising a heavy chain variable domain sequence and a light chain variable domain sequence having at least 90% identity with a pair of sequences selected from: SEQ ID NO: 57 and SEQ ID NO: 47; SEQ ID NO: 78 or 80 and SEQ ID NO: 68; SEQ ID NO: 84 or 86 and SEQ ID NO: 82

In some embodiments, the antibody according to the invention comprises a heavy chain variable domain sequence and a light chain variable domain sequence having at least 90% identity with any one of the following pair of sequences; SEQ ID NO: 125 and SEQ ID NO: 115 or 116; SEQ ID NO: 145 and SEQ ID NO: 135 or 136; SEQ ID NO: 165 and SEQ ID NO: 155 or 156; SEQ ID NO: 185 and SEQ ID NO: 175 or 176; respectively for the heavy chain variable domain sequence and light chain variable domain sequence. In some preferred embodiments, said antibody comprises a heavy chain sequence and a light chain sequence having at least 90% identity with any one of the following pair of sequences: SEQ ID NO: 126 or 127 and SEQ ID NO: 117; SEQ ID NO: 146 or 147 and SEQ ID NO: 137; SEQ ID NO: 166 or 167 and SEQ ID NO: 157; SEQ ID NO: 186 or 187 and SEQ ID NO: 177 respectively for the heavy chain sequence and light chain sequence.

In some embodiments, the antibody according to the invention, is a polyclonal or monoclonal antibody, in particular recombinant, chimeric, and/or humanized monoclonal antibody, preferably of human IgG1 or IgG4 isotype.

In some embodiments, the antibody according to the invention, is coupled to a labeling agent or a therapeutic agent.

Another aspect of the invention relates to an extracellular peptide antigen from human TRPV6 protein which comprises a sequence having at least 90% identity with any one of SEQ ID NO: 3 to 5, 7, 8, 10, 14 or 16, and wherein the peptide antigen induces the production of an antibody according to the present disclosure.

Another aspect of the invention relates to an expression vector for the recombinant production of an antibody according to the present disclosure in a host cell, comprising at least one nucleic acid encoding the heavy and/or light chain of said antibody.

In some preferred embodiments, the expression vector comprises a pair of nucleic acid sequences having at least 90% identity with the pair of sequences: SEQ ID NO: 37 and 26.

In some other preferred embodiments, the expression vector comprises a pair of nucleic acid sequences having at least 90% identity with any one of the following pair of sequences SEQ ID NO: 48 and 58; SEQ ID NO: 69 and 79; SEQ ID NO: 69 and 81; SEQ ID NO: 83 and 85; SEQ ID NO: 83 and 87.

Another aspect of the invention relates to a pharmaceutical composition comprising at least an antibody according to the present disclosure, and a pharmaceutical acceptable vehicle.

Another aspect of the invention relates to the antibody according to the present disclosure for use as a medicament, in particular for use in the treatment of a disease associated with TRPV6 expression such as cancer, in particular selected from the group consisting of: endometrial cancers, leukemia, and carcinomas of the breast, pancreas, prostate, colon, ovarian, and thyroid; preferably said cancer is prostate cancer.

Another aspect of the invention relates to the use of the antibody according to the disclosure for the in vitro diagnosis or prognosis of a disease associated with TRPV6 expression such as cancer, in particular selected from the group consisting of: endometrial cancers, leukemia, and carcinomas of the breast, pancreas, prostate, colon, ovarian, and thyroid; preferably said cancer is prostate cancer.

### DETAILED DESCRIPTION OF THE INVENTION

### Antibody

The invention relates also to an antibody against human Transient Receptor Potential Vanilloid 6 (TRPV6) channel protein which binds to an extracellular epitope of said protein.

As used herein, the term “antibody” refers to “isolated antibody”. An antibody refers to a glycoprotein produced by lymphoid cells in response to stimulation with an immunogen. Antibodies possess the ability to react in vitro and in vivo specifically and selectively with an antigenic determinant or epitope eliciting their production or with an antigenic determinant closely related to the homologous antigen.

The expression “an antibody recognizing an antigen (X), “an antibody having specificity for an antigen (X)”, “an anti-X antibody”, “an antibody against X”, and an “antibody directed against” are used interchangeably herein with the term “an antibody which binds specifically to an antigen (X)”.

The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

In the present invention, the terms “antibody” and “immunoglobulin” are equivalent and used indifferently. Antibody is designated “Ab” and immunoglobulin is designated “Ig”.

As used herein, the term “recombinant antibody” refers to antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies. In some embodiments a recombinant human antibody of this invention has the same amino acid sequence as a naturally-occurring human antibody but differs structurally from the naturally occurring human antibody. For example, in some embodiments the glycosylation pattern is different as a result of the recombinant production of the recombinant human antibody. In some embodiments the recombinant human antibody is chemically modified by addition or subtraction of at least one covalent chemical bond relative to the structure of the human antibody that occurs naturally in humans.

An “epitope” or antigenic determinant refers to the portion of an antigen that is recognized by an antibody.

As used herein, the term “identity” refers to the sequence similarity between two polypeptide molecules or between two nucleic acid molecules. When a position in both compared sequences is occupied by the same base or same amino acid residue, then the respective molecules are identical at that position. The percentage of identity between two sequences corresponds to the number of matching positions shared by the two sequences divided by the number of positions compared and multiplied by 100. Generally, a comparison is made when two sequences are aligned to give maximum identity. The identity may be calculated by alignment using, for example, the GCG (Genetics Computer Group, Program Manual for the GCG Package, Version 7, Madison, Wisconsin) pileup program, or any of sequence comparison algorithms such as BLAST, FASTA or CLUSTALW. In the following description, the standard one letter amino acid code is used.

The antibody according to the invention binds to an epitope situated in one extracellular (EC) region of human TRPV6 protein. Human TRPV6 has the amino acid sequence UniProtKB/Swiss-Prot NP_061116.5 or Q9H1D0.3 (SEQ ID NO: 1). TRPV6 3D crystal structure has been determined (Saotome et al., Nature 2016, 534, 506-511).

TRPV6 has three extracellular (EC) regions: EC1 is situated between the first (S1) and the second (S2) transmembrane regions; EC2 is situated between the third (S3) and the fourth (S4) transmembrane regions; and the third extracellular regions is divided into two sub-regions: EC3a situated between the fifth (S5) transmembrane region and the intramembrane (IM) pore forming region and EC3b situated between the intramembrane (IM) pore forming region and the sixth transmembrane region (S6). Based on structure prediction, EC1 is predicted to correspond to positions 389 to 425 of SEQ ID NO: 1; EC2 is predicted to correspond to positions 484 to 489 of SEQ ID NO: 1; EC3a is predicted to correspond to positions 563 to 565 of SEQ ID NO: 1 and EC3b is predicted to correspond to positions 586 to 596 of SEQ ID NO: 1. However, the positions of the extracellular regions of hTRPV6 that are effectively accessible on the cell surface and may be bound by antibodies may vary slightly from the predicted positions. TRPV6 extracellular regions may be determined precisely using TRPV6 3D crystal structure (Saotome et al., Nature 2016, 534, 506-511).

The epitope bound by the antibody according to the invention may also comprise adjacent sequences (usually up to 5 amino acids; 1, 2, 3, 4 or 5 amino acids) from flanking transmembrane (TM) and/or intramembrane (IM) regions. The epitope bound by the antibody may be a variant of wild-type human TRPV6 sequence that does not modify the specificity of the antibody towards wild-type human TRPV6 protein. This means that the variant epitope induces cross-reactive antibodies which bind to both variant epitope and wild-type epitope with high affinity.

The extracellular epitope bound by the antibody of the invention is preferably from the first or third extracellular region of human TRPV6 (hTRPV6).

In some particular embodiments, the extracellular epitope bound by the antibody of the invention is not glycosylated. In particular, the epitope has no glycosylation site. The glysosylation site is preferably N-X-S or N-X-T, where X may be any amino acid.

In some embodiments, the antibody binds to an epitope from the first extracellular region (EC1) of human TRPV6 which is derived from the sequence SEQ ID NO: 2 (LLQEAYMTPKDDIRLVG); positions 412 to 428 of hTRPV6 or hTRPV6 412-428. In some preferred embodiments, the epitope is selected from the group consisting of SEQ ID NO: 3 to 5, 7, 8; preferably SEQ ID NO: 3, 7 or 8. SEQ ID NO: 3 (QEAYMTPKDDIRLVG) corresponds to hTRPV6 414-428; SEQ ID NO: 4 (QEAYMTPKDDIR) corresponds to hTRPV6 414-425; SEQ ID NO: 5 (LLQEAYMTPKDDIR) corresponds to hTRPV6 412-425; SEQ ID NO: 7 (EAYMTPKEEIRR) corresponds to hTRPV6 415-426 with D to E substitutions at the 8th and 9th position and L to R substitution at the last position of the peptide sequence; SEQ ID NO: 8 (EAYMTPKDDIRL) corresponds to hTRPV6 415-426.

In some embodiments, the antibody binds to an epitope from the third extracellular region (EC3) of human TRPV6. The epitope may be derived from EC3a, in particular from the sequence SEQ ID NO: 9 (IFQTEDPEELGHFYDYPMALFST; hTRPV6 551-573) or may be derived from EC3b. In some preferred embodiments, the epitope is selected from the group consisting of SEQ ID NO: 14 and 16; SEQ ID NO: 14 (TEDPEELGHFYDYPMA) corresponds to hTRPV6 554-569; SEQ ID NO: 16 (DGPANYNVDLPFMYS) corresponds to hTRPV6 582-596.

The antibody according to the invention recognizes specifically human TRPV6 protein, in particular human mature glycosylated TRPV6 protein on the plasma membrane. This means that the antibody has a relatively high affinity to its epitope of TRPV6, but do not substantially recognize and bind to peptides other than the one(s) of interest.

As used herein, the term “relatively high affinity” means a binding affinity between the antibody and the protein of interest of at least 10⁻⁶ M, and preferably of at least about 10⁻⁷ M and even more preferably 10⁻⁸ M to 10⁻¹⁰ M. Determination of such affinity is preferably conducted under standard competitive binding immunoassay conditions which is common knowledge to the person of ordinary skill in the art.

In some embodiments, the antibody modulates the activity of human TRPV6 channel. In a specific embodiment, the antibody activates human TRPV6 channel. In another specific embodiment the antibody inhibits human TRPV6 channel.

The modulation of TRPV6 channel activity by the antibody of the invention may be assayed according to standard techniques that are well-known in the art such as those disclosed in the examples, including with no limitations: whole-cell patch-clamp technique; Store-operated calcium entry (SOCE) assay as disclosed in Raphaël et al., 2014; radioactive uptake assay to measure ion transport across ion channels disclosed in Nimigean C M, Nat Protoc. 2006; 1(3):1207-12, and others.

In some embodiments, the antibody inhibits the proliferation of TRPV6-rexpressing cells, in particular cancer cells. The antibody advantageously induces apoptosis in TRPV6-expressing cells, such as cancer cells. The inhibition of proliferation or induction of apoptosis in TRPV6-expressing cells by the antibody of the invention may be assayed according to standard techniques that are well-known in the art such as those disclosed in the examples. For example, inhibition of proliferation may be measured using Cell survival assay using MTS or MTT; Cell cycle assay, Cell count assay, LDH leakage, total cellular protein measurement, neutral red, alamarBlue®, uridine incorporation assay, or by expression in western-blot/IHC of Ki-67, PCNA, CdK4, and cyclin D proteins. Induction of apoptosis may be measured using TUNEL assay, Hoechst staining, by detection of apoptosis markers such as phosphatidylserine exposure, caspase, calpain and cathepsin activation, changes in mitochondrial transmembrane potential, or cell membrane blebbing and nuclear condensation, DNA Ladder assay, cleaved capsase-3-assay, or Annexin V binding.

An antibody according to the invention may comprise a whole antibody or antigen-binding fragment thereof. The antibody fragment may be selected from the group consisting of: Fv, ScFv, Sc(Fv)2, DsFv, Fab, F(ab)₂, Fab′ fragments, diabodies and single domain antibodies (VHH). The variable regions of the antibody according to the invention may be associated with constant region domains such as IgA, IgM, IgE, IgG or IgD domains, in particular human constant region domains; preferably IgG, in particular human IgG1 or IgG4 constant domains. These constant regions may be further mutated or modified, by methods known in the art, in particular for modifying their binding capability towards Fc receptor or enhancing antibody half-life. The antibody may be glycosylated or non-glycosylated.

The antibody may be monoclonal or polyclonal, non-recombinant or recombinant, chimeric or humanized. A monoclonal antibody is a monospecific and bivalent immunoglobulin molecule. The term “antibody” is meant to encompass an aggregate, polymer, derivative, or conjugate of antibody or antibody fragment. Examples of derivative include variants and constructions using the antigen-binding fragment of such an antibody such as multivalent and/or multispecific antibodies.

In some embodiments, said antibody is a polyclonal antibody, for example a rabbit polyclonal antibody. A polyclonal antibody according to the invention is a monospecific antibody, which means that it is specific for an extracellular epitope of hTRPV6 protein. Such polyclonal antibody is generally obtained by immunization with a peptide having the sequence of the extracellular epitope or a closely related sequence that induces cross-reactive antibodies as defined above. In some preferred embodiments, the polyclonal antibody binds to the epitope of SEQ ID NO: 3.

In some embodiments, said antibody is a monoclonal antibody (mAb), preferably human, humanized or chimeric. A chimeric antibody has human constant domains and variable domains from a non-human source, generally mouse (human/mouse chimeric antibody). The monoclonal antibody is preferably a recombinant antibody.

In some particular embodiments, the monoclonal antibody or antigen-binding fragment thereof which binds to the epitope of SEQ ID NO: 8 and the variant epitope thereof of SEQ ID NO: 7 comprises heavy chain variable CDRs comprising at least one of, preferably all three of: a VH-CDR1 of SEQ ID NO: 27, a VH-CDR2 of SEQ ID NO: 28 and a VH-CDR3 of SEQ ID NO: 29, or a functional variant thereof, and light chain variable CDRs comprising at least one of, preferably all three of: a VL-CDR1 of SEQ ID NO: 17, a VL-CDR2 of amino acid sequence LVS and a VL-CDR3 of SEQ ID NO: 18, or a functional variant thereof. Preferably, the monoclonal antibody or antigen-binding fragment thereof which binds to the epitope of SEQ ID NO: 8 and the variant epitope thereof of SEQ ID NO: 7 comprises a heavy chain variable domain and a light chain variable domain selected from: a heavy chain variable domain comprising at least one of, preferably all three of: a VH-CDR1 of SEQ ID NO: 27, a VH-CDR2 of SEQ ID NO: 28 and a VH-CDR3 of SEQ ID NO: 29, or a functional variant thereof, and a light chain variable domain comprising at least one of, preferably all three of: a VL-CDR1 of SEQ ID NO: 17, a VL-CDR2 of amino acid sequence LVS and a VL-CDR3 of SEQ ID NO: 18, or a functional variant thereof.

In some particular embodiments, the monoclonal antibody or antigen-binding fragment thereof binds which binds to the epitope of SEQ ID NO: 14 comprises:
a) heavy chain variable CDRs comprising at least one of, preferably all three of: a VH-CDR1 of SEQ ID NO: 49, a VH-CDR2 of SEQ ID NO: 50 and a VH-CDR3 of SEQ ID NO: 51, or a functional variant thereof, and light chain variable CDRs comprising at least one of, preferably all three of: a VL-CDR1 of SEQ ID NO: 38, a VL-CDR2 of amino acid sequence WAS and a VL-CDR3 of SEQ ID NO: 39, or a functional variant thereof, or
b) heavy chain variable CDRs comprising at least one of, preferably all three of: a VH-CDR1 of SEQ ID NO: 98, a VH-CDR2 of SEQ ID NO: 99 and a VH-CDR3 of SEQ ID NO: 100, or a functional variant thereof, and light chain variable CDRs comprising at least one of, preferably all three of: a VL-CDR1 of SEQ ID NO: 88, a VL-CDR2 of amino acid sequence SDS and a VL-CDR3 of SEQ ID NO: 89, or a functional variant thereof.

In some preferred embodiments, the monoclonal antibody or antigen-binding fragment thereof binds which binds to the epitope of SEQ ID NO: 14 comprises a heavy chain variable domain comprising at least one of, preferably all three of: a VH-CDR1 of SEQ ID NO: 49, a VH-CDR2 of SEQ ID NO: 50 and a VH-CDR3 of SEQ ID NO: 51, or a functional variant thereof; and a light chain variable domain comprising at least one of, preferably all three of: a VL-CDR1 of SEQ ID NO: 38, a VL-CDR2 of amino acid sequence WAS and a VL-CDR3 of SEQ ID NO: 39, or a functional variant thereof.

In some other embodiments, the monoclonal antibody or antigen-binding fragment thereof binds which binds to the epitope of SEQ ID NO: 14 comprises a heavy chain variable domain comprising at least one of, preferably all three of: a VH-CDR1 of SEQ ID NO: 98, a VH-CDR2 of SEQ ID NO: 99 and a VH-CDR3 of SEQ ID NO: 100, or a functional variant thereof; and a light chain variable domain comprising at least one of, preferably all three of: a VL-CDR1 of SEQ ID NO: 88, a VL-CDR2 of amino acid sequence SDS and a VL-CDR3 of SEQ ID NO: 89, or a functional variant thereof.

In some particular embodiments, the monoclonal antibody or antigen-binding fragment thereof binds which binds to the epitope of SEQ ID NO: 16 comprises:
a) heavy chain variable CDRs comprising at least one of, preferably all three of: a VH-CDR1 of SEQ ID NO: 118, a VH-CDR2 of SEQ ID NO: 119 and a VH-CDR3 of SEQ ID NO: 120, or a functional variant thereof, and light chain variable CDRs comprising at least one of, preferably all three of: a VL-CDR1 of SEQ ID NO: 108, a VL-CDR2 of amino acid sequence YDS and a VL-CDR3 of SEQ ID NO: 109, or a functional variant thereof;
b) heavy chain variable CDRs comprising at least one of, preferably all three of: a VH-CDR1 of SEQ ID NO: 138, a VH-CDR2 of SEQ ID NO: 139 and a VH-CDR3 of SEQ ID NO: 140, or a functional variant thereof, and a light chain variable CDRs comprising at least one of, preferably all three of: a VL-CDR1 of SEQ ID NO: 128, a VL-CDR2 of amino acid sequence QDS and a VL-CDR3 of SEQ ID NO: 129 or a functional variant thereof;
c) heavy chain variable CDRs comprising at least one of, preferably all three of: a VH-CDR1 of SEQ ID NO: 158, a VH-CDR2 of SEQ ID NO: 159 and a VH-CDR3 of SEQ ID NO: 160, or a functional variant thereof; and light chain variable CDRs comprising at least one of, preferably all three of: a VL-CDR1 of SEQ ID NO: 148, a VL-CDR2 of amino acid sequence GDS and a VL-CDR3 of SEQ ID NO: 149, or a functional variant thereof; or
d) heavy chain variable CDRs comprising at least one of, preferably all three of: a VH-CDR1 of SEQ ID NO: 178, a VH-CDR2 of SEQ ID NO: 179 and a VH-CDR3 of SEQ ID NO: 180, or a functional variant thereof; and light chain variable CDRs comprising at least one of, preferably all three of: a VL-CDR1 of SEQ ID NO: 168, a VL-CDR2 of amino acid sequence YDS and a VL-CDR3 of SEQ ID NO: 169, or a functional variant thereof.

In some particular embodiments, the monoclonal antibody or antigen-binding fragment thereof binds which binds to the epitope of SEQ ID NO: 16 comprises a heavy chain variable domain and a light chain variable domain selected from:
a) a heavy chain variable domain comprising at least one of, preferably all three of: a VH-CDR1 of SEQ ID NO: 118, a VH-CDR2 of SEQ ID NO: 119 and a VH-CDR3 of SEQ ID NO: 120, or a functional variant thereof, and a light chain variable domain comprising at least one of, preferably all three of: a VL-CDR1 of SEQ ID NO: 108, a VL-CDR2 of amino acid sequence YDS and a VL-CDR3 of SEQ ID NO: 109, or a functional variant thereof;
b) a heavy chain variable domain comprising at least one of, preferably all three of: a VH-CDR1 of SEQ ID NO: 138, a VH-CDR2 of SEQ ID NO: 139 and a VH-CDR3 of SEQ ID NO: 140, or a functional variant thereof, and a light chain variable domain comprising at least one of, preferably all three of: a VL-CDR1 of SEQ ID NO: 128, a VL-CDR2 of amino acid sequence QDS and a VL-CDR3 of SEQ ID NO: 129 or a functional variant thereof;
c) a heavy chain variable domain comprising at least one of, preferably all three of: a VH-CDR1 of SEQ ID NO: 158, a VH-CDR2 of SEQ ID NO: 159 and a VH-CDR3 of SEQ ID NO: 160, or a functional variant thereof, and a light chain variable domain comprising at least one of, preferably all three of: a VL-CDR1 of SEQ ID NO: 148, a VL-CDR2 of amino acid sequence GDS and a VL-CDR3 of SEQ ID NO: 149, or a functional variant thereof; and
d) a heavy chain variable domain comprising at least one of, preferably all three of: a VH-CDR1 of SEQ ID NO: 178, a VH-CDR2 of SEQ ID NO: 179 and a VH-CDR3 of SEQ ID NO: 180, or a functional variant thereof, and a light chain variable domain comprising at least one of, preferably all three of: a VL-CDR1 of SEQ ID NO: 168, a VL-CDR2 of amino acid sequence YDS and a VL-CDR3 of SEQ ID NO: 169, or a functional variant thereof.

As used herein, “functional variant” with respect to a variant of an antibody sequence (CDR, FR or others), means that the antibody comprising the sequence variant recognizes specifically human TRPV6 protein. It is contemplated that monoclonal antibodies or antigen-binding fragment thereof may have 1, 2, 3, 4, 5, 6, or more alterations in the amino acid sequence of 1, 2, 3, 4, 5, or 6 CDRs of monoclonal antibodies provided herein. It is contemplated that the amino acid in position 1, 2, 3, 4, 5, 6, 7, 8, 9, 10, 11, 12 or 13 of CDR1, CDR2, CDR3, CDR4, CDR5, or CDR6 of the VJ or VDJ region of the light or heavy variable region of antibodies may have an insertion, deletion, or substitution with a conserved or non-conserved amino acid. Such amino acids that can either be substituted or constitute the substitution are disclosed below. In some particular embodiments, the monoclonal antibodies or antigen-binding fragment have 1 or 2 conservative substitutions in the amino acid sequence of 1, 2, 3, 4, 5, or 6 CDRs of monoclonal antibodies provided herein. It is also contemplated that monoclonal antibodies or antigen-binding fragment thereof may have 1, 2, 3, 4, 5, 6, 7, 8, 9, 10 or more alterations in the amino acid sequence of 1, 2, 3, 4, 5, 6, 7, 8 FRs of monoclonal antibodies provided herein. It is contemplated that the FR sequences have an insertion, deletion, or substitution with a conserved or non-conserved amino acid. Such amino acids that can either be substituted or constitute the substitution are disclosed above. In some particular embodiments, the monoclonal antibodies or antigen-binding fragment have 1, 2, 3, 4, 5; preferably 1 or 2 conservative substitutions in the amino acid sequence of 1, 2, 3, 4, 5, 6, 7, 8 FRs of monoclonal antibodies provided herein.

In some embodiments, the substitutions are conservative substitutions, i.e., substitutions of one amino acid with another having similar chemical or physical properties (size, charge or polarity), which substitution generally does not adversely affect the biochemical, biophysical and/or biological properties of the antibody. In particular, the substitution does not disrupt the interaction of the antibody with human TRPV6 protein. Said conservative substitution(s) are advantageously chosen within one of the following five groups: Group 1-small aliphatic, non-polar or slightly polar residues (A, S, T, P, G); Group 2-polar, negatively charged residues and their amides (D, N, E, Q); Group 3-polar, positively charged residues (H, R, K); Group 4-large aliphatic, nonpolar residues (M, L, I, V, C); and Group 5-large, aromatic residues (F, Y, W).

In some embodiments, said antibody is a monoclonal antibody which binds to the epitope bound by the antibody having the six VH-CDR and the six VL-CDR sequences as defined above.

In some preferred embodiments, the monoclonal antibody or antigen-binding fragment thereof comprises heavy chain variable CDRs comprising at least one of, preferably all three of: a VH-CDR1 of SEQ ID NO: 49, a VH-CDR2 of SEQ ID NO: 50 and a VH-CDR3 of SEQ ID NO: 51, or a functional variant thereof, and light chain variable CDRs comprising at least one of, preferably all three of: a VL-CDR1 of SEQ ID NO: 38, a VL-CDR2 of amino acid sequence WAS and a VL-CDR3 of SEQ ID NO: 39, or a functional variant thereof. Preferably the monoclonal antibody or antigen-binding fragment thereof comprises a heavy chain variable domain comprising at least one of, preferably all three of: a VH-CDR1 of SEQ ID NO: 49, a VH-CDR2 of SEQ ID NO: 50 and a VH-CDR3 of SEQ ID NO: 51, or a functional variant thereof; and a light chain variable domain comprising at least one of, preferably all three of: a VL-CDR1 of SEQ ID NO: 38, a VL-CDR2 of amino acid sequence WAS and a VL-CDR3 of SEQ ID NO: 39, or a functional variant thereof.

In some more preferred embodiments, said antibody is a humanized monoclonal antibody or antigen-binding fragment thereof which binds to the epitope of SEQ ID NO: 14 and comprises a heavy chain variable domain comprising: a VH-FR1 of SEQ ID NO: 73, a VH-CDR1 of SEQ ID NO: 70, a VH-FR2 of SEQ ID NO: 74, a VH-CDR2 of SEQ ID NO: 71, a VH-FR3 of SEQ ID NO: 75, a VH-CDR3 of SEQ ID NO: 72 and a VH-FR4 of SEQ ID NO: 76, or a functional variant thereof, and a light chain variable domain comprising: a VL-FR1 of SEQ ID NO: 61, a VL-CDR1 of SEQ ID NO: 59, a VL-FR2 of SEQ ID NO: 62, a VL-CDR2 of amino acid sequence WAS, a VL-FR3 of SEQ ID NO: 63, a VL-CDR3 of SEQ ID NO: 60 and a VL-FR4 of SEQ ID NO: 64 or 65, or a functional variant thereof.

In some particular embodiments, the antibody or antigen-binding fragment thereof according to the present disclosure comprises a heavy chain variable domain comprising an amino acid sequence having at least 90% identity with SEQ ID NO: 34, and a light chain variable domain comprising an amino acid sequence having at least 90% identity with SEQ ID NO: 23.

In some particular embodiments, the antibody or antigen-binding fragment thereof according to the present disclosure comprises a heavy chain variable domain and a light chain variable domain selected from:
a) a heavy chain variable domain comprising an amino acid sequence having at least 90% identity with SEQ ID NO: 56, and a light chain variable domain comprising an amino acid sequence having at least 90% identity with SEQ ID NO: 45 or 46;
b) a heavy chain variable domain comprising an amino acid sequence having at least 90% identity with SEQ ID NO: 77, and a light chain variable domain comprising an amino acid sequence having at least 90% identity with SEQ ID NO: 66 or 67;
c) a heavy chain variable domain comprising an amino acid sequence having at least 90% identity with SEQ ID NO: 105, and a light chain variable domain comprising an amino acid sequence having at least 90% identity with SEQ ID NO: 95 or 96;
d) a heavy chain variable domain comprising an amino acid sequence having at least 90% identity with SEQ ID NO: 125, and a light chain variable domain comprising an amino acid sequence having at least 90% identity with SEQ ID NO: 115 or 116;
e) a heavy chain variable domain comprising an amino acid sequence having at least 90% identity with SEQ ID NO: 145, and a light chain variable domain comprising an amino acid sequence having at least 90% identity with SEQ ID NO: 135 or 136;
f) a heavy chain variable domain comprising an amino acid sequence having at least 90% identity with SEQ ID NO: 165, and a light chain variable domain comprising an amino acid sequence having at least 90% identity with SEQ ID NO: 155 or 156; and g) a heavy chain variable domain comprising an amino acid sequence having at least 90% identity with SEQ ID NO: 185, and a light chain variable domain comprising an amino acid sequence having at least 90% identity with SEQ ID NO: 175 or 176.

In some preferred embodiments, the antibody or antigen-binding fragment thereof according to the present disclosure comprises: a heavy chain variable domain comprising an amino acid sequence having at least 90% identity with SEQ ID NO: 56, and a light chain variable domain comprising an amino acid sequence having at least 90% identity with SEQ ID NO: 45 or 46; or a heavy chain variable domain comprising an amino acid sequence having at least 90% identity with SEQ ID NO: 77, and a light chain variable domain comprising an amino acid sequence having at least 90% identity with SEQ ID NO: 66 or 67.

In some more particular embodiments, said antibody or antigen-binding fragment thereof comprises: a heavy chain amino acid sequence having at least 90% identity with SEQ ID NO: 35 and a light chain amino acid sequence having at least 90% identity with SEQ ID NO: 24.

In some more particular embodiments, said antibody or antigen-binding fragment thereof comprises:
a) a heavy chain amino acid sequence having at least 90% identity with SEQ ID NO: 57 and a light chain amino acid sequence having at least 90% identity with SEQ ID NO: 47;
b) a heavy chain amino acid sequence having at least 90% identity with SEQ ID NO: 78 or 80 and a light chain amino acid sequence having at least 90% identity with SEQ ID NO: 68;
c) a heavy chain amino acid sequence having at least 90% identity with SEQ ID NO: 84 or 86 and a light chain amino acid sequence having at least 90% identity with SEQ ID NO: 82;
d) a heavy chain amino acid sequence having at least 90% identity with SEQ ID NO: 106 or 107 and a light chain amino acid sequence having at least 90% identity with SEQ ID NO: 97;
e) a heavy chain amino acid sequence having at least 90% identity with SEQ ID NO: 126 or 127 and a light chain amino acid sequence having at least 90% identity with SEQ ID NO: 117;
f) a heavy chain amino acid sequence having at least 90% identity with SEQ ID NO: 146 or 147 and a light chain amino acid sequence having at least 90% identity with SEQ ID NO: 137;
g) a heavy chain amino acid sequence having at least 90% identity with SEQ ID NO: 166 or 167 and a light chain amino acid sequence having at least 90% identity with SEQ ID NO: 157; or
h) a heavy chain amino acid sequence having at least 90% identity with SEQ ID NO: 186 or 187 and a light chain amino acid sequence having at least 90% identity with SEQ ID NO: 177.

In some preferred embodiments, the antibody or antigen-binding fragment thereof according to the present disclosure comprises a heavy chain amino acid sequence and a light chain amino acid sequence having at least 90% identity with any one of the following pairs of sequences: SEQ ID NO: 47 and 57; SEQ ID NO: 68 and 78, SEQ ID NO: 68 and 80, SEQ ID NO: 82 and 84, and SEQ ID NO: 82 and 86.

In some embodiments, the antibody of the invention comprise sequences as presented in Table I.

The antibody of the invention can be produced by the conventional techniques known to those skilled in the art. For example, monoclonal antibodies are produced from hybridomas obtained by fusion of B lymphocytes of an animal immunized with CEA antigen, with myelomas, according to the technique of Kohler and Milstein (Nature, 1975, 256, 495-497); the hybridomas are cultured in vitro, in particular in fermenters. Chimeric and/or humanized recombinant antibody and antibody fragments can be prepared from hybridoma cells specific for the antigen by the conventional techniques of recombinant DNA cloning and expression. Human antibody can be obtained from a transgenic mouse possessing human immunoglobulin loci.

In some embodiments, the antibody is modified. In particular, the antibody constant regions may be mutated or modified, by methods known in the art, in particular for modifying their binding capability towards Fc receptor, enhancing antibody half-life or coupling to an agent of interest such as. a labeling agent or a therapeutic agent. For example, covalent coupling of the agent to the antibody may be achieved by incorporating a reactive group in the antibody, and then using the group to link the agent covalently. Alternatively, covalent coupling may be achieved by engineering a fusion protein.

In some embodiments, the antibody is coupled to a labeling agent. The labeling agent is any agent which produces a detectable and/or quantifiable signal, in particular a radioactive, magnetic or luminescent (radioluminescent, chemiluminescent, bioluminescent, fluorescent or phosphorescent) agent. The antibody may be labeled directly or indirectly, via covalent or non-covalent bonds, using standard conjugation techniques that are well-known to those skilled in the art. Directly detectable labels include radioisotopes and fluorophores. Indirectly detectable labels are detected by labeling with additional reagents that enable the detection.

Indirectly detectable labels include, for example, chemiluminescent agents, enzymes that produce visible or colored reaction products, and a ligand-detectable ligand binding partner, where a ligand (hapten, antibody, antigen, biotin) may be detected by binding to a labelled ligand-specific binding partner. The detectable label according to the invention can be of any type; it can in particular be a fluorophore, for example fluorescein or luciferase; a radioisotope, in particular suitable for scintigraphy, for example ^{99m}Tc; or an enzyme, for example horseradish peroxidase.

In some other embodiments, the antibody is coupled to a drug, for example an anticancer drug.

The antibody according to the invention is used to target cells expressing TRPV6, in particular tumor cells, for diagnostic and therapeutic purposes. The target cells are preferably overexpressing TRPV6.

In the present invention “cells overexpressing TRPV6”, in particular “tumor or cancer cells overexpressing TRPV6” refer to cells such as tumor or cancer cells exhibiting a level of expression of TRPV6 which is significantly higher compared to that of normal cells of the corresponding tissue or organ in a healthy individual. TRPV6 expression level is measured by standard gene expression assays based on quantitative analysis of mRNA (RT-PCR and others) or protein (immunoassay such as ELISA and others).

The invention encompasses the use of mixtures or combinations of antibodies such as mixtures of different anti-TRPV6 antibodies according to the invention or mixtures of antibodies according to the invention and other antibodies.

“a”, “an”, and “the” include plural referents, unless the context clearly indicates otherwise. As such, the term “a” (or “an”), “one or more” or “at least one” can be used interchangeably herein.

### Peptide Antigen and Use for Antibody Production

The invention relates to an extracellular peptide antigen from human TRPV6 protein which induces the production of an antibody according to the invention.

The peptide of the invention is an isolated, recombinant or synthetic peptide, derived from human Transient Receptor Potential Vanilloid 6 (TRPV6) protein which is different from TRPV6 protein.

The peptide of the invention is an extracellular peptide derived from one of the extracellular (EC) regions of human TRPV6 protein as defined above. The peptide according to the invention may also comprise adjacent sequences (usually up to 5 amino acids, 1, 2; 3, 4 or 5 amino acids) from flanking transmembrane (TM) and/or intramembrane (IM) regions. The peptide of the invention is preferably derived from the first or third extracellular regions of human TRPV6.

The peptide of the invention is an antigenic peptide which means that immunization of a non-human mammal, for example mouse or rabbit, with the peptide according to the invention, induces the production of an antibody according to the invention.

In some embodiments, the peptide is derived from the first extracellular region (EC1) of human TRPV6, in particular from the sequence SEQ ID NO: 2 (LLQQKLLQEAYMTPKDDIRLVG); positions 412 to 428 of hTRPV6 or hTRPV6 412-428-). In some preferred embodiments, the peptide comprises or consists of a sequence selected from the group consisting of the sequences SEQ ID NO: 3, 4, 5, 7 or 8 and the sequences having at least 70% amino acid identity with any one of said sequences; preferably the sequence SEQ ID NO: 3, 7 or 8. SEQ ID NO: 3 (QEAYMTPKDDIRLVG) corresponds to hTRPV6 414-428; SEQ ID NO: 4 (QEAYMTPKDDIR) corresponds to hTRPV6 414-425; SEQ ID NO: 5 (LLQEAYMTPKDDIR) corresponds to hTRPV6 412-425; SEQ ID NO: 7 (EAYMTPKEEIRR) corresponds to hTRPV6 415-426 with D to E substitutions at the 8th and 9th position and L to R substitution at the last position of the peptide sequence; SEQ ID NO: 8 (EAYMTPKDDIRL) corresponds to hTRPV6 415-426.

In some embodiments, the peptide is derived from the third extracellular region (EC3) of human TRPV6. The peptide may be derived from EC3a, in particular from the sequence SEQ ID NO: 9 (IFQTEDPEELGHFYDYPMALFST; hTRPV6 551-573) or may be derived from EC3b. In some preferred embodiments, the peptide comprises or consists of a sequence selected from the group consisting of the sequences SEQ ID NO: 14 and 16 and the sequences having at least 70% amino acid identity with any one of said sequences. SEQ ID NO: 14 (TEDPEELGHFYDYPMA) corresponds to hTRPV6 554-569. SEQ ID NO: 16 (DGPANYNVDLPFMYS) corresponds to hTRPV6 582-596. Preferably, said peptide has at least 75%, 80%, 85%, 87%, 90%, 95%, 98% or 99% identity with any one of said sequences. More preferably, said peptide has at least 95%, 98% or 99% identity with any one of said sequences.

The peptide usually consists of a sequence of up to 50 amino acids, preferably 20, 25, 30, 35, 40, 45 amino acids derived from human TRPV6.

The invention encompasses a peptide comprising or consisting of a chain of natural amino acids (20 gene-encoded amino acids (A, R, N, D, C, Q, E, G, H, I, L, K, M, F, P, S, T, W, X and Y) in a L- and/or D-configuration) linked via a peptide bond and furthermore comprises peptidomimetics of such peptide where the amino acid(s) and/or peptide bond(s) have been replaced by functional analogues. Such functional analogues include all known amino acids other than said 20 gene-encoded amino acids.

The invention also encompasses modified peptides derived from the above peptides by introduction of any chemical modification into one or more amino acid residues, peptide bonds, N- and/or C-terminal ends of the peptide, as long as the modified peptide is functional. These modifications which are introduced into the peptide by the conventional methods known to those skilled in the art, include, in a non-limiting manner: the substitution of a natural amino acid with a non-proteinogenic amino acid (D amino acid or amino acid analog); the modification of the peptide bond, in particular with a bond of the retro or retro-inverso type or a bond different from the peptide bond; the cyclization, and the addition of a chemical group to the side chain or the end(s) of the peptide, in particular for coupling an agent of interest to the peptide of the invention. These modifications may be used to increase its antigenicity, immunogenicity and/or bioavailability, or to label the peptide.

In some embodiments, the peptide is coupled to a carrier protein to increase the immunogenicity of the peptide. The peptide of the invention may be coupled to any carrier protein used to prepare antibodies. In some particular embodiments, the peptide is coupled to KLH protein, for example via the peptide N-ter. In some preferred embodiments, the peptide is any one of SEQ ID NO: 3, 4, 5, 7 or 8 coupled to a carrier protein such as KLH via the peptide N-ter.

In some embodiments, the peptide comprises an additional amino acid residue, in particular a lysine at the N-ter or C-ter. For example, the peptide is SEQ ID NO: 6 or SEQ ID NO: 15. In some other embodiments, the peptide comprises a spacer sequence at the N-ter or C-ter. Such modifications are useful for coupling an agent of interest to the peptide of the invention.

The peptide according to the invention is prepared by the conventional techniques known to those skilled in the art, in particular by solid-phase or liquid-phase synthesis or by expression of a recombinant DNA in a suitable cell system (eukaryotic or prokaryotic). The peptide is usually solid-phase synthesized, according to the Fmoc technique, originally described by Merrifield et al. (J. Am. Chem. Soc., 1964, 85: 2149-) and purified by reverse-phase high performance liquid chromatography.

The peptide according to the invention is used for the production of an antibody according to the invention.

Therefore, the invention relates to the use of the peptide according to the invention for the production of an antibody according to the invention.

The invention relates also to a method of producing an anti-TRPV6 antibody according to the invention, comprising the steps of:
a) immunizing a non-human mammal, for example laboratory rodent such as rabbit or mouse, with the peptide according to the invention, to induce the production of anti-TRPV6 antibodies by the B cells of said mammal;
b) collecting said anti-TRPV6 antibodies or B cells from the mammal.

The immunization step is performed according to standard protocols which are known in the art, such as by using a peptide coupled to a carrier such as KLH protein.

The antibodies may be harvested from the serum of the immunized mammal. The B cells may be isolated from the spleen of the immunized mammal.

The method may further comprise B-cell immortalization, for example by fusion of the B-cells with a myeloma cell line, a lymphoblastoid cell line, lymphoma cells or an heteromyeloma cell line, according to standard hybridoma production techniques. Preferably, the B-cells are immortalized by fusion with a murine myeloma cell line, more preferably a murine myeloma cell line like the SP2/0 cell line, which does not produce any murine antibody, is immortalized, and possesses the entire secretion machinery necessary for the secretion of immunoglobulins. The immortalized B-cells are screened for specific antibody production using conventional assays like ELISA. After screening, they are usually cloned using standard methods. The antibodies which are secreted by the immortalized B-cells are harvested from the extracellular medium and usually further purified by conventional techniques known to the persons skilled in the art, such as affinity chromatography. Alternatively, VH and VL fragments of the anti-TRPV6 antibodies may be cloned from the B cells producing the anti-TRPV6 antibody according to the invention and recombinant antibodies may be produced according to standard techniques which are well-known in the art.

The anti-TRPV6 antibody according to the invention may also be produced by screening of a phage display library. In particular, VH and VL fragments of the anti-TRPV6 antibodies may be also screened from a phage display library using a peptide antigen according to the invention and recombinant antibodies may be produced according to standard techniques which are well-known in the art.

The invention also encompasses the antibody obtained or susceptible to be obtained by the method of producing an anti-TRPV6 antibody according to the invention

### Polynucleotide and Vector

The invention relates also to an isolated polynucleotide encoding the antibody of the invention in expressible form.

The polynucleotide encoding the antibody in expressible form refers to a nucleic acid molecule which, upon expression in a cell or a cell-free system, results in a functional peptide or antibody.

The polynucleotide, either synthetic or recombinant, may be DNA, RNA or combination thereof, either single- and/or double-stranded. The polynucleotide is operably linked to at least one transcriptional regulatory sequence and, optionally to at least one translational regulatory sequence. Preferably the polynucleotide comprises a coding sequence which is optimized for the host in which the peptide or antibody is expressed.

In some embodiments, said polynucleotide encodes at least the VH and/or VL domain of a monoclonal antibody according to the invention. In some preferred embodiments, the polynucleotide encoding the VH domain comprises a sequence having at least 80% identity with SEQ ID NO: 36 and the polynucleotide encoding the VL domain comprises a sequence having at least 80% identity with SEQ ID NO: 25. In some particular embodiments, the polynucleotide encodes the heavy and/or light chains of the antibody according to the present disclosure. In some preferred embodiments, the heavy and light chains of the antibody according to the present disclosure are encoded by at least one polynucleotide comprising a pair of sequences selected from the group consisting of: SEQ ID NO: 37 and 26 and the sequences having at least 80% identity with the preceding sequences.

In some other preferred embodiments, the heavy and light chains of the antibody according to the present disclosure are encoded by at least one polynucleotide comprising a pair of sequences selected from the group consisting of: SEQ ID NO: 48 and 58; SEQ ID NO: 69 and 79; SEQ ID NO: 69 and 81; SEQ ID NO: 83 and 85; SEQ ID NO: 83 and 87, and the sequences having at least 80% identity with the preceding sequences. The polynucleotide(s) may comprise or consist of a sequence having at least 85%, 90%, 95%, 96%, 97%, 98%, 99%, or 100% identity with the above disclosed sequences.

The polynucleotide according to the invention is prepared by the conventional methods known in the art. For example, it is produced by amplification of a nucleic sequence by PCR or RT-PCR, by screening genomic DNA libraries by hybridization with a homologous probe, or else by total or partial chemical synthesis.

Another aspect of the invention is a recombinant vector comprising said polynucleotide; preferably comprising a pair of polynucleotide sequences encoding at least the VH and/or VL domain of a monoclonal antibody according to the invention as defined above. The recombinant vector is advantageously an expression vector capable of expressing said polynucleotide when delivered into a host cell such as prokaryotic or eukaryotic cell, for example mammalian or bacterial cell. Recombinant vectors include usual vectors used in genetic engineering, vaccines and gene therapy including for example plasmids and viral vectors.

The recombinant vectors are constructed and introduced into host cells by the conventional recombinant DNA and genetic engineering techniques, which are known in the art.

Thus, a further aspect of the invention provides a host cell transformed with said polynucleotide or recombinant vector.

The polynucleotide, vector, cell of the invention are useful for the production of the antibodies of the invention using well-known recombinant DNA techniques.

### Pharmaceutical Composition and Therapeutic Use

The present invention also relates to a pharmaceutical composition comprising, as active substance, at least one antibody, polynucleotide and/or vector, according to the invention, in association with at least one pharmaceutically acceptable vehicle.

The pharmaceutical composition is formulated for administration by a number of routes, including but not limited to oral, parenteral and local. The pharmaceutical vehicles are those appropriate to the planned route of administration, which are well known in the art.

The pharmaceutical composition comprises a therapeutically effective amount of the antibody/polynucleotide/vector/sufficient to show a positive medical response in the indidual to whom it is administered. A positive medical response refers to the reduction of subsequent (preventive treatment) or established (therapeutic treatment) disease symptoms. The positive medical response comprises a partial or total inhibition of the symptoms of the disease. A positive medical response can be determined by measuring various objective parameters or criteria such as objective clinical signs of the disease and/or the increase of survival. A medical response to the composition according to the invention can be readily verified in appropriate animal models of the disease which are well-known in the art and illustrated in the examples of the present application.

The pharmaceutically effective dose depends upon the composition used, the route of administration, the type of mammal (human or animal) being treated, the physical characteristics of the specific mammal under consideration, concurrent medication, and other factors, that those skilled in the medical arts will recognize.

In some embodiments, the pharmaceutical composition comprises another active agent wherein said active agent is a pharmaceutical agent or therapeutic capable of preventing, treating or ameliorating a disease in humans or animals. The active agent may be a protein including an antibody, an oligonucleotide including an antisense oligonucleotide, peptide nucleic acid (PNA), small interfering RNA, locked nucleic acids (LNA), phosphorodiamidate morpholino oligonucleotides (PMO) and decoy DNA molecule, a plasmid, an aptamer including DNA, RNA or peptide aptamer, a small or large chemical drug, or mixtures thereof. In particular, the active agent may be an anticancer and/or immunomodulatory agent. The anticancer agent may be a chemotherapeutic agent. The anticancer agent may also be another antibody such as but not limited to Alacizumab, Amivantamab, Atezolizumab, BCD-100, Bemarituzumab, Bevacizumab, Cabiralizumab, Catumaxomab, Cetrelimab, Cetuximab, Ertumaxomab, Ficlatuzumab, Futuximab, Margetuximab, Necitumumab, Oportuzumab, Pankomab, Tomuzotuximab and others. The immunomodulatory agent may be an anti-PD1 or anti-PDL1 agent, in particular an anti-PD1 or anti-PDL1 antibody; a cytokine, mushroom glycanes, plant-derived immunomodulators and anti-cancer agents, statin, metformin, and angiotensin receptor blockers (ARBs), anthracyclines, thalidomides, lenalidomides, and hypomethylating drugs, or others. The anticancer or and/or immunomodulatory agent may be advantageously linked to the antibody according to the invention by standard means that are known in the art such as by covalent coupling or making of a genetic fusion.

The invention provides also an antibody, polynucleotide/vector, or pharmaceutical composition according to the invention for use as a medicament.

The invention provides also an antibody, peptide, polynucleotide/vector or pharmaceutical composition according to the invention for use in the treatment of diseases where TRPV6 channel is involved, in particular a disease associated with TRPV6 expression, such as a cancer.

The invention provides also an antibody, peptide, polynucleotide/vector or pharmaceutical composition according to the invention for the treatment of diseases where TRPV6 channel is involved, in particular a disease associated with TRPV6 expression, such as a cancer.

The invention provides also the use of an antibody, peptide, polynucleotide/vector according to the invention in the manufacture of a medicament for the treatment of diseases where TRPV6 channel is involved, in particular a disease associated with TRPV6 expression, such as a cancer.

The invention provides also a pharmaceutical composition for the treatment of diseases where TRPV6 channel is involved, in particular a disease associated with TRPV6 expression, such as a cancer comprising an antibody, peptide, polynucleotide/vector according to the invention as an active component.

The invention provides also a pharmaceutical composition comprising an antibody, peptide, polynucleotide/vector according to the invention for treating diseases where TRPV6 channel is involved, in particular a disease associated with TRPV6 expression, such as a cancer

As used herein, a disease associated with TRPV6 expression refers to a disease associated with altered TRPV6 expression, in particular TRPV6 overexpression.

The disease associated with TRPV6 expression may be selected from the group consisting of: cancer; skin diseases such as but not limited to psoriasis, alopecia and dermatitis, abnormal epithelial proliferation, skin aging, skin permeability, hyperphosphatemia and ectopic calcification); nerve/brain system disorders such as but not limited to deafness and post puberty goiter, preweaning, nerve excitability, estrous cycle and hypothalamus disorders, circadian rhythm, drug addiction, Parkinson's disease, pain sensation; digestive tract disorders such as but not limited to Crohn's disease, hypercalcemia, colonic crypt hyperplasia, intestinal bowel syndrome; Kidney diseases such as but not limited to calcification of arteries and kidney, chronic kidney disease; bone mineral density and osteoporosis diseases and disorders; Gynecological disorders such as but not limited to astrophoblast disorders, female infertility; and diabetes mellitus.

The cancer, including primary tumors and metastases thereof, is advantageously selected from the group consisting of: endometrial cancers, leukemia, and carcinomas of the breast, pancreas, prostate, colon, ovarian, and thyroid. In some more preferred embodiments said cancer is prostate cancer.

The invention provides also a method for treating a disease where TRPV6 channel is involved, in particular a disease associated with TRPV6 expression, such as a cancer, comprising: administering a therapeutically effective amount of the pharmaceutical composition according to the invention to the patient.

The pharmaceutical composition of the present invention is generally administered according to known procedures, at dosages and for periods of time effective to induce a beneficial effect in the individual. The administration may be by injection or by oral, sublingual, intranasal, rectal or vaginal administration, inhalation, or transdermal application. The injection may be subcutaneous, intramuscular, intravenous, intraperitoneal, intradermal or else.

The pharmaceutical composition of the invention is advantageously used in combination with surgery, radiotherapy, chemotherapy, and/or immunotherapy with immunomodulatory agents. The combined therapies may be separate, simultaneous, and/or sequential.

In some embodiments, the pharmaceutical composition is used for the treatment of humans.

In some embodiments, the composition of the invention is used for the therapeutic treatment of individuals which have been previously diagnosed with a disease where TRPV6 channel is involved, in particular a disease associated with expression of TRPV6, for example using an antibody according to the invention.

### Diagnostic and Prognostic Use of the Antibody

A subject of the present invention is also the use of the antibody according to the invention for the detection, diagnosing, prognosis of disease and/or treatment outcome of disorders where TRPV6 channel is involved, in particular diseases associated with TRPV6 expression, such as cancers.

In this connection, the present invention relates to the in vitro use of an antibody according to the invention, as diagnostic agent for the diagnosis of a disease where TRPV6 channel is involved, in a biological sample from an individual. The disease is in particular a disease associated with TRPV6 expression, such as cancer.

The present invention provides an in vitro method for the detection, diagnosis, prognosis of disease and/or treatment outcome, of a disease where TRPV6 channel is involved, in particular a disease associated with TRPV6 expression, such as cancer, in a biological sample obtained from an individual, comprising the steps of:
(a) incubating an antibody according to the invention with the biological sample to form a mixture; and
(b) detecting bound antibody in the mixture.

The antibody, preferably a labeled antibody as defined above, is used to detect TRPV6 protein expression in the individual, which is indicative of the individual having the disease, such as cancer.

For example, TRPV6 protein expression may be detected, in situ, in a tumor tissue from a patient, in comparison to the same type of tissue from a healthy individual.

In some embodiments, step (b) comprises the determination of the amount of bound antibody in the mixture, and optionally, comparing the amount of bound antibody in the mixture with at least one predetermined value.

In some embodiments, the method may comprise the step of deducing therefrom whether the individual is suffering from the disease.

The present invention also relates to the in vitro use of an antibody according to the invention, for detecting the presence of TRPV6 protein, or determining the amount of TRPV6 protein, present in a biological sample.

The present invention also relates to an in vitro method for detecting or determining the amount of TRPV6 protein in a biological sample, comprising the following steps:
a) bringing the biological sample into contact with an antibody according to the invention;
b) quantifying or detecting the presence or absence of bound antibody in the sample;
c) deducing therefrom the amount or the presence or absence of TRPV6 protein in the sample.

The present invention also relates to a method for diagnosis, prognosis of disease and/or treatment outcome, of a disease where TRPV6 channel is involved, in particular a disease associated with TRPV6 expression, such as cancer, in an individual, comprising the following steps:
a) administering an antibody according to the invention, preferably a labeled antibody to the individual; and
b) detecting, quantifying and/or localizing the antibody in the individual or a part of the individual.

In some embodiments, the method may comprise the step of deducing therefrom whether the individual is suffering from the disease.

The present invention also relates to a method for detecting, quantifying or localizing TRPV6 protein, in an individual or a part of an individual, comprising the following steps:
a) administering an antibody according to the invention to the individual;
b) detecting, quantifying and/or localizing the antibody in the individual or a part of the individual;
c) deducing therefrom the presence or absence, the amount and/or the localization of TRPV6 protein in the individual or the part of the individual.

When the antibody is administered to an individual, it is preferred for the antibody to be detectable by means of in vivo imaging methods, which are in particular external, such as planar or three-dimensional (3D) fluorescent imaging, or internal, such as endoscopy, for example.

The invention also relates to a method for evaluating the prognosis of a disease associated with TRPV6 expression such as cancer, in a biological sample obtained from an individual, comprising the steps of:
a) determining the level of TRPV6 protein in the biological sample using an antibody according to the invention, and
b) comparing the level in a) with a reference level for said protein, wherein if the level in a) is higher than said reference level, then said patient suffers from an invasive disease (cancer) with an unfavorable prognosis.

A reference value refers to a value established by statistical analysis of values obtained from a representative panel of individuals. The panel may for example depend from the nature of the sample, the type of disease. The reference value can for example be obtained by measuring TRPV6 protein expression level in a panel of normal individuals and/or individuals having a non-invasive disease such as non-invasive cancer and determining a threshold value, for example the median concentration, which is used as reference value. When the method according to the invention aims at monitoring a patient, the reference value may be obtained from the patient previously tested. Higher level refers to a significant higher level, i.e., p-value inferior to 0.1. The reference value is advantageously obtained from the same type of biological sample and/or from a panel of patients with the same type of disease, in particular the same type of cancer, as the tested patient.

The above mentioned prognosis method may further comprise, after the comparing step, a further step c) of classification of the patient(s) into favorable and unfavorable prognosis groups based on TRPV6 level(s) in said biological sample(s).

The above mentioned prognosis method may further comprise, after the classification step of the patients, a further step of administering an appropriate treatment to each group of patients depending upon the severity of the disease. The use of the prognosis method of the invention increases the efficiency of therapy of diseases associated with TRPV6 expression.

In particular, said patient is a newly diagnosed individual. Early evaluation of the prognosis of the cancer in the initial tumor of a patient using the method of the invention allows the choice of the most efficient therapy for the patient: local radiotherapy for a non-invasive tumor or systemic chemotherapy for an invasive tumor.

In the above methods and uses, a biological sample refers to a biological material obtained from the individual, which can be used in a detection or diagnostic assay. The biological material which may be derived from any biological source is removed from the patient by standard methods which are well-known to a person having ordinary skill in the art. The biological sample is advantageously biopsied tumor cells or tissue, or a body fluid such as serum, plasma, blood, lymph, synovial, pleural, peritoneal, or cerebrospinal fluid, mucus, bile, urine saliva, tears and sweat. In some embodiments, the biological sample is biopsied tumor cells or tissue.

In the above methods and uses, TRPV6 protein expression may be assayed directly on the biological sample or following a standard pretreatment, according to pretreatment methods which are well-known to a person having ordinary skill in the art. Pretreatment may include for example lysing cells, or embedding biopsied tissue in plastic or paraffin.

In the above methods and uses, TRPV6 protein expression may be detected or quantified using a variety of antibody-based techniques that are well-known to a person having ordinary skill in the art. Examples of such techniques include with no limitations immunoassays such as immunoblotting, immunoprecipitation, immunocytochemistry, immunohistochemistry, immunofluorescence like flow cytometry assays and FACS. Preferably, TRPV6 protein is detected or its level measured using an immunohistochemistry assay. One skilled in the art will know which parameters may need to be manipulated to optimize detection and/or quantification of the TRPV6 protein with anti-TRPV6 antibodies according to the invention, using these techniques.

The generated antibodies which are able to detect TRPV6 at the plasma membrane are useful for all in vitro or in vivo detection or diagnosis immunoassays on live, fixed or denatured cells or tissues.

In some embodiments of the above methods and uses for the detection, diagnosis or prognosis of a disease, said disease is a cancer as defined above including primary tumors and metastases thereof. The cancer is preferably selected from the group consisting of: endometrial cancers, leukemia, and carcinomas of the breast, pancreas, prostate, colon, ovarian, and thyroid. In some more preferred embodiments said cancer is prostate cancer.

In some embodiments of the above methods for the detection, diagnosis or prognosis of a disease, said patient is a human individual.

The above mentioned methods and uses for the detection, diagnosis or prognosis of a disease according to the present invention may be performed simultaneously or subsequently on biological samples from different patients. Expression levels of other biomarkers can be measured, in parallel.

Another subject of the present invention is a kit for detection, diagnosis or prognosis of a disease where TRPV6 channel is involved, in particular a disease associated with TRPV6 expression, such as cancer, comprising at least an antibody according to the invention, preferably a labeled antibody, and optionally instructions for the use of the antibody.

The practice of the present invention will employ, unless otherwise indicated, conventional techniques which are within the skill of the art. Such techniques are explained fully in the literature.

The invention will now be exemplified with the following examples, which are not limitative, with reference to the attached drawings in which:

### FIGURE LEGENDS

FIG. 1: Design and choice of epitope variants for different anti-TRPV6 channel antibodies raised against various peptide antigens

A. Scheme of the channel and relative positions of the epitopes bound by the four polyclonal antibodies, Ab79, Ab80 and Ab81 (base image of the channel from http://atlasgeneticsoncology.org/).

B. Scheme of the channel and relative positions of the epitope variants bound by the monoclonal antibody 82 (base image of the channel from http://atlas eneticsoncologv.org/).

FIG. 2: Schematic view of the phage display panning strategies

FIG. 3: Specificity ELISA of the 5 IgGs

Assessment of IgGs binding at high concentration to irrelevant peptide vs target peptides.

FIG. 4: Immunoblotting assay using anti-TRPV6 channel antibodies (Ab79 and 82) of invention and commercial antibodies

A. Immunoblotting of LNCaP cells (TRPV6-positive) treated either with 40 nM siRNA-Luciferase (siCT) or siRNA-TRPV6 (siV6) for 48 hours, following by whole cell lysates probed with the different anti-TRPV6 antibodies indicated on the bottom of the membrane.

B. The same PVDF membrane as in A, but probed with anti-beta actin antibody.

FIG. 5: Ab79a validation using knockdown/knockout and over-expression models

A. Immunoblotting of LNCaP, HEK, CHO, and PNT1A cell lines overexpressing TRPV6 channel using vEFlap-5′UTR-TRPV6_CMVp-mCherry vector (V6) compared to untransfected cells. Whole cell lysate was probed withAb79a.

B. Immunoblotting of PC3M, HAP-1 wt^{trpv6+/+}, HAP-1^{trpv6-/-} cell lines and BSA protein as compared to the housekeeping gene beta-actin (AKTB).

FIG. 6: Flow cytometry

The white histograms represent the secondary antibody alone, the light grey the tested antibody (Ab79, Ab82 or P3-R4-E11) plus secondary antibody.

FIG. 7: Effects of the antibody treatments on the calcium entry

A. Schematic diagram of store-operated capacitive calcium entry (SOCE) where TRPV6 channel takes an important part. The inhibition of SERCA pump with Thapsigargin, provokes calcium leak which will empty calcium stores and thus activate store-operated channels (SOC), which, in turn, will activate TRPV6 channel taking an important part in the amplification of the calcium entry inside the cells.

B. Quantitative representation of SOCE into LNCaP cells pretreated 5 min with either glycerol (CT), or rabbit polyclonal anti-HA or polyclonal anti-TRPV6 antibodies No:79 (79a).

C. Quantitative representation of SOCE into LNCaP cells submitted to anti-TRPV6 antibody No: 79 (79a) pretreatment for 5 min 79, with the knockdown of TRPV6 channel (siRNA, 40 nM, 48 hours).

D. Capacitive entry of calcium into a prostate cancer cell line LNCaP WT (Curve 1), pretreated with Ab83 (Curve 2) and pretreated with Ab79 (Curve 3).

FIG. 8: Antibody alter the electrical currents passing through the TRPV6 channel

A. Quantitative representation of the TRPV6-specific currents from HEK cell transfected with vEFlap-5′UTR-TRPV6 wt CMVp-mCherry vector and treated with rabbit polyclonal anti-TRPV6 antibody No:79 (79a) as compared to control rabbit polyclonal anti-HA epitope antibody of the same isotype. n=3, *—p<0.05.

B. Dose-response curves of 0.5 μg/μl of the above rabbit polyclonal anti-TRPV6 antibody No:79(79a) in different dilutions.

C. Quantitative representation of the TRPV6-specific currents from HEK cell transfected with vEFlap-5′UTR-TRPV6 wt CMVp-mCherry vector and treated with mouse monoclonal anti-TRPV6 antibody No:82. The curves represent typical currents before or after stimulation of TRPV6 activity by DFV solution, as well as following the application of 1:5000, 1:2000, 1:1000, 1:500 and 1:200 dilutions of the antibody, as indicated. N equals: 1:5000 (n=9), 1:2000 (n=11), 1:1000 (n=13), 1:500 (n=25) and 1:200 (n=13), *—p<0.05.

FIG. 9: TRPV6 modulation via polyclonal antibodies 79 (79a) binding decreases cell survival

A. Cell survival assay (MTS) of LNCaP cells treated either with equivalent quantity of glycerol as control (CT), anti-TRPV6 antibody No:79(79a) or control antibody anti-HA, for 3 days. Dilutions are normalized to the initial quantity of 0.5 μg/μl, n=3, *—p<0.05; **—p<0.01.

B. Cell count of LNCaP cells treated for 3 days with polyclonal anti-TRPV6 antibody No:79(79a) and also antibody No:80 and 81 (raised against intracellular epitopes of TRPV6 channel), anti-SERCA2B antibody of the same isotype and glycerol (CT); n=3, **—p<0.01.

C. Cell survival assay (MTS) of LNCaP cells treated either with medium, equivalent quantity of glycerol as control (CT), anti-TRPV6 antibody No:79(79a) or commercial anti-TRPV6 antibody (Alomone #ACC-036) for 3 days. Dilutions are normalized to the initial quantity of 0.5 μg/μl, n=3, *—p<0.05; **—p<0.01.

FIG. 10: Treatment of prostate cancer cells by polyclonal antibodies 79 (79a) induces apoptosis of prostate cancer cells

A. Quantification of apoptosis rate assay using Hoechst staining of LNCaP cells. Cells were pretreated for 72 hours either with the equivalent quantity of glycerol (CT) or polyclonal anti-TRPV6 antibody No:79(79a) for 72 hours (1/500, 0.5 μg/μl). Treatment with 1 μM of Thapsigargin (TG) for 72 hours was used as a positive control to induce apoptosis. n=3,**—p<0.01, ***—p<0.001. § —p<0.05 as compared to TG (1 μM, 72 hours) only treatment.

B. Quantification of apoptosis rate using Hoechst staining of HEK cells. n=3, **—p<0.01, ***—p<0.001. § —p<0.05 as compared to TG (1 μM, 72 hours) only treatment.

C. Quantification of sub-G1 peak of the cell cycle assay of LNCaP cells treated for 72 hours either with equivalent quantity of glycerol (CT) or polyclonal anti-TRPV6 antibody No:79(79a) for 72 hours (1/500), or anti-HA of the same isotype. Treatment with 1 μM of the Thapsigargin (TG) for 72 hours was used as a positive control to induce apoptosis. n=3, * —p<0.05; **—p<0.01.

D. Trypan blue staining of LNCaP cells treated with equivalent quantity of glycerol (CT) or polyclonal anti-TRPV6 antibody No:79 (79a) for 72 hours (1/500), or anti-HA of the same isotype, carried out during 8, 24 and 48 hours. n=3, *—p<0.05; **—p<0.01.

FIG. 11: LNCaP prostate cancer cells Survival

Cells were treated for 1, 2, 3 and 4 days with either Glycerol or mabAU1, the control antibody, or with mab82. n=3; *—p<0.05.

FIG. 12: Effects of the mouse monoclonal antibody mab82 (82a) on tumor growth and metastasis progression in immunodeficient mice in vivo

Mice were grafted with 2×10E6 cells from the stable clones of PC3M^{trv6-/-}—pmCherry and PC3M^{trPvP-/-}—pTRPV6 wt cell lines. Each mouse was grafted at the neck and the back levels, and each group was divided by two for the treatment with either control anti-AU1 or experimental mab82 (82a) antibody at the same dose of 100 μg/kg.

A. Tumor growth in mm³ measured every 3^{rd} day in PC3M^{trpv-+-+}—pTRPV6 wt group control anti-AU1 or experimental mab82 (82a) antibody subgroups. Arrow denotes the beginning of treatments. *—p<0.05; **—p<0.001.

B. Survival curve of the mice from both groups

C. Tumor growth in mm³ measured every 3^{rd} day in PC3M^{trpv6+/+}-mCherry group control anti-AU1 or experimental mab82 (82a) antibody subgroups. Arrow denotes the beginning of treatments.

D. Tumor growth in mm³ measured every 3^{rd} day in PC3M^{trpv6+/+}-mCherry group versus PC3M^{trpv6-/-}-pTRPV6wtanti-AU1 subgroups. Arrow denotes the beginning of treatments. *—p<0.05.

E. Metastasis occurrence in % between PC3M^{trpv6-/-}—pmCherry and PC3M^{trpv6-/-}-pTRPV6 wt groups and both subgroups: control anti-AU1 or experimental mab82 (82a) antibodies.

FIG. 13: Effects of the antibody treatments on the calcium entry

Capacitive entry of calcium into a prostate cancer cell line LNCaP WT pretreated with P3R4F03, pretreated with P3R4E11, pretreated with P3R5H03 and with no pretreatment (CT).

FIG. 14: Effects of the antibody treatments on the calcium entry

Capacitive entry of calcium into a prostate cancer cell line LNCaP WT pretreated with humanized mab82, pretreated with murine mab82, pretreated with P2R4G08 and with no pretreatment (CT).

FIG. 15: TRPV6 modulation via P3R4F03 antibody binding decreases cell survival

Cell survival assay (Cell titer glo) of LNCaP cells treated either with equivalent quantity of Human IgG1 Irrelevant antibody as control (IA), anti-P3R4F03 antibody for 3 days. Dilutions are normalized to the initial quantity of 0.5 μg/μl, n=3, * p<0.05; ** p<0.01; *** p<0,001.

### EXAMPLES

### Material and Methods

### Peptide Epitopes

Peptide epitopes (peptide antigens) are derived from human TRPV6 sequence UniProtKB/Swiss-Prot: NP_061116.5 or Q9H1D0.3 (SEQ ID NO: 1). Peptides were synthesized and purified with >99% purity.

### Polyclonal Antibody Production

The peptide epitope (peptide antigen) was coupled to a KLH protein to its N-terminus and injected to the rabbits once per week during four weeks following by the final bleed (Eurogentec, LTD). The serum was tested in ELISA using antigen coated plates following by affinity purification in columns against the same bound antigen. Final affinity-purified antibodies were supplied, diluted 50/50 v/v with the glycerol and stored at −20° C.

### Monoclonal Antibody Production, Cloning and Characterization

The manufacturing process is a standard one consisting of the 4 consequent immunizations with the same peptide antigen. 1 week after the 4th boost, the samples of serum were tested and the right antibody-bearing animal was chosen, sacrified, and its B-lymphocytes were fused with hybridomas. Once the sufficient titer was obtained, the samples of mAb were tested one more time to choose the most efficient/expected one. Then, the hybridomas were multiplied, and antibody was isolated and affinity purified against the epitope peptide on the columns.

Primers and siRNAs

TABLE II
Primers for qPCR and siRNAs

| Name, | | | |
|---|---|---|---|
| Accession | Forward | Reverse | Size |
| No | (5′-...-3′) | (5′-...-3′) | (b.p) |
| TRPV6 | CCCAAGGAGAAAGGGCTAAT | TTGGCAGCTAGAAGGAGAGG | 145 |
| NM_018646 | (SEQ ID NO: 190) | (SEQ ID NO: 191) | |
| HPRT | GGCGTCGTGATTAGTGATGAT | CGAGCAAGACGTTCAGTCCT | 134 |
| NM_000194 | (SEQ ID NO: 192) | (SEQ ID NO: 193) | |

| | |
|---|---|
| TRPV6 | 5′-CCUGCUGCAGCAGAAGAGG (dTdT)-3′ (SEQ ID NO: 194) |
| siRNA-1 | |
| TRPV6 | 5′-GACUCUCUAUGACCUCACA (dTdT)-3 (SEQ ID NO: 195) |
| siRNA-2 | |
| TRPV6 | 5′-CGUCAUGUACUUCGCCCGA (dTdT)-3′ (SEQ ID NO: 196) |
| siRNA-3 | |
| TRPV6 | 5′-CCUCCUCAUUGCCAUGAUG (dTdT)-3′ (SEQ ID NO: 197) |
| siRNA-4 | |
| siLuciferase, | 5′-CUUACGCCUGAGUACUUCGA(dTdT)-3′ (SEQ ID NO: 198) |
| AB_490793 | |

### Reagents

All reagents were purchased from Sigma (Sigma, L'Isle d'Abeau Chesnes, France) unless otherwise specified.

### Cell Culture

Human PC3M (metastatic cell line derived from PC3 cells grafted in vivo), PC-3M, LNCaP, PNTA1, HEK293 and CHO-K1 cell lines were from American Type Culture Collection (ATCC) and were cultured in RPMI (LNCaP, PC3M, PNT1A), DMEM (HEK293) and F12 (CHO) media (Gibco-BRL) supplemented with 10% foetal calf serum and containing kanamycin (100 μg/ml) and L-glutamine (2 mM) where necessary. HAP1 cell line is a near-haploid human cell line that was derived from the male chronic myelogenous leukemia (CML) cell line KBM-7 (Carette et al. Nature. 2011, 477, 7364, 340-3), and cultured in IMDM medium (Sigma-Aldrich) supplemented with 10% foetal calf serum and containing kanamycin (100 μg/ml) and 1-glutamine (2 mM). PNTla cell line was from from American Type Culture Collection (ATCC) and was cultured in RPMI. All the cells were cultured at 37° C. in a humidified atmosphere with 5% CO₂ in air. The medium was changed three times a week and cultures were split by treating the cells with 0.25% trypsin (in PBS) for 5 min at 37° C. before reaching confluence. For the experiments, cells were seeded in 6-well plates for PCR and western-blotting. To maintain trpv6^{-/-} status of the cells, the antibiotic of selection G418 was used at the concentration of 200 μg/ml for the maintenance in culture of the HAP1^{trpv6-/-} cells, and puromycin at 0.1 μg/ml for the PC3M^{trpv6-/-} cell line.

For the antibodies treatments, the serum was descomplemented very thoroughly, i.e. heated at 62° C. for 1 hour with the permanent agitation, or in some cases, serum-free medium such as AIM V from Gibco™ was used.

### Electrophysiology and Solutions

Macroscopic currents were recorded from HEK-293 cells transfected with vEFlap-5′UTR-TRPV6_CMVp-mCherryvector in the whole-cell configuration of the patch-clamp technique using a computer controlled EPC-9 amplifier (HEKA Electronic, Germany), as previously described (Raphaël et al. 2014). The composition of the extracellular solution for patch-clamp recording was (in mM): 120 NaCl, 5 KCl, 10 CaCl₂, 2 MgCl₂, 5 glucose, 10 HEPES, pH 7.4 adjusted with TEA-OH, osmolarity 310 mOsm/kg adjusted with D-Mannitol. The patch pipettes were filled with the basic intracellular pipette solution (in mM): 120 Cs-methane sulfonate, 10 CsCl, 10 HEPES, 10 BAPTA (1.2-bis(2-amonophenoxy)ethane N,N,N′,N′tetraacetic acid), 6 MgCl₂ (pH adjusted to 7.4 with CsOH and osmolarity 295 mOsm/kg adjusted with D-Mannitol). The necessary supplements in the desired concentrations were added to the experimental solutions directly from appropriate stock solutions, dissolved in water, ethanol or dimethylsulfoxide. All chemicals were purchased from Sigma-Aldrich. In the course of patch-clamp recording drugs and solutions were applied to the cells via multiline microperfusion system with common outflow (Cell Micro Controls, Norfolk, VA) placed in the close proximity (˜200 m) to the studied cell. Experiments were carried out at room temperature.

### Calcium Imaging

Cells were plated onto glass coverslips and were loaded with 4 μM Fura-2 AM at room temperature for 45 min in the growth medium. Recordings were performed in HBSS containing (in mM): 140 NaCl, 5 KCl, 2 MgCl₂, 0.3 Na₂HPO₃, 0.4 KH₂PO₄, 4 NaHCO₃, 5 glucose and 10 HEPES adjusted to pH 7.4 with NaOH. CaCl₂ was adjusted to 0.07 mM or 1.8 mM depending on the experiment. The coverslips were then placed in a perfusion chamber on the stage of the microscope. Fluorescence images of the cells were recorded with a video image analysis system (Quanticell). The Fura-2 fluorescence, at the emission wavelength of 510 nm, was recorded by exciting the probe alternatively at 340 and 380 nm. The signal ratio at 340/380 nm was converted into [Ca²⁺ ]ᵢ level using an in vitro calibration.

### SDS-PAGE and Western-Blotting

Semiconfluent cells were treated with an ice-cold lysis buffer containing: 10 mMTris-HCl, pH 7.4, 150 mMNaCl, 10 mMMgCl, 1 mM PMSF, 1% Nonidet P-40, and protease inhibitor cocktail from Sigma. The lysates were centrifuged 15,000×g at 4° C. for 20 minutes, mixed with a sample buffer containing: 125 mMTris-HCl pH 6.8, 4% SDS, 5% β-mercaptoethanol, 20% glycerol, 0.01% bromphenol blue, and boiled for 5 min at 95° C. Total protein samples were subjected to 8, 10, and 15% SDS-PAGE and transferred to a nitrocellulose membrane by semi-dry Western blotting (Bio-Rad Laboratories). The membrane was blocked in a 5% milk containing TNT buffer (Tris-HCl, pH 7.5, 140 mMNaCl, and 0.05% Tween 20) overnight then probed using specific rabbit polyclonal anti-TRPV6 antibodies (all at 1/500 dilution) and mouse monoclonal anti-3-actin (Lab Vision Co., 1/1000) antibodies. The bands on the membrane were visualized using enhanced chemiluminescence method (Pierce Biotechnologies Inc.). Densitometric analysis was performed using a Bio-Rad image acquisition system (Bio-Rad Laboratories).

### Flow Cytometry

200 000 cells were incubated with polyclonal rabbit antibody pAb 79 or monoclonal mouse antibody mAb 82 at 15 μg/mL for one hour on ice. IgG binding was detected using anti-rabbit-AF488 or anti-mouse-AF488 antibodies (Invitrogen A-11034 and A-11029, respectively). or with P3-R4-E11 at 10 μg/mL for one hour on ice. IgG binding was detected using an anti-Fab-AF647 (Jackson-109-605-006). Analysis was realised by flow cytometry.

### RT-PCR

RT-PCR experiments were performed as previously described (Lehen'kyi et al. 2007). Total RNA was isolated using the guanidium thiocyanate-phenol-chloroform extraction procedure. After DNase I (Life Technologies) treatment to eliminate genomic DNA, 2 μg of total RNA was reverse transcribed into cDNA at 42° C. using random hexamer primers (Perkin Elmer) and MuLV reverse transcriptase (Perkin Elmer) in a 20 μl final volume, followed by PCR as described below. The PCR primers used to amplify TRPV6 cDNAs as well as the primers for AR, VDR, and 3-actin are specified in Table XVI above. PCR was performed on the RT-generated cDNA using a GeneAmp PCR System 2400 thermal cycler (Perkin Elmer). To detect different cDNAs, PCR was performed by adding 1 μl of the RT template to a mixture of (final concentrations): 50 mMKCl, 10 mMTris-HCl (pH 8.3), 2.5 mM MgCl2, 200 μM of each dNTP, 600 nM of sense and antisense primers, and 1 U AmpliTaq Gold (Perkin Elmer) in a final volume of 25 μl. DNA amplification conditions included the initial denaturation step of 7 min at 95° C., and 40 cycles of 30 s at 95° C., 30 s at 60° C., 30 sec at 72° C., and finally 7 min at 72° C. Primers used are listed in Table above.

### Quantitative Real-Time PCR

The quantitative real-time PCR of TRPV6 and HPRT mRNA transcripts was done using MESA GREEN qPCR MasterMix Plus for SYBR Assay (Eurogentec) on the Biorad CFX96 Real-Time PCR Detection System. The sequences of primers are indicated in Table XVI. The HPRT gene was used as an endogenous control to normalize variations in the RNA extractions, the degree of RNA degradation, and variability in RT efficiency. To quantify the results, the comparative threshold cycle method AACt and Biorad CFX Manager Software v2.0.

siRNA Transfection

HAP1 cells were transfected with 40 nM of siRNA against TRPV6 (1-4 or mix) or siLuciferase (Eurogentec, LTD, Belgium) using 5 μl de Lipofectamine 3000 transfection reagent (Lipofectamine 3000, Thermofisher) following the manufacturer's instructions (see Table I for the siRNA sequences). The efficiency of cell transfections with the siRNAs for each particular target has been validated using real-time quantitative PCR and/or western-blotting where appropriate.

### Nucleofection

Transfection of various cell lines with different plasmids was carried out using Nucleofector (Amaxa GmbH) according to the manufacturer's instructions. Briefly, 2 μg of the plasmid was transfected into 2 millions of trypsinized cells, which then were plated onto six-well dishes, 35 mm dishes or onto the glass coverslips for 48 hours.

### Cell Survival Assay

Cell proliferation was measured using the CellTiter 96 Aqueous One Solution cell proliferation assay (Promega), on the basis of the cellular conversion of the colorimetric reagent MTS [3,4-(5-dimethylthiazol-2-yl)-5-(3-carboxymethoxyphenyl)-2-(4-sulfophenyl)-2H-tetrazolium salt] into soluble formazan by dehydrogenase enzymes found only in metabolically active, proliferating cells. Following each treatment, 20 μl of dye solution was added into each well in 96-well plate and incubated for 2 h. Subsequently, absorbance was recorded at 490 nm wavelength using an ELISA plate reader (Molecular Devices). Cellular proliferation inhibition rate is calculated as: (A_{control}−Aₛₐₘₚₗₑ)/(A_{control}−A_{blank})×100%.

To evaluate P3R4F03, cell survival was measured using the CellTiter-Glo® Luminescent Cell Viability Assay, on the basis of the firefly luciferase reaction “to transform luciferin to light with the ATP produce by metabolically active cells, proliferating cells. Cells were treated with either irrelevant antibody (IA) or P3R4F03 at equal dose. Following each treatment, 100 μl of CellTiter-Glo® Reagent solution was added into each well in 96-well plate and incubated for 10 min Subsequently, luminescence was recorded using an ELISA plate reader (Polar Star Omega-BMG Labtech-germany). Cellular survival inhibition rate is calculated as: (Acontrol−Asample)/(Acontrol−Ablank)×100%.

### Cell Cycle Assay

Flow cytometry assays were performed on cell populations cultured in triplicate 25-cm² flasks as originally described (18). Approximately 10⁶ cells were fixed with 1 ml ice-cold 70% methanol for 30 min. After fixing, cells were pelleted by centrifugation to remove the fixatives, washed three times with phosphate-buffered saline (PBS) at 4° C., resuspended in 100 μl PBS, treated with 100 μl RNAse A (1 mg/ml, Sigma), and stained with propidium iodide (PI, Sigma) at a final concentration of 50 μg/ml. The stained cells were stored at 4° C. in the dark and analyzed within 2 h. The stained samples were measured on a FACS can flow cytometer (Becton-Dickinson, San Jose, CA). Data were acquired for 7000 events with a variation coefficient of less than 5%, and red fluorescence was measured using a fluorescence detector 3 (FL3) on the X-axis. The data were stored and analyzed using CellQuest software to assess cell-cycle distribution patterns (subG1 (apoptotic), G0/G1, S, and G2/M phases).

### TUNEL Assay

The level of apoptosis was estimated from the number of apoptotic nuclei revealed either by TUNEL-TMR red assay (Roche Biochemicals) or by Hoechst staining. The percentage of apoptotic cells was determined by counting at least five random fields for each condition done in triplicate for each “n”.

### Immunohistochemistry

Paraffinized human prostate anonymous tissue sections from 18 prostatectomies were obtained from the Department of Cell Pathology, Hôpital St Vincent de Lille. Once excised tumors were fixed and paraffinized according to conventional procedure following by microtome cut at 7 μm and stacked to the slides. Paraffin-embedded prostate sections were subjected to conventional deparaffinization followed by antigen retrieval using citrate buffer at 95° C. in water bath. After saturation in the solution containing 1% BSA and 0.05% Triton X100 in PBS-gelatin, the prostate sections were incubated with the specific antibodies, such as rabbit polyclonal anti-TRPV6 antibodies (No:79a-c, 80-82, 1/200), overnight at 4° C. Donkey or Goat polyclonal anti-rabbit and anti-mouse peroxidase-conjugated secondary antibodies (Chemicon International; 1/200) were used. After revelation with diaminobenzidine (Sigma-Aldrich), slides were covered with Glycergel®, and images were analysed using Zeiss Axioskope microscope (Carl Zeiss) and Leica Image Manager software (Leica Geosystems AG). Immunohistochemistry was performed automatically using a Benchmark XT automated slides stainer (Ventana Medical Systems, Inc., Tucson, AZ) following established protocols and detection was performed using an IVIEW-DAB detection system (N760-500, Ventana Medical Systems, Inc.).

### Plasmids

The whole TRPV6 cDNA containing 5′-UTR on the pCAGGS was used to obtain a final vEFlap-5′UTR-TRPV6_CMVp-mCherryvector (E-Zyvec, France) which was nucleofected into the cells and the transfection rate was evaluated using a control vEFlap-5′UTR_CMVp-mCherry vector. pTRPV6-eYFP and pOrail-YFP vectors were used as previously described (Raphaël et al. 2014).

### Animals, Antibody Injections, Tumorigenicity Assays, and Surgery

Studies involving animals, including housing and care, method of euthanasia and experimental protocols were conducted in accordance with the animal ethical committee (approval No: 201703021400830) in the animal house (permit: C59-00913) of the University of Lille (campus Cite Scientifique), under the supervision of Dr. Lehen'kyi (permit: 59-009270). Tumour cells (2×10⁶ cells/mouse) were injected subcutaneously in 50% (v:v) matrigel (BD biosciences) into 6-weeks old male swiss nude mice (Charles-Rivers, France). In the antibody studies, once the tumors start to be visible, mice were randomized for treatment (at least 10 animals/group) and received twice per week intraperitoneally either anti-AU1 or anti-TRPV6 mab82 antibodies, 100 μg/kg diluted in PBS. Mice were sacrificed if the well-being of the animal was violated. Once the tumor reached the maximal authorized size, an animal was subjected to surgery, i.e. tumor excised and. Tumors were dissected, photographed, weighed and volume was got. For the metastasis studies, animals were monitored daily and mCherry imaging was done using small animal imaging system (Bruker, USA).

scFv Screening

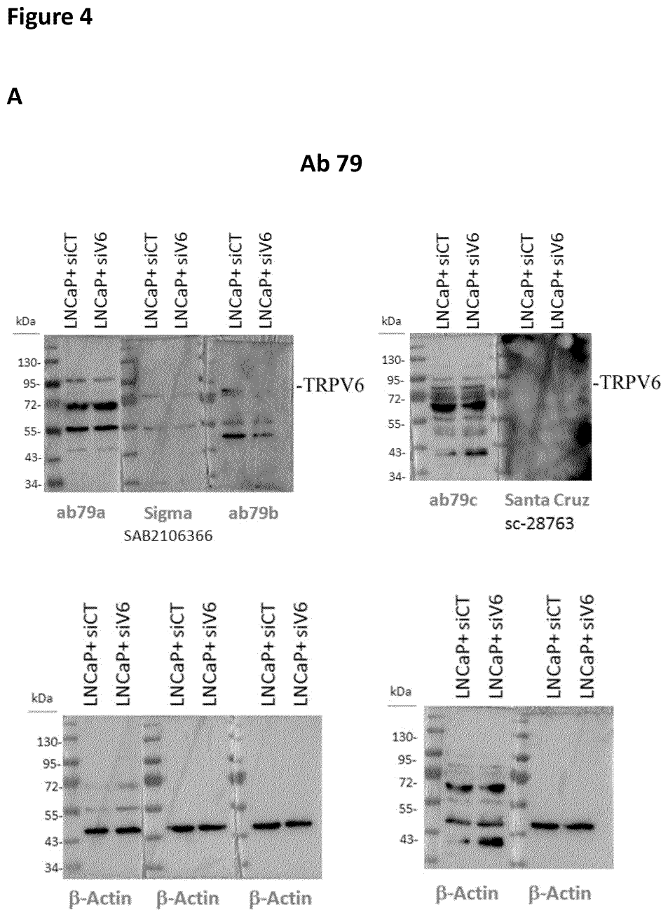

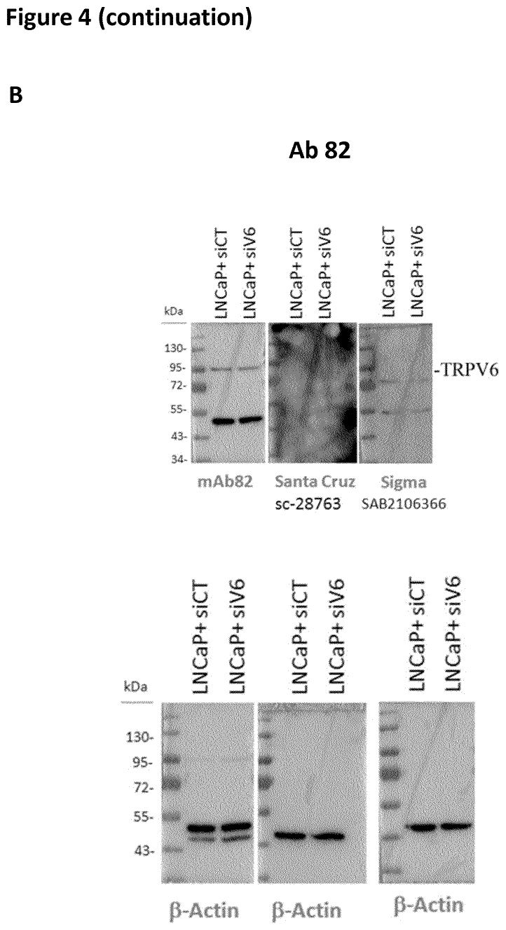

Three phage display selection strategies were carried out using iMAb's HuscI II proprietary library, on TRPV6 peptides and PC3M luc C6 cells (FIG. 18). Depletion was carried out on streptavidin as described in FIG. 4. Depleted library has been used to perform 4 first rounds of selection on peptides and a fifth one on cells.

For the 2 first round, Biotinylated-TRPV6 peptides were immobilized on streptavidin coated maxisorp plates. Phages (10¹⁰ phages/mL) from each round of selection were added and detected using anti-M13 antibody-HRP.

Selected scFv from 2 first round expression was induced and culture supernatants containing the secreted scFv were collected to assess scFv binding to TRPV6 peptides by ELISA for round 3, 4 and 5.

For round 3, 4, 5 Biotinylated peptides were immobilized on streptavidin coated maxisorp plates. ScFv productions (culture supernatants) were added and detected using anti-c-myc-HRP.

### SDS-PAGE Analysis of Purified IgGs

Proteins were reduced or not and 1.6 g of protein were loaded per lane on a 4-20% gel.

### Dose-Response ELISA

TRPV6 biotinylated peptides were immobilized at 10 μg/mL on a streptavidin coated plate. IgGs binding was tested at different concentrations (from 0.00282 to 500 nM) and detected using an anti-Fab-HRP (Sigma A0293).

### Specificity ELISA

TRPV6 and irrelevant biotinylated peptides were immobilized on streptavidin coated plates. IgGs binding was tested at high concentration (75 μg/mL-500 nM) and detected using an anti-Fab-HRP (Sigma A0293).

### Data Analysis

For each type of experiment the data were accumulated from at least three measurements. Data were analyzed using Origin 7.0 (Microcal Software Inc., Northampton, MA) software. Results were expressed as Mean±S.E.M., where appropriate. N equals to the number of series of experiments, n equals to the number of cell used in the study. ANOVA was used for statistical comparison of the differences and P<0.05 was considered significant. In the graphs, (*) and (**) denote statistically significant differences with P<0.05 and P<0.01, respectively.

### Example 1: Design and Validation of Anti-TRPV6 Antibodies Raised Against Extracellular Epitopes

### Choice of the Epitopes for the Rabbit Polyclonal Antibodies No. 79

37 amino acids span the first extracellular loop situated between S1 and S2 transmembrane domains. Of them 3 residues are those of Asparagin, N. A detailed analysis using NetNGlyc 1.0 software demonstrated the most probable second and third sites of N-glycosylation, RTNNRT and RDNTL. The presence of these sites and the N-glycosylation thereof will deny the potential steric access to the epitope by the antibody. From the other side, the lipid bilayer will preclude the antibody from binding to the respective amino acids of the epitope. Three epitopes were used to generate rabbit polyclonal antibodies called 79a-c. These three epitopes correspond to the peptide 79a or hTRPV6 414-428; positions 414 to 428 of hTRPV6 sequence SEQ ID NO: 1; QEAYMTPKDDIRLVG (SEQ ID NO: 3); the peptide 79b or hTRPV6 414-425; positions 414 to 425 of hTRPV6 sequence SEQ ID NO: 1; QEAYMTPKDDIR (SEQ ID NO: 4); and the peptide 79c or hTRPV6 412-425; positions 412 to 425 of hTRPV6 sequence SEQ ID NO: 1; LLQEAYMTPKDDIR (SEQ ID NO: 5), respectively. Their efficiency to retrieve the antigens was demonstrated using a series of immunoblottings in denaturing conditions.

### Monoclonal Antibody 83

One monoclonal antibody was raised against peptide EAYMTPKEEIRR (SEQ ID NO: 7) which is a variant of peptide hTRPV6 415-426 (EAYMTPKDDIRL; SEQ ID NO: 8) situated at the C-terminus end of the X-loop between S1 and S2 transmembrane domains.

### Choice of the Epitopes for the Monoclonal Antibody 82

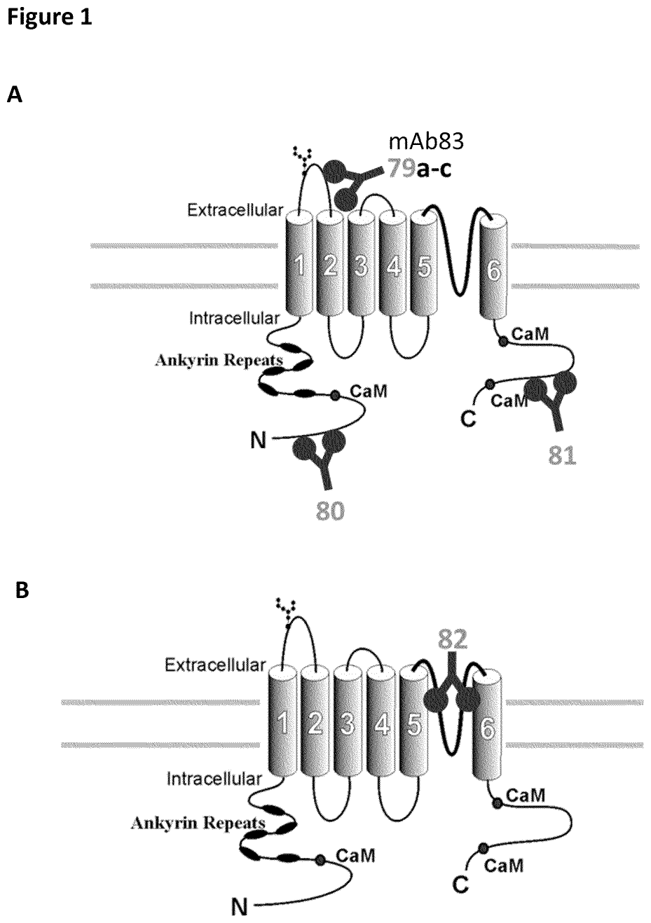

One monoclonal antibody was raised against peptide situated at the N-terminus end of the p-loop (between S5 and S6) in the pore region (FIG. 1). Various antigen peptides (peptide epitopes) situated in the target sequence hPRV6 551-573 (IFQTEDPEELGHFYDYPMALFST; SEQ ID NO: 9) were tested: peptide 82a (hPRV6 553-570; QTEDPEELGHFYDYPMAL; SEQ ID NO: 10); peptide 82b (hPRV6 551-567; IFQTEDPEELGHFYDYP; SEQ ID NO: 11); peptide 82c (hPRV6 557-573; PEELGHFYDYPMALFST; SEQ ID NO: 12); peptide 82d (hPRV6 554-568; TEDPEELGHFYDYPM, SEQ ID NO: 13); peptide 82 (hPRV6 554-569; TEDPEELGHFYDYPMA; SEQ ID NO: 14) Monoclonal antibody 82 (mab82) raised against peptide 82 (hPRV6 554-569) was further characterized.

### Design and Validation of Anti-TRPV6 Antibodies Raised Against Extracellular Epitopes by Phage Display

Three peptides of human TRPV6 were synthesized by Genosphere Biotechnologies. One was biotinylated at its N-terminus and the 2 others at their C-terminus. Their main characteristics are summarized in Table III.

TABLE III
Peptides characteristics

| Peptide | Sequence | MW |
|---|---|---|
| Peptide 1 | QEAYMTPKDDIRLVGK-[biot] | 2108.46 Da |
| (Loop 1) | (SEQ ID NO: 6) | |
| Peptide 2 | TEDPEELGHFYDYPMAK-[biot] | 2286.51 Da |
| (Pore Forming 1) | (SEQ ID NO: 15) | |
| Peptide 3 | [biot]-[C6spacer]-DGPANYNVDLPFMYS | 2223.29 Da |
| (Pore Forming 2) | (SEQ ID NO: 16) | |

Peptides 1 and 2 were those previously used for peptide immunisation to discover respectively pAb79 and mAb82 whereas peptide 3 is an additional peptide that was designed to target another loop of the pore forming region.

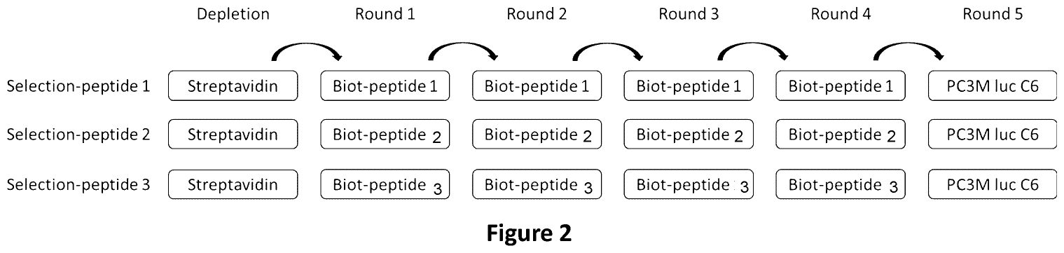

Three phage display selection strategies were carried out using iMAb's HuscI II proprietary library, on TRPV6 peptides and PC3M luc C6 cells (FIG. 2). Depletion was carried out on streptavidin as shown in FIG. 2. Depleted library has been used to perform 4 first rounds of selection on peptides and a fifth one on cells. After the first 3 rounds of selection, the results of the 3 panning strategies were tested for binding to TRPV6 peptides by polyclonal ELISA using the phage pools selected at each panning round and an anti-M13-HRP as a secondary antibody. An enrichment in TRPV6 peptide binders was observed for all selections. After the 3 first rounds of selection, 93 individual colonies (containing a single scFv) of each 3 selections were picked and grown. scFv expression was induced and culture supernatants containing the secreted scFv were collected to assess scFv binding to TRPV6 peptides by ELISA scFvs were detected using anti-c-myc-HRP antibody. Altogether, 21 clones were found positive at the end of round 3: 3 on peptide 1, 7 on peptide 2 and 11 on peptide 3. In order to further enrich the selections in TRPV6 binders, it was decided to perform 2 extra rounds, on peptides and on cells respectively. The selection outputs of round 4 and 5 were screened as previously described.

Altogether, 22 clones were found positive at the end of round 4: 6 on peptide 1, 7 on peptide 2 and 9 on peptide 3.

Altogether, 4 clones were found positive at the end of round 5: 2 on peptide 1, and 2 on peptide 3.

After screening of round 3, 4 and 5, 47 scFvs have shown to bind to TRPV6 peptides specifically.

Forty-seven scFvs were selected by ELISA screening and sent for sequencing. Thirty-four of them were unique sequences: 8 for peptide 1, 11 for peptide 2 and 15 for peptide 3.

Sequences with high sequence identity were grouped in clusters. An additional ELISA was carried out to confirm specific binding of unique clones. ScFv expression was induced and culture supernatants were collected to assess scFv binding to TRPV6 peptides and to irrelevant peptide in triplicate. ScFvs were detected using anti-c-myc-HRP antibody. 11 of ScFvs were selected for further characterization in IgG format: The 11 selected scFvs were subcloned into Human IgG1 format, expressed at small-scale in HEK293T cells and purified on protein A beads. The SDS-PAGE migration profile of 9 out of 11 were comparable to control IgG (Trastuzumab). Two IgGs showed a higher molecular weight, corresponding to N-glycosylation which was confirmed by sequence analysis (data not shown).

IgGs binding to Human TRPV6 peptides was tested by ELISA. TRPV6 biotinylated peptides were immobilized at 10 μg/mL on a streptavidin coated plate. IgGs binding was tested at different concentrations (from 0.00282 to 500 nM) and detected using an anti-Fab-HRP (Sigma A0293). IgGs P2-R4-G8, P3-R4-F3, P3-R4-E11, P3-R5-E6 and P3-R5-H3 showed a strong binding to TRPV6 peptides, allowing EC50 determination (Table IV).

TABLE IV
EC50 values of the 11 IgGs

| IgG | EC50 (nM) | R² |
|---|---|---|
| P2-R4-G8 | 0.49 | 0.9958 |
| P3-R4-F3 | 0.86 | 0.9948 |
| P3-R4-E11 | 0.90 | 0.9991 |
| P3-R5-E6 | 0.98 | 0.9941 |
| P3-R5-H3 | 0.36 | 0.9891 |

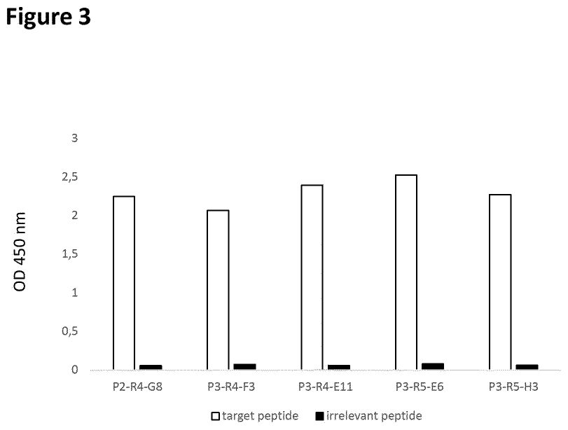

Next, the IgGs specificity was assessed by ELISA. TRPV6 and irrelevant biotinylated peptides were immobilized on streptavidin coated plates. IgGs binding was tested at high concentration (75 μg/mL-500 nM) and detected using an anti-Fab-HRP (Sigma A0293). No binding to irrelevant peptide was observed for IgGs P2-R4-G8, P3-R4-F3, P3-R4-E11, P3-R5-E6 and P3-R5-H3, showing a high specificity towards the peptide they have been selected on (FIG. 3).

### Detection of TRPV6 Protein Expression Using Different Rabbit Polyclonal Antibodies

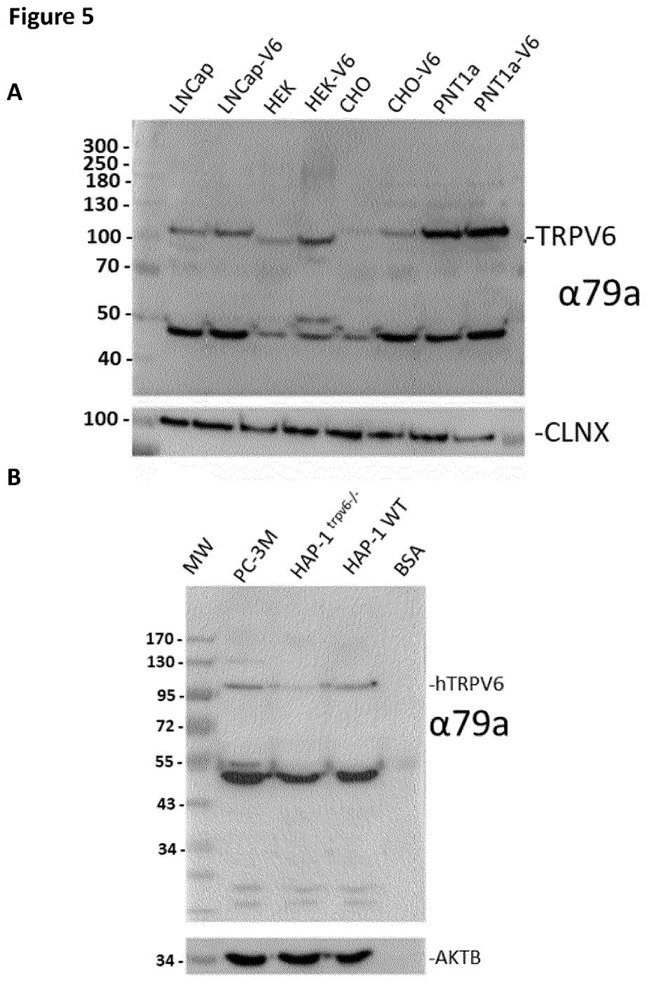

Three polyclonal antibodies (Ab79a, b, c) were raised against peptides situated at the C-terminus end of the X-loop between S1 and S2 transmembrane domains (FIG. 1). Antibody reactivity was assayed by immunoblotting of different whole cell lysates LNCaP (FIGS. 4 and 5A) and PC-3M (FIG. 5B), i.e. TRPV6 positive). A band around 95-100 kDa with the expected size for the glycosylated/mature form of TRPV6 channel was observed in LNCaP cells for ab79a and ab79b and ab79c (FIG. 4A). An unspecific 50 kDa band was also detected with Ab79a-c (FIG. 4B). A multitude of mostly smaller size bands were observed with Ab79c (FIG. 4A); no reliable staining was observed with commercial anti-TRPV6 antibodies designed to recognize an intracellular epitope of human TRPV6 (Sigma SAB2106366, and Santa-Cruz sc-28763), (FIG. 4 A).

In conclusion, immunoblotting in denaturing conditions allowed to evaluate antibody specificity to the particular epitope against which it was raised. Ab79a was the better antibody able to detect a band of the expected size for the monomeric TRPV6 protein.

### Detection of TRPV6 Protein Expression Using Antibodiy Mab82.

One monoclonal antibody was raised against peptide situated at the N-terminus end of the p-loop (between S5 and S6) in the pore region (FIG. 1). Antibody reactivity was assayed by immunoblotting of different whole cell lysates LNCaP (FIG. 4B), i.e. TRPV6 positive). A band around 95-100 kDa with the expected size for the glycosylated/mature form of TRPV6 channel was observed in LNCaP cells only for mab82, no reliable staining was observed with commercial anti-TRPV6 antibodies designed to recognize an intracellular epitope of human TRPV6 (Sigma SAB2106366, and Santa-Cruz sc-28763).

In conclusion, immunoblotting in denaturing conditions allowed to evaluate antibody specificity to the particular epitope against which it was raised. mAb82 was the only antibody able to detect a band of the expected size for the monomeric TRPV6 protein (in comparison with commercial antibodies tested).

### Antibody 79a Validation Using Knockdown, (Over)-Expression, and Knockout Models

Four siRNAs were used in this study to carry out a specific knockdown of TRPV6 expression. The list of the siRNA sequences is indicated in Table I They target the first, the seventh, the eleventh, and the thirteenth exon of the mRNA. First, the quantitative real-time PCR of the TRPV6 channel was performed in the LNCaP cells transfected either with the 40 μM control siRNA (luciferase) or with the 40 μM siRNAs 1 to 4 against TRPV6 channel or their mixture as compared to HPRT gene expression. The knockdown at the level of the mRNA decay was more than 60% of efficiency which was reflected by the corresponding immunoblotting of the protein lysates from the siRNAs treated LNCaP cells and the quantification of the bands as compared to AKTB (data not shown).

As a next step an (over)-expression system was used. It should be noted that it is extremely difficult to have a cell system in vitro which does not express TRPV6 channel, since the presence of 2 mM of calcium in almost every medium makes the expression of TRPV6 advantageous for the cell survival. The data show an increase varying from slight to strong in the TRPV6 expression, suggesting that the ≈100 kDa band is specific.

### Flow Cytometry

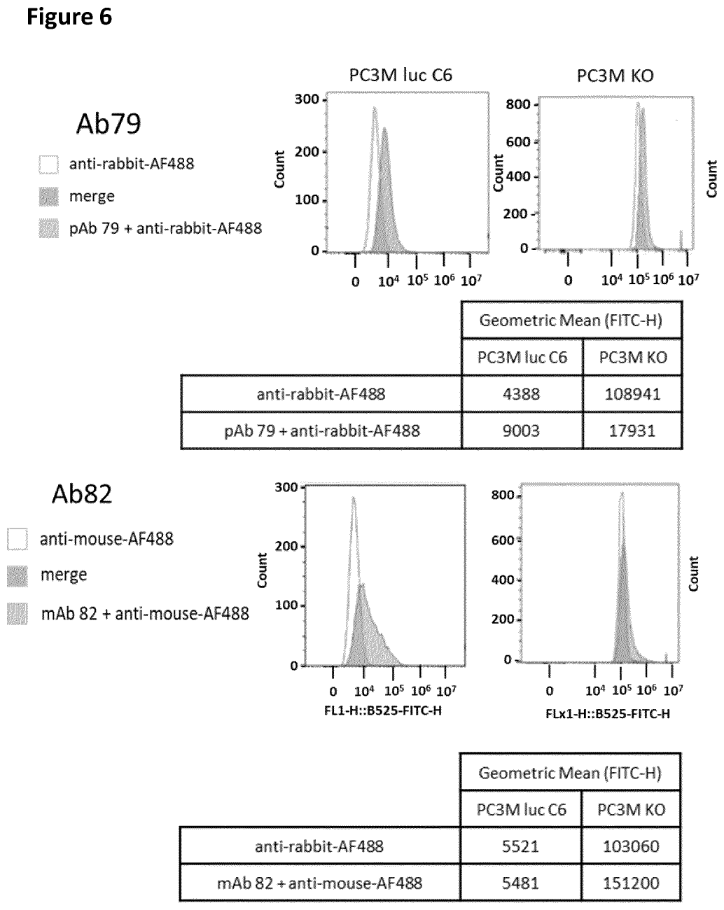

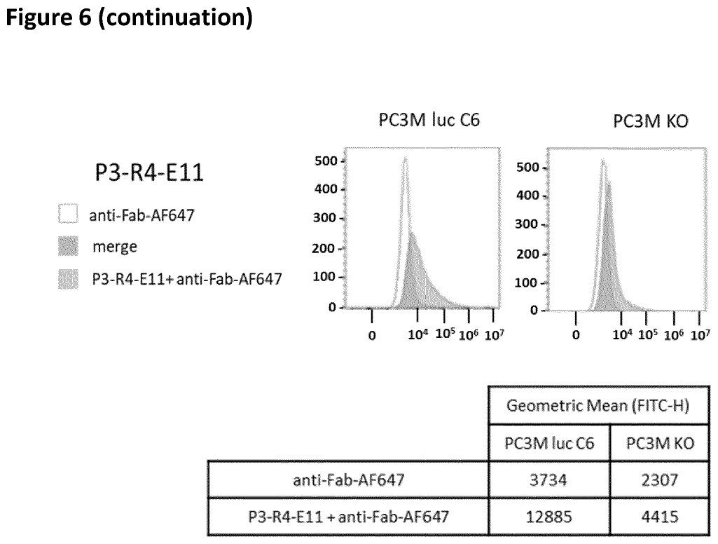

To test the specificity of antibody on TRPV6 PC3M expressing TRPV6 cell line transfected by luc C6 and PC3M KO cell line were test. Results showed a strong binding on PC3M luc C6 cells for Ac 79, 82 or P3-R4-E11 on PC3M luc C6 cells, whereas a weaker signal was observed on PC3M KO (FIG. 6).

### Example 2: Use of Anti-TRPV6 Antibodies Raised Against Extracellular Epitopes for the Diagnosis and Prognosis of Cancer in Clinical Samples

Rabbit polyclonal anti-TRPV6 antibody No:79a was used to perform immunohistochemistry (IHC) using human clinical samples from prostate resection specimens including normal prostate (bladder cancer resection specimen and adenocarcinomas with the Gleason score 7 (data not shown).

These data confirm the negative expression of the TRPV6 channel in the healthy prostate which corresponds to the published data before (Wissenbach et al 2001; 2004; Peng et al. 2001; Raphael et al. 2014). Therefore, the rabbit polyclonal anti-TRPV6 antibody No:79a can be used for diagnostic/prognostic purposes. Finally, IHC of the tumors slices derived from tumors grafted using HAP-1^{trpv6-/-} and HAP-1^{trpv6+/+} cell lines was carried out using rabbit polyclonal anti-TRPV6 antibody No:79a. The results show that rabbit polyclonal anti-TRPV6 antibody No:79a is not capable of recognizing any TRPV6 channel in HAP-1^{trpv-/-} formed tumors validating both knockout model and antibody specificity.

Mouse monoclonal mab82a antibody was used to perform immunohistochemistry (IHC) using human clinical samples from prostate resection specimens including normal prostate (bladder cancer resection specimen), and adenocarcinomas with the Gleason score 7.

These data confirm the negative expression of the TRPV6 channel in the healthy prostate which corresponds to the published data before (Wissenbach et al 2001; 2004; Peng et al. 2001; Raphaël et al. 2014). Therefore, the rabbit polyclonal anti-TRPV6 antibody mab82a can be used for diagnostic/prognostic purposes.

### Example 3: Treatment with Anti-TRPV6 Antibodies Raised Against Extracellular Epitopes Modulates TRPV6 Channel Activity

### Antibody Treatments Increase Store-Operated Capacitive Calcium Entry in PCa Cells

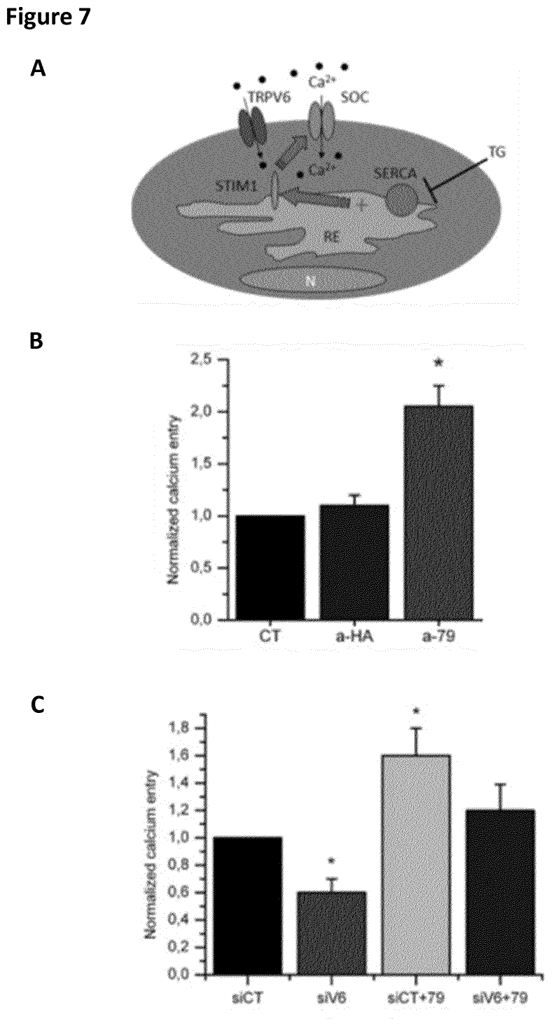

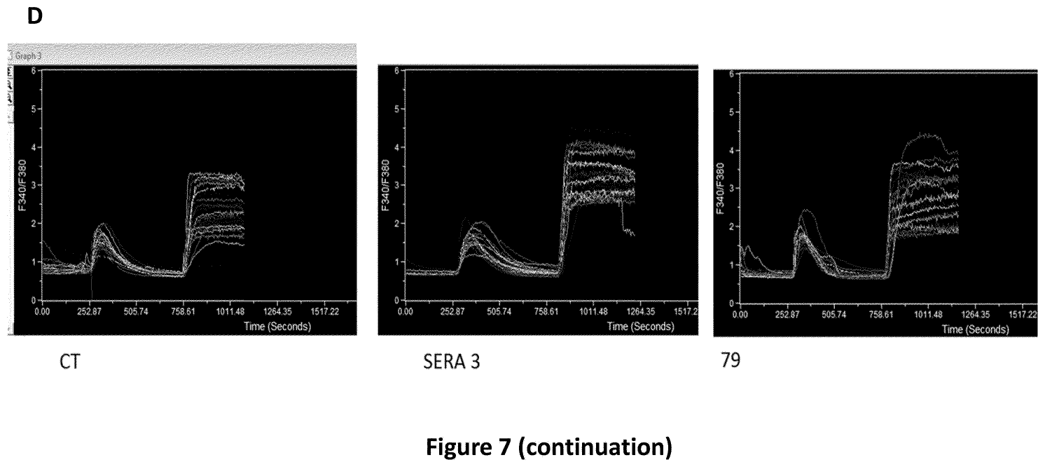

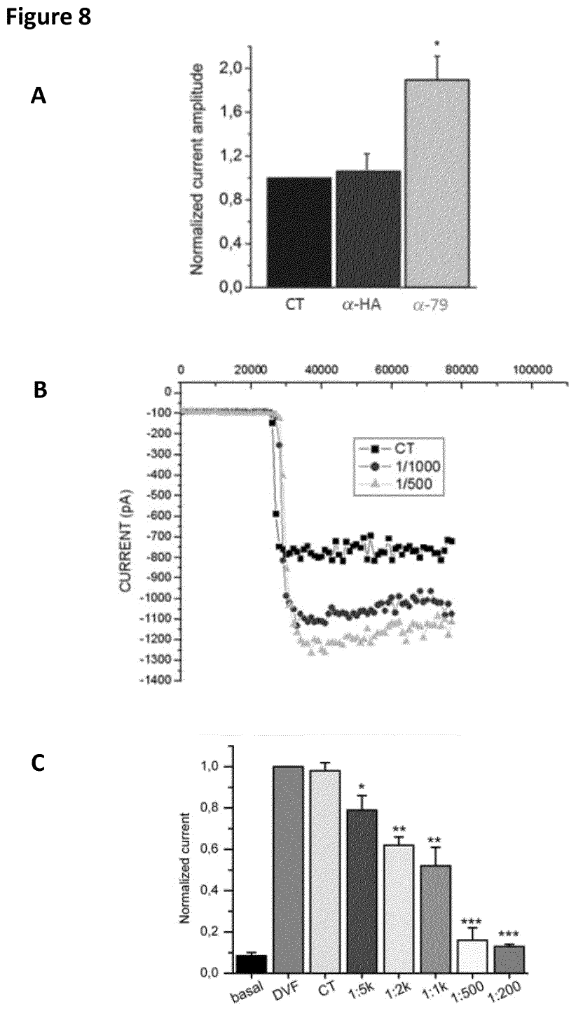

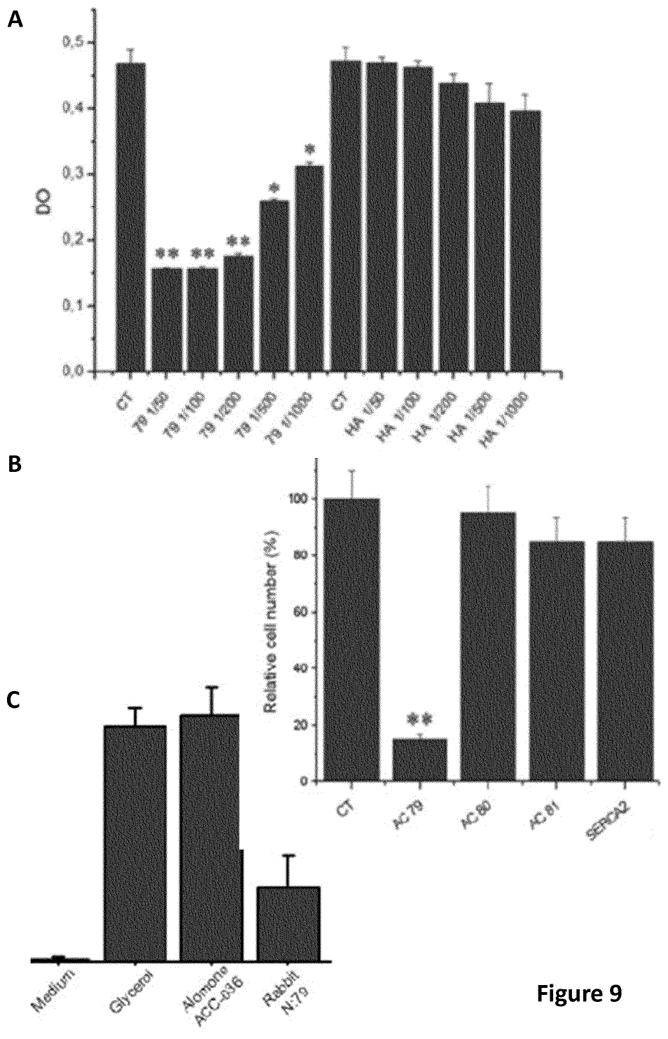

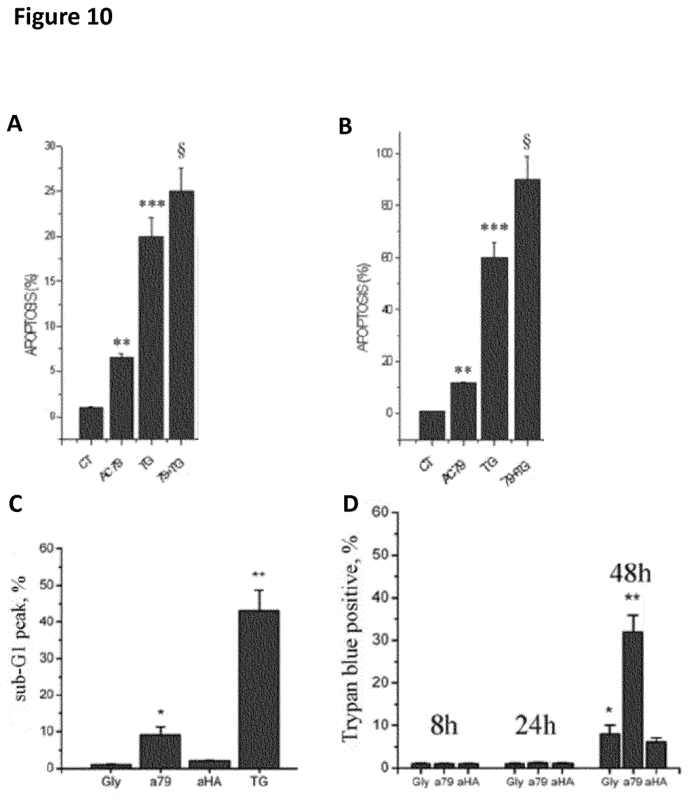

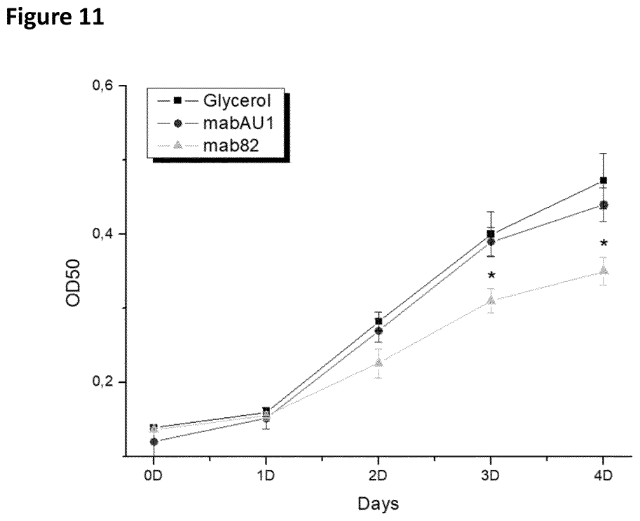

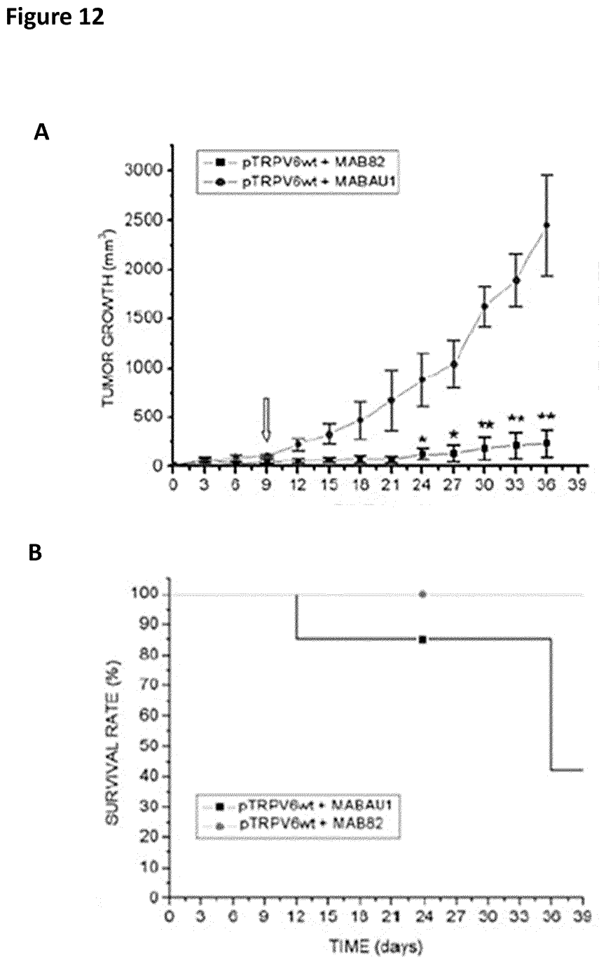

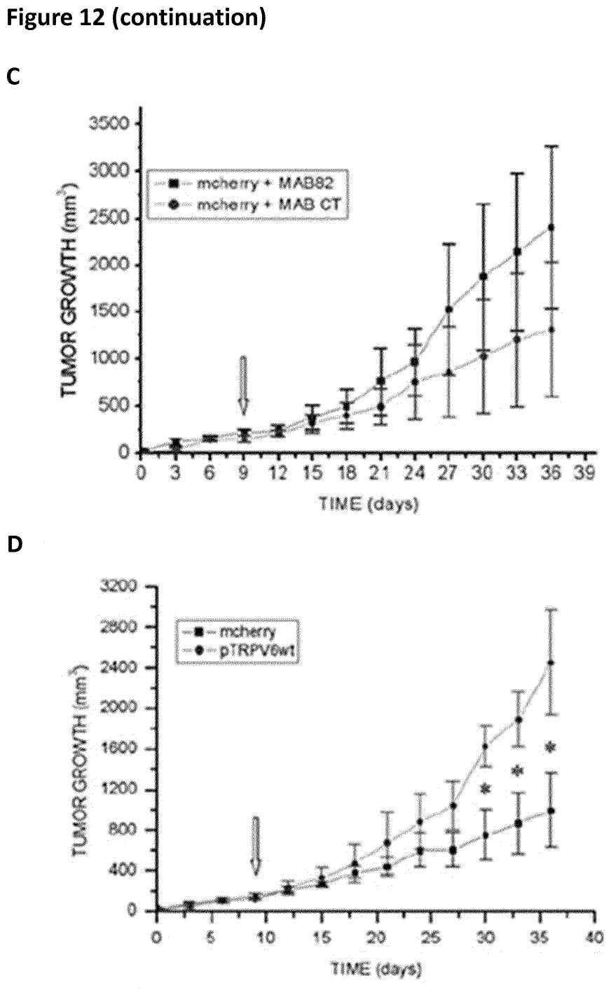

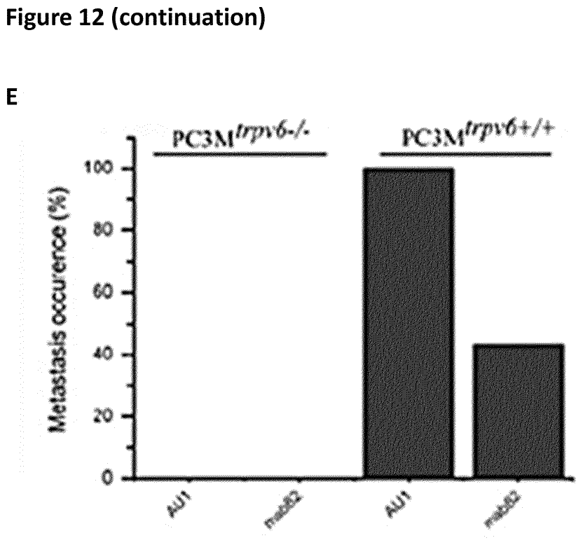

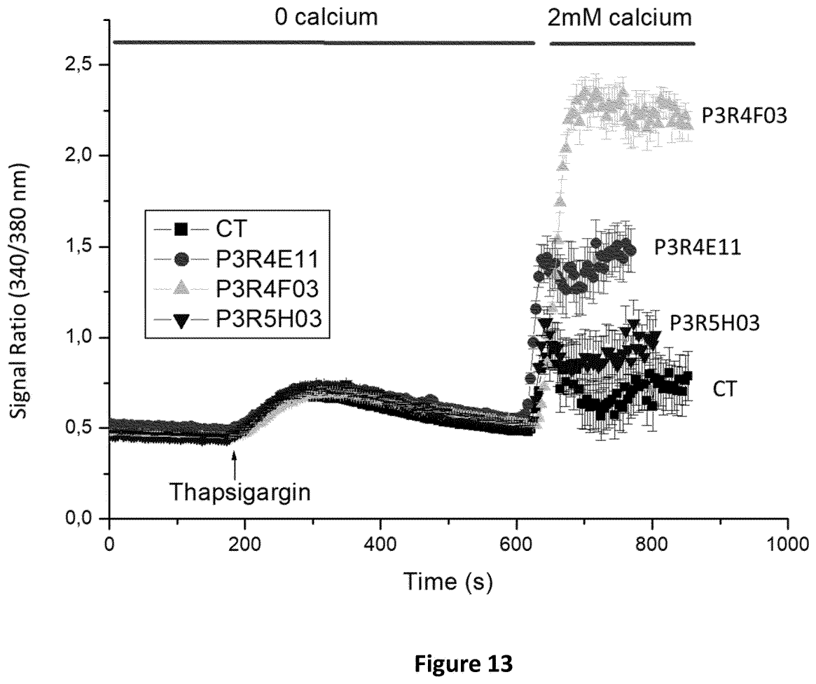

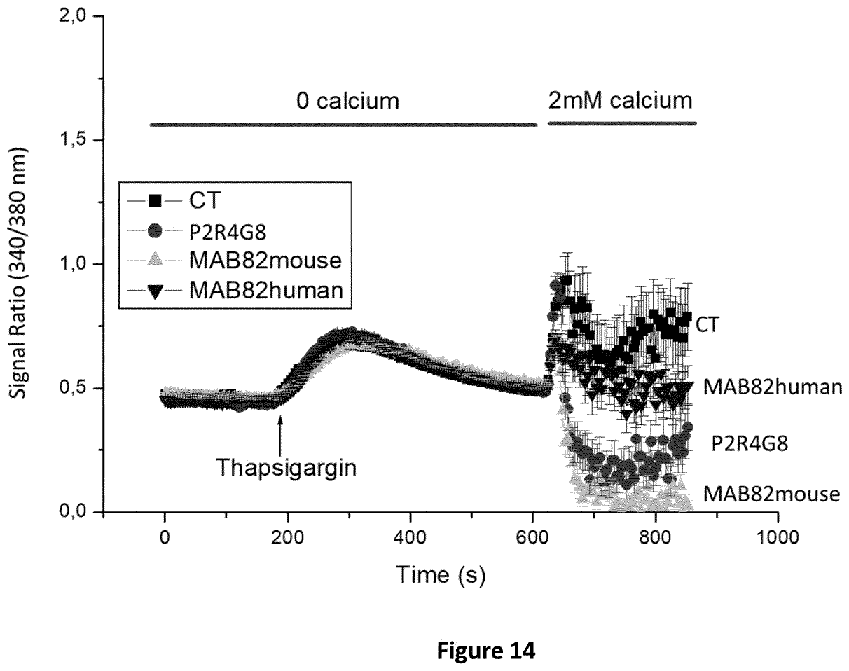

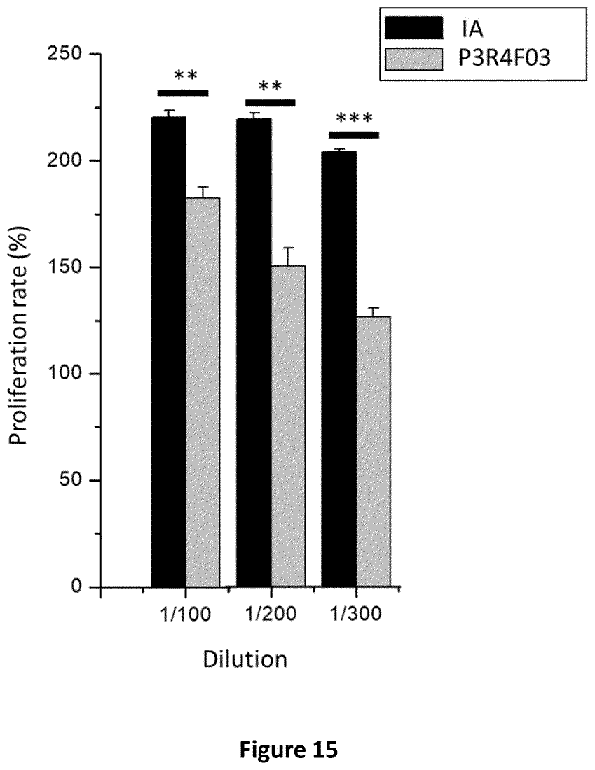

TRPV6 was shown as an important element of store-operated calcium entry (SOCE) into the PCa cells allowing the use of this mechanism to detect and analyze TRPV6 activity (Raphaël et al. 2014). This mechanism is triggered by the emptying of calcium stores in endoplasmic reticulum (ER). Inhibition of the SERCA pump with Thapsigargin (1 μM) is used to induce calcium leak which will empty calcium stores and thus activate store-operated (SOC) channels like Orail or TRPC1, which, in turn, will activate TRPV6 channel taking an important part, at least half, in the amplification of the calcium entry inside the cells (Raphaël et al. 2014; FIG. 7A).

In the experimental protocol cells are first incubated with the solution containing no calcium in order to create an outward gradient which is amplified by the use of 1 μM of Thapsigargin blocking a SERCA pump and thus not allowing calcium to be re-uptaken into ER. This artificial condition will create a great lack of calcium crucial for cell survival and thus will open a so-called store-operated channels (SOC). The addition of 2 mM of calcium will provide a calcium entry via SOCs, which, in turn, will activate TRPV6 channel taking an important part in the amplification of the calcium entry inside the cells (Raphaël et al. 2014). The preincubation of PCa cells like LNCaP for 5 min with either glycerol (CT), or rabbit polyclonal anti-HA or polyclonal anti-TRPV6 antibodies No. 79a, all normalized in 0.5 μg/μl at 1/500 dilutions, led to differential effects, such as a selective and significant increase in the SOCE levels in the case of polyclonal antibody No. 79a (FIG. 7B). To prove that these effects are mediated via TRRPV6 channel, antibody was subjected to the control while using siRNA strategy for TRPV6 knockdown. The SOCE was significantly decreased while knocking down TRPV6 channel and the antibody No. 79a-mediated increase in SOCE (siCT+No. 79a) was significantly attenuated as compared to siTRPV6+No. 79 treatment (FIG. 7C). Thus, both polyclonal antibody, Nos: 79 activate TRPV6 which amplifies SOCE and let enter greater amount of calcium inside cells.

Preincubation of cells with antibody 83 results in an increase in capacitive calcium input over control, showing that TRPV6-mediated calcium input is increased with antibody 83 similarly like antibody 79 (FIG. 7D).

### Polyclonal Antibodies 79a Affects Directly TRPV6-Induced Currents

A golden standard in extracellular antibodies action on the ion channel is a technique of electrophysiology allowing measurement of ion currents passing through the particular channel since each of them has a unique conducting feature or signature. The specificity of the polyclonal developed antibody No. 79 was verified by measuring their effect upon whole cell currents recorded from HEK cells transfected with vEFlap-5′UTR-TRPV6 wt_CMVp-mCherry (FIG. 8). Cells were initially bathed, as described, in a physiological solution containing 10 mM Ca²⁺, known to block TRPV6 activity (Singh et al., Sci Adv. 2018, 4, eaau6088; Derler et al., J Physiol. 2006, 577, 31-44; Niemeyer et al., Proc Natl AcadS ci USA. 2001, 98, 3600-5). TRPV6-specific currents were evoked by exchanging the extracellular (bath) solution to the divalent cation free (DVF) solution, commonly known to stimulate TRPV6 activity (Derler et al. 2006; Niemeyer et al. 2001). Rabbit polyclonal anti-TRPV6 antibody No. 79a was capable of significantly increasing the current while binding to TRPV6 channel (1/500, 0.5 μg/μl) as compared to the control antibody of the same isotype rabbit polyclonal anti-HA epitope antibody (FIG. 8A). To confirm the specificity of this binding, the dose-response experiment was conducted and showed the progressive activation of the TRPV6 channel (FIG. 8B).

### Monoclonal Antibodies 82 Affects Directly TRPV6-Induced Currents

A golden standard in extracellular antibodies action on the ion channel is a technique of electrophysiology allowing measurement of ion currents passing through the particular channel since each of them has a unique conducting feature or signature. The specificity of the monoclonal mab82 was verified by measuring their effect upon whole cell currents recorded from HEK cells transfected with vEFlap-5′UTR-TRPV6 wt_CMVp-mCherry (FIG. 8C). Cells were initially bathed, as described, in a physiological solution containing 10 mM Ca²⁺, known to block TRPV6 activity (Singh et al., Sci Adv. 2018, 4, eaau6088; Derler et al., J Physiol. 2006, 577, 31-44; Niemeyer et al., Proc Natl AcadS ci USA. 2001, 98, 3600-5). TRPV6-specific currents were evoked by exchanging the extracellular (bath) solution to the divalent cation free (DVF) solution, commonly known to stimulate TRPV6 activity (Derler et al. 2006; Niemeyer et al. 2001). Mouse monoclonal anti-TRPV6 antibody mab82a was capable of significantly decreasing the current while binding to TRPV6 channel as compared to the control (CT) (FIG. 8C). The specificity of the developed monoclonal antibody No. 82a (mab82) was verified by measuring its effect upon whole cell currents recorded from the HEK cell transfected with the vEFlap-5′UTR-TRPV6 wt_CMVp-mCherry vector and treated with the mouse monoclonal anti-TRPV6 antibody No. 82a (mab82). The mouse monoclonal anti-TRPV6 antibody No. 82a was applied at the series of increasing concentrations (1:5000, 1:2000, 1:1000 followed by 1:500 and 1:200 dilutions) in order to establish a dose-dependent effect of this antibody on the TRPV6 currents. FIG. 8C summarizes the observed TRPV6 currents by increasing concentrations of the applied antibody and suggests concentration-dependent effects.

Other Monoclonal Antibodies Against Epitope P2 Affect directly TRPV6-Induced Currents

Preincubation of cells with humanized mab82 or P2R4G08 results in a decrease in capacitive calcium input over control, showing that TRPV6-mediated calcium input is decreased with humanized mab82 or P2R4G08 similarly like murine mab82 (FIG. 14).

### Monoclonal Antibodies Against Epitope P3 Affect Directly TRPV6-Induced Currents

Preincubation of cells with P3R4F03, P3R4E11 or P3R5H03 results in an increase in capacitive calcium input over control, showing that TRPV6-mediated calcium input is increased with antibody P3R4F03, P3R4E11 or P3R5H03 (FIG. 13).

### Example 4: Treatment with Anti-TRPV6 Antibodies Raised Against Extracellular Epitopes Decreases Cell Survival Via Modulation of TRPV6 Activity

### Antibody 79

Once the direct action of the antibodies on the TRPV6 channel was proved, the next question was whether the rabbit polyclonal anti-TRPV6 antibody No. 79a is capable of influencing PCa cell survival in vitro. For that, LNCaP cells were incubated for 72 hours either with glycerol (CT) or different dilutions of polyclonal anti-TRPV6 antibody No. 79a or control antibody anti-HA and cell survival was measured by MTS assay (FIG. 9A). A strong reduction in cell survival was observed with polyclonal antibodies 79a whereas no effect was observed with control anti-HA antibody.

A panel of additional techniques was used since cell survival assay-which is based on cytochrome p-450 activity evaluation-is a complex assay measuring both cell proliferation and cell death.

Cell count assay with various control antibodies (rabbit polyclonal anti-SERCA2B; rabbit polyclonal antibodies No. 80 and 81 targeting intracellular epitopes of TRPV6 channel. Peptide 80 (hTRPV6 64-78; QRRESWAQSRDEQNL (SEQ ID NO: 189); peptide 81 hTRPV6 692-707; HTRGSEDLDKDSVEKL (SEQ ID NO: 190); rabbit polyclonal anti-GFP of the same isotype) confirmed the results of the survival assay and highlight the specificity of polyclonal antibody No. 79a (FIG. 9B).

Cell survival assay (MTS) of LNCaP cells treated either with medium, equivalent quantity of glycerol as control (CT), anti-TRPV6 antibody No. 79(79a) or commercial anti-TRPV6 antibody (Alomone #ACC-036) for 3 days showed the specificity of the effects due to anti-TRPV6 antibody No. 79(79a) (FIG. 9C).

Classical apoptosis assay by Hoechst staining was used to confirm the hypothesis of apoptosis induction by polyclonal anti-TRPV6 antibody No. 79a. Thapsigargin 1 μM for 3 days was used as a positive control since it induces calcium-dependent apoptosis in the long-term treatment. Quantification of apoptotic cells showed a significant death rate induced by 3 day treatment with polyclonal anti-TRPV6 antibody No. 79a for LNCaP (FIG. 10A), and HEK cells (FIG. 10B) which are much more apoptosis sensitive as compared to PCa cells. In addition, the presence of polyclonal antibody No. 79a at the same time as TG potentiates apoptosis significantly as compared to TG-only treatments, in LNCaP for antibody 79 (FIG. 10A) and HEK for 79a antibody (FIG. 10B).

Cell cycle assay showed a distinct subG1-peak in LNCaP cells treated with polyclonal antibody No. 79a suggesting that anti-TRPV6 antibody act via inducing apoptosis rather than decreasing proliferation (FIG. 10C). Finally, to exclude necrosis as a possible mechanism, a time series of 8, 24 and 48 hours was performed using trypan blue staining showing the late appearance of the stained cells, as an indicator of middle to late apoptosis where the membrane integrity is compromised (FIG. 10D).

### Antibody 82 (Ab 82)

Once the direct action of the antibodies on the TRPV6 channel was proved, the next question was whether the mouse monoclonal anti-TRPV6 antibody mab82, is capable of influencing cancer cell survival in vitro. For that, LNCaP cells were incubated for 24, 48, 72, 96 hours either with glycerol (CT) or anti-TRPV6 antibody mab82 or control antibody mabAU1 and cell survival was measured by CellTiter (FIG. 11). A strong reduction in cell survival was observed with mab82 whereas no effect was observed with control mabAU1 at 72 and 96 hours.

### Monoclonal Antibodies Against Epitope P3

Once the cells calcium modulation of the antibodies was proved, the next question was whether anti-TRPV6 antibody P3R4F03 is capable of influencing PCa cell survival in vitro. For that, LNCaP cells were incubated for 72 hours either with different dilutions of P3R4F03 antibody or irrelevant antibody (IA) and cell survival was measured by Cell titer glo assay (FIG. 15). A reduction in cell survival was observed with P3R4F03 antibody compare to irrelevant antibody (IA).

### Example 5: Treatment with Anti-TRPV6 Antibodies Raised Against Extracellular Epitopes Suppresses Tumor Growth and Metastasis Progression In Vivo

The therapeutic effect of monoclonal antibody 82 (mab82) was tested using an immunodeficient “swiss nude” mouse model grafted with 2×10E6 cells from stable clones of PC3M^{trPv6-/-}-pmCherry and PC3M^{trp-/-}—pTRPV6 wt cell lines as described previously for PCa cells (Raphaël et al., 2014). Antibody treatment was performed using 2 groups, PC3M cells with and without TRPV6 channel, and in each group 2 subgroups for the treatment, either control mouse monoclonal anti-AU1 antibody or anti-TRPV6 antibody mab82. For ease of in vivo monitoring, antibodies were previously coupled to Cf790 fluorophore. A kinetic study was performed with different doses (0.5 μg to 15 μg/mouse) determined based on closest publication (Bleeker et al., Br J Haematol., 2008, 140, 303-12). The EC₅₀ (minimal quantity needed to maintain maximum duration) was calculated and the dose of 100 μg/kg (3 μg per mouse) was chosen for both mab82 and control antibody anti-AU1 of the same isotype (IgG2a). The biodistribution of both antibodies was also studied immediately after bolus injection and their distribution in various organs of the body 30 min after antibody injection. As soon as tumors became visible, the treatments with either control anti-AU1 or experimental mab82 antibody, started twice per week, yielding convincing data, such as follows: tumors in the PC3M^{trpv6-/-}-pTRPV6 wt group mab82-treated subgroup decreased in size leaving only a blue slightly visible border or nothing 3 weeks after the beginning of treatment as compared to control anti-AU1-treated subgroup. After 5 weeks of treatment with mab82 this blue border traces disappeared leaving some connective tissue-like traces or nothing. Overall, the treatments being started at day 9 (arrow, FIG. 12A) gave already significant difference in size beginning from day 24 after grafting and 15 from the treatment, making the difference striking at the end of experiment. It should be noted that the tumors in the PC3M^{trpv-/-} -pTRPV6 wt group anti-AU1-treated subgroup were extremely aggressive (because of the TRPV6 channel) reflecting the general survival rate (FIG. 12B), while PC3M^{trpv6-/-} -pmCherry group whatever the subgroup (antibody treatment) was, did not show any significant difference between subgroups (FIG. 12C). As to the difference between PC3M^{trpv6-/-}—pmCherry and PC3M^{trpv6-/-} —pTRPV6 wt groups in tumor growth, the latter group has shown significant difference in tumor growth because of the TRPV6 channel present (FIG. 12D). mCherry imaging in vivo was done weekly using small animal imaging system and demonstrated tumor suppression. As soon as tumors reached their maximal size, they were excised and mice left alive and imaging in vivo was continued weekly during at least three months.

Metastasis study clearly indicated that there was no metastasis in the PC3M^{trpv6-/-} -pmCherry group, while there was 100% metastatic potential in the PC3M^{trpv6-/-}—pTRPV6 wt group treated with the control AU1 antibody, and only 40% in PC3M^{trp-/-} —pTRPV6 wt group treated with mab82 (FIG. 12E). In this group 70% of metastasis were due to the presence of aggressive tumors in the neck region, tumors usually extra-vascularized and difficult to excise due to excessive expansion.

In conclusion, mab82 treatment proved to be a prospective therapeutic solution to solid tumors growth and metastasis occurrence in vivo.

Brief description of additional amino acid sequences useful for practicing the invention.

| SEQ ID NO: 1: Human TRPV6 protein (UniProtKB/Swiss-Prot: NP_061116.5 or | |
|---|---|
| Q9H1D0.3) | |
| 1 | MGPLQGDGGP ALGGADVAPR LSPVRVWPRP QAPKEPALHP MGLSLPKEKG LILCLWSKFC |
| 61 | RWFQRRESWA QSRDEQNLLQ QKRIWESPLL LAAKDNDVQA LNKLLKYEDC KVHQRGAMGE |
| 121 | TALHIAALYD NLEAAMVLME AAPELVFEPM TSELYEGQTA LHIAVVNQNM NLVRALLARR |
| 181 | ASVSARATGT AFRRSPCNLI YFGEHPLSFA ACVNSEEIVR LLIEHGADIR AQDSLGNTVL |
| 241 | HILILQPNKT FACQMYNLLL SYDRHGDHLQ PLDLVPNHQG LTPFKLAGVE GNTVMFQHLM |
| 301 | QKRKHTQWTY GPLTSTLYDL TEIDSSGDEQ SLLELIITTK KREARQILDQ TPVKELVSLK |
| 361 | WKRYGRPYFC MLGAIYLLYI ICFTMCCIYR PLKPRINNRT SPRDNTLLQQ KLLQEAYMTP |
| 421 | KDDIRLVGEL VTVIGAIIIL LVEVPDIFRM GVTRFFGQTI LGGPFHVLII TYAFMVLVTM |
| 481 | VMRLISASGE VVPMSFALVL GWCNVMYFAR GFQMLGPFTI MIQKMIFGDL MRFCWLMAVV |
| 541 | ILGFASAFYI IFQTEDPEEL GHFYDYPMAL FSTFELELTI IDGPANYNVD LPFMYSITYA |
| 601 | AFAIIATLLM LNLLIAMMGD THWRVAHERD ELWRAQIVAT TVMLERKLPR CLWPRSGICG |
| 661 | REYGLGDRWF LRVEDRQDLN RQRIQRYAQA FHTRGSEDLD KDSVEKLELG CPFSPHLSLP |
| 721 | MPSVSRSTSR SSANWERLRQ GTLRRDLRGI INRGLEDGES WEYQI |

TABLE I
Sequences of the antibodies of the invention

| | | |
|---|---|---|
| | Description | |
| SEQ ID | of the | |
| NO: | sequence | Sequence |

Monoclonal antibody 83 (mAb83)

| | | |
|---|---|---|
| 17 | VL-CDR1 | QSLLDSDGRTY |
| | VL-CDR2 | LVS |
| 18 | VL-CDR3 | WQGTHFPQT |
| 19 | VL-FR1 | DVVMTQTPLTLSVTIGQPASISCKSS |
| 20 | VL-FR2 | LNWLLQRPGQSPKRLIY |
| 21 | VL-FR3 | KLDSGVPDRFTGSGSGTDFTLKISRVEAEDLGVYYC |
| 22 | VL-FR4 | FGGGTKLDIK |
| 23 | VL | DVVMTQTPLTLSVTIGQPASISCKSSQSLLDSDGRTYLNWLLQRPG |
| | | QSPKRLIYLVSKLDSGVPDRFTGSGSGTDFTLKISRVEAEDLGVYY |
| | | CWQGTHFPQTFGGGTKLDIK |
| 24 | VL-CL | DVVMTQTPLTLSVTIGQPASISCKSSQSLLDSDGRTYLNWLLQR |
| | | PGQSPKRLIYLVSKLDSGVPDRFTGSGSGTDFTLKISRVEAEDL |
| | | GVYYCWQGTHFPQTFGGGTKLDIKRADAAPTVSIFPPSSEQLTS |
| | | GGASVVCFLNNFYPKDINVKWKIDGSERQNGVLNSWTDQDSKDS |
| | | TYSSSTLTLTKDEYERHNSYTCEATHKTSTSPIVKSFNRNEC |
| 25 | VL* | GATGTTGTGATGACCCAGACTCCTCTCACTTTGTCGGTTACCATTG |
| | | GACAACCAGCCTCCATCTCTTGCAAGTCAAGTCAGAGCCTCTTAGA |
| | | TAGTGATGGAAGGACATATTTGAATTGGTTGTTACAGAGGCCAGGC |
| | | CAGTCTCCAAAGCGCCTAATCTATCTGGTGTCTAAACTGGACTCTG |
| | | GAGTCCCTGACAGATTCACTGGCAGTGGATCAGGGACAGATTTCAC |
| | | ACTGAAAATCAGCAGAGTGGAGGCTGAGGATTTGGGAGTTTATTAT |
| | | TGCTGGCAAGGTACACATTTTCCTCAGACGTTCGGTGGAGGCACCA |
| | | AGCTGGACATCAAA |
| 26 | VL-CL* | GATGTTGTGATGACCCAGACTCCTCTCACTTTGTCGGTTACCATTG |
| | | GACAACCAGCCTCCATCTCTTGCAAGTCAAGTCAGAGCCTCTTAGA |
| | | TAGTGATGGAAGGACATATTTGAATTGGTTGTTACAGAGGCCAGGC |
| | | CAGTCTCCAAAGCGCCTAATCTATCTGGTGTCTAAACTGGACTCTG |
| | | GAGTCCCTGACAGATTCACTGGCAGTGGATCAGGGACAGATTTCAC |
| | | ACTGAAAATCAGCAGAGTGGAGGCTGAGGATTTGGGAGTTTATTAT |
| | | TGCTGGCAAGGTACACATTTTCCTCAGACGTTCGGTGGAGGCACCA |
| | | AGCTGGACATCAAACGGGCTGATGCTGCACCAACTGTATCCATCTT |
| | | CCCACCATCCAGTGAGCAGTTAACATCTGGAGGTGCCTCAGTCGTG |
| | | TGCTTCTTGAACAACTTCTACCCCAAAGACATCAATGTCAAGTGGA |
| | | AGATTGATGGCAGTGAACGACAAAATGGCGTCCTGAACAGTTGGAC |
| | | TGATCAGGACAGCAAAGACAGCACCTACAGCATGAGCAGCACCCTC |
| | | ACGTTGACCAAGGACGAGTATGAACGACATAACAGCTATACCTGTG |
| | | AGGCCACTCACAAGACATCAACTTCACCCATTGTCAAGAGCTTCAA |
| | | CAGGAATGAGTGT |
| 27 | VH-CDR1 | GFDFSRYW |
| 28 | VH-CDR2 | INPYSSTI |
| 29 | VH-CDR3 | AGKDFFAY |
| 30 | VH-FR1 | EVKLIESGGGLVQPGGSLKLSCAAS |
| 31 | VH-FR2 | MSWVRQAPGKGLEWIGE |
| 32 | VH-FR3 | NYTPSLKDKFIISRDNAKNTLYLQMRKVRSEDTALYYC |
| 33 | VH-FR4 | WGQGTLVTVSA |
| 34 | VH | EVKLIESGGGLVQPGGSLKLSCAASGFDFSRYWMSWVRQAPGKGLE |
| | | WIGEINPYSSTINYTPSLKDKFIISRDNAKNTLYLQMRKVRSEDTA |
| | | LYYCAGKDFFAYWGQGTLVTVSA |
| 35 | VH-CH | EVKLIESGGGLVQPGGSLKLSCAASGFDFSRYWMSWVRQAPGKGLE |
| | | WIGEINPYSSTINYTPSLKDKFIISRDNAKNTLYLQMRKVRSEDTA |
| | | LYYCAGKDFFAYWGQGTLVTVSAAKTTPPSVYPLAPGSAAQTNSMV |
| | | TLGCLVKGYFPEPVTVTWNSGSLSSGVHTFPAVLQSDLYTLSSSVT |
| | | VPSSTWPSETVTCNVAHPASSTKVDKKIVPRDCGCKPCICTVPEVS |
| | | SVFIFPPKPKDVLTITLTPKVTCVVVDISKDDPEVQFSWFVDDVEV |
| | | HTAQTQPREEQFNSTERSVSELPIMHQDWLNGKEFKCRVNSAAFPA |
| | | PIEKTISKTKGRPKAPQVYTIPPPKEQMAKDKVSLTCMITDFFPED |
| | | ITVEWQWNGQPAENYKNTQPIMDTDGSYFVYSKLNVQKSNWEAGNT |
| | | FTCSVLHEGLHNHHTEKSLSHSPGK |
| 36 | VH* | GAGGTGAAGCTTATCGAGTCTGGAGGTGGCCTGGTGCAGCCTGGAG |
| | | GATCCCTGAAACTCTCCTGTGCAGCCTCAGGATTCGATTTTAGTAG |
| | | ATACTGGATGAGTTGGGTCCGGCAGGCTCCAGGGAAAGGGCTAGAA |
| | | TGGATTGGAGAAATTAATCCATATAGCAGTACGATAAACTATACGC |
| | | CATCTCTAAAGGATAAATTCATCATCTCCAGAGACAACGCCAAAAA |
| | | TACGCTGTACCTGCAAATGAGGAAAGTGAGATCTGAGGACACAGCC |
| | | CTTTATTACTGTGCCGGGAAGGATTTTTTTGCTTACTGGGGCCAAG |
| | | GGACTCTGGTCACTGTCTCTGCA |
| 37 | VH-CH* | GAGGTGAAGCTTATCGAGTCTGGAGGTGGCCTGGTGCAGCCTGGAG |
| | | GATCCCTGAAACTCTCCTGTGCAGCCTCAGGATTCGATTTTAGTAG |
| | | ATACTGGATGAGTTGGGTCCGGCAGGCTCCAGGGAAAGGGCTAGAA |
| | | TGGATTGGAGAAATTAATCCATATAGCAGTACGATAAACTATACGC |
| | | CATCTCTAAAGGATAAATTCATCATCTCCAGAGACAACGCCAAAAA |
| | | TACGCTGTACCTGCAAATGAGGAAAGTGAGATCTGAGGACACAGCC |
| | | CTTTATTACTGTGCCGGGAAGGATTTTTTTGCTTACTGGGGCCAAG |
| | | GGACTCTGGTCACTGTCTCTGCAGCCAAAACGACACCCCCATCTGT |
| | | CTATCCACTGGCCCCTGGATCTGCTGCCCAAACTAACTCCATGGTG |
| | | ACCCTGGGATGCCTGGTCAAGGGCTATTTCCCTGAGCCAGTGACAG |
| | | TGACCTGGAACTCTGGATCCCTGTCCAGCGGTGTGCACACCTTCCC |
| | | AGCTGTCCTGCAGTCTGACCTCTACACTCTGAGCAGCTCAGTGACT |
| | | GTCCCCTCCAGCACCTGGCCCAGCGAGACCGTCACCTGCAACGTTG |
| | | CCCACCCGGCCAGCAGCACCAAGGTGGACAAGAAAATTGTGCCCAG |
| | | GGATTGTGGTTGTAAGCCTTGCATATGTACAGTCCCAGAAGTATCA |
| | | TCTGTCTTCATCTTCCCCCCAAAGCCCAAGGATGTGCTCACCATTA |
| | | CTCTGACTCCTAAGGTCACGTGTGTTGTGGTAGACATCAGCAAGGA |
| | | TGATCCCGAGGTCCAGTTCAGCTGGTTTGTAGATGATGTGGAGGTG |
| | | CACACAGCTCAGACGCAACCCCGGGAGGAGCAGTTCAACAGCACTT |
| | | TCCGCTCAGTCAGTGAACTTCCCATCATGCACCAGGACTGGCTCAA |
| | | TGGCAAGGAGTTCAAATGCAGGGTCAACAGTGCAGCTTTCCCTGCC |
| | | CCCATCGAGAAAACCATCTCCAAAACCAAAGGCAGACCGAAGGCTC |
| | | CACAGGTGTACACCATTCCACCTCCCAAGGAGCAGATGGCCAAGGA |
| | | TAAAGTCAGTCTGACCTGCATGATAACAGACTTCTTCCCTGAAGAC |
| | | ATTACTGTGGAGTGGCAGTGGAATGGGCAGCCAGCGGAGAACTACA |
| | | AGAACACTCAGCCCATCATGGACACAGATGGCTCTTACTTCGTCTA |
| | | CAGCAAGCTCAATGTGCAGAAGAGCAACTGGGAGGCAGGAAATACT |
| | | TTCACCTGCTCTGTGTTACATGAGGGCCTGCACAACCACCATACTG |
| | | AGAAGAGCCTCTCCCACTCTCCTGGTAAA |

Monoclonal antibody 82 (mAb82)
Mouse monoclonal

| | | |
|---|---|---|
| 38 | VL-CDR1 | QSLLYSSNQKNY |
| | VL-CDR2 | WAS |
| 39 | VL-CDR3 | QQYYRYPT |
| 40 | VL-FR1 | DIVMSQSPSSLAVSVGEKVTMSCKSS |
| 41 | VL-FR2 | LAWYQQKPGQSPKLLIY |
| 42 | VL-FR3 | TRESGVPDRFTGSGSGTDFTLTISSVKAEDLAVYYC |
| 43 | VL-FR4 | FGGGTKLEIK |
| 44 | VL-FR4) | FGGGTKLAVL |
| 45 | VL | DIVMSQSPSSLAVSVGEKVTMSCKSSQSLLYSSNQKNYLAWYQQKP |
| | | GQSPKLLIYWASTRESGVPDRFTGSGSGTDFTLTISSVKAEDLAVY |
| | | YCQQYYRYPTFGGGTKLEIK |
| 46 | VL | DIVMSQSPSSLAVSVGEKVTMSCKSSQSLLYSSNQKNYLAWYQQKP |
| | | GQSPKLLIYWASTRESGVPDRFTGSGSGTDFTLTISSVKAEDLAVY |
| | | YCQQYYRYPTFGGGTKLAVL |
| 47 | VL-CL | DIVMSQSPSSLAVSVGEKVTMSCKSSQSLLYSSNQKNYLAWYQQKP |
| | | GQSPKLLIYWASTRESGVPDRFTGSGSGTDFTLTISSVKAEDLAVY |
| | | YCQQYYRYPTFGGGTKLEIKRADAAPTVSIFPPSSEQLTSGGASVV |
| | | CFLNNFYPKDINVKWKIDGSERQNGVLNSWTDQDSKDSTYSMSSTL |
| | | TLTKDEYERHNSYTCEATHKTSTSPIVKSFNRNEC |
| 48 | VL-CL* | GACATTGTGATGTCACAGTCTCCATCCTCCCTAGCTGTGTCAGTTG |
| | | GAGAGAAGGTTACTATGAGCTGCAAGTCCAGTCAGAGCCTTTTATA |
| | | TAGTAGCAATCAAAAGAACTACTTGGCCTGGTACCAGCAGAAACCA |
| | | GGGCAGTCTCCTAAACTGCTGATTTACTGGGCATCCACTAGGGAAT |
| | | CTGGGGTCCCTGATCGCTTCACAGGCAGTGGATCTGGGACAGATTT |
| | | CACTCTCACCATCAGCAGTGTGAAGGCTGAAGACCTGGCAGTTTAT |
| | | TACTGTCAGCAATATTATAGGTATCCGACGTTCGGTGGAGGCACCA |
| | | AGCTGGAAATCAAACGGGCTGATGCTGCACCAACTGTATCCATCTT |
| | | CCCACCATCCAGTGAGCAGTTAACATCTGGAGGTGCCTCAGTCGTG |
| | | TGCTTCTTGAACAACTTTTACCCCAAAGACATCAATGTCAAGTGGA |
| | | AGATTGATGGCAGTGAACGACAAAATGGCGTCCTGAACAGTTGGAC |
| | | TGATCAGGACAGCAAAGACAGCACCTACAGCATGAGCAGCACCCTC |
| | | ACGTTGACCAAGGACGAGTATGAACGACATAACAGCTATACCTGTG |
| | | AGGCCACTCACAAGACATCAACTTCACCCATTGTCAAGAGCTTCAA |
| | | CAGGAATGAGTGT |
| 49 | VH-CDR1 | GFDFRRYW |
| 50 | VH-CDR2 | INPDSSTI |
| 51 | VH-CDR3 | ARSASSHYFDY |
| 52 | VH-FR1 | EVKLLESGGGLVQPGGSLKLSCAAS |
| 53 | VH-FR2 | MSWVRQAPGKGLEWIGE |
| 54 | VH-FR3 | NYTPSLKDKFIISRDNAKNTLYLQMSKVRSEDTALYYC |
| 55 | VH-FR4 | WGQGTTLTVSS |
| 56 | VH | EVKLLESGGGLVQPGGSLKLSCAASGFDFRRYWMSWVRQAPGKGLE |
| | | WIGEINPDSSTINYTPSLKDKFIISRDNAKNTLYLQMSKVRSEDTA |
| | | LYYCARSASSHYFDYWGQGTTLTVSS |
| 57 | VH-CH | EVKLLESGGGLVQPGGSLKLSCAASGFDFRRYWMSWVRQAPGKGLE |
| | | WIGEINPDSSTINYTPSLKDKFIISRDNAKNTLYLQMSKVRSEDTA |
| | | LYYCARSASSHYFDYWGQGTTLTVSSAKTTAPSVYPLAPVCGDTTG |
| | | SSVTLGCLVKGYFPEPVTLTWNSGSLSSGVHTFPAVLQSDLYTLSS |
| | | SVTVTSSTWPSQSITCNVAHPASSTKVDKKIEPRGPTIKPCPPCKC |
| | | PAPNLLGGPSVFIFPPKIKDVLMISLSPIVTCVVVDVSEDDPDVQI |
| | | SWFVNNVEVHTAQTQTHREDYNSTLRVVSALPIQHQDWMSGKEFKC |
| | | KVNNKDLPAPIERTISKPKGSVRAPQVYVLPPPEEEMTKKQVTLTC |
| | | MVTDEMPEDIYVEWTNNGKTELNYKNTEPVLDSDGSYFMYSKLRVE |
| | | KKNWVERNSYSCSVVHEGLHNHHTTKSFSRTPGK |
| 58 | VH-CH* | GAGGTGAAGCTTCTCGAGTCTGGAGGTGGCCTGGTGCAGCCTGGAG |
| | | GATCCCTGAAACTCTCCTGTGCAGCCTCAGGATTCGATTTTAGAAG |
| | | ATACTGGATGAGTTGGGTCCGGCAGGCTCCAGGGAAAGGGCTAGAA |
| | | TGGATTGGAGAAATTAATCCAGATAGCAGTACGATAAACTATACGC |
| | | CATCTCTAAAGGATAAATTCATCATCTCCAGAGACAACGCCAAAAA |
| | | TACGCTGTACCTGCAAATGAGCAAAGTGAGATCTGAGGACACAGCC |
| | | CTTTATTACTGTGCAAGATCGGCTTCATCCCACTACTTTGACTACT |
| | | GGGGCCAAGGCACCACTCTCACAGTCTCCTCAGCCAAAACAACAGC |
| | | CCCATCGGTCTATCCACTGGCCCCTGTGTGTGGAGATACAACTGGC |
| | | TCCTCGGTGACTCTAGGATGCCTGGTCAAGGGTTATTTCCCTGAGC |
| | | CAGTGACCTTGACCTGGAACTCTGGATCCCTGTCCAGTGGTGTGCA |
| | | CACCTTCCCAGCTGTCCTGCAGTCTGACCTCTACACCCTCAGCAGC |
| | | TCAGTGACTGTAACCTCGAGCACCTGGCCCAGCCAGTCCATCACCT |
| | | GCAATGTGGCCCACCCGGCAAGCAGCACCAAGGTGGACAAGAAAAT |
| | | TGAGCCCAGAGGGCCCACAATCAAGCCCTGTCCTCCATGCAAATGC |
| | | CCAGCACCTAACCTCTTGGGTGGACCATCCGTCTTCATCTTCCCTC |
| | | CAAAGATCAAGGATGTACTCATGATCTCCCTGAGCCCCATAGTCAC |
| | | ATGTGTGGTGGTGGATGTGAGCGAGGATGACCCAGATGTCCAGATC |
| | | AGCTGGTTTGTGAACAACGTGGAAGTACACACAGCTCAGACACAAA |
| | | CCCATAGAGAGGATTACAACAGTACTCTCCGGGTGGTCAGTGCCCT |
| | | CCCCATCCAGCACCAGGACTGGATGAGTGGCAAGGAGTTCAAATGC |
| | | AAGGTCAACAACAAAGACCTCCCAGCGCCCATCGAGAGAACCATCT |
| | | CAAAACCCAAAGGGTCAGTAAGAGCTCCACAGGTATATGTCTTGCC |
| | | TCCACCAGAAGAAGAGATGACTAAGAAACAGGTCACTCTGACCTGC |
| | | ATGGTCACAGACTTCATGCCTGAAGACATTTACGTGGAGTGGACCA |
| | | ACAACGGGAAAACAGAGCTAAACTACAAGAACACTGAACCAGTCCT |
| | | GGACTCTGATGGTTCTTACTTCATGTACAGCAAGCTGAGAGTGGAA |
| | | AAGAAGAACTGGGTGGAAAGAAATAGCTACTCCTGTTCAGTGGTCC |
| | | ACGAGGGTCTGCACAATCACCACACGACTAAAAGCTTCTCCCGGAC |
| | | TCCGGGTAAA |

Humanized monoclonal

| | | |
|---|---|---|
| 59 | VL-CDR1 | QSLLYSSNQKNY |
| | VL-CDR2 | WAS |
| 60 | VL-CDR3 | QQYYRYPT |
| 61 | VL-FR1 | DIVMSQSPSSLAVSVGEKVTMSCKSS |
| 62 | VL-FR2 | LAWYQQKPGQSPKLLIY |
| 63 | VL-FR3 | TRESGVPDRFTGSGSGTDFTLTISSVKAEDLAVYYC |
| 64 | VL-FR4 | FGGGTKLEIK |
| 65 | VL-FR4 | FGGGTKLAVL |
| 66 | VL | DIVMSQSPSSLAVSVGEKVTMSCKSSQSLLYSSNQKNYLAWYQQKP |
| | | GQSPKLLIYWASTRESGVPDRFTGSGSGTDFTLTISSVKAEDLAVY |
| | | YCQQYYRYPTFGGGTKLEIK |
| 67 | VL | DIVMSQSPSSLAVSVGEKVTMSCKSSQSLLYSSNQKNYLAWYQQKP |
| | | GQSPKLLIYWASTRESGVPDRFTGSGSGTDFTLTISSVKAEDLAVY |
| | | YCQQYYRYPTFGGGTKLAVL |
| 68 | VL-CL | DIVMSQSPSSLAVSVGEKVTMSCKSSQSLLYSSNQKNYLAWYQQKPG |
| | IgG1&IgG4 | QSPKLLIYWASTRESGVPDRFTGSGSGTDFTLTISSVKAEDLAVYYC |
| | | QQYYRYPTFGGGTKLEIKRTVAAPSVFIFPPSDEQLKSGTASVVCLL |
| | | NNFYPREAKVQWKVDNALQSGNSQESVTEQDSKDSTYSLSSTLTLSK |
| | | ADYEKHKVYACEVTHQGLSSPVTKSFNRGEC |
| 69 | VL-CL | GATATCGTTATGACTCAAAGTCCAGACTCGCTGGCAGTGAGCCTTG |
| | IgG1&IgG4* | GGGAGAGAGCCACTATAAATTGCAAGTCCAGTCAATCACTGCTGTA |
| | | TTCAAGCAACCAGAAAAACTATTTGGCGTGGTATCAGCAGAAGCCC |
| | | GGTCAGCCGCCTAAACTCCTAATTTACTGGGCTTCTACCCGCGAAT |
| | | CTGGCGTGCCTGACCGGTTTTCTGGATCCGGCTCCGGGACCGACTT |
| | | CACACTCACAATCAGCTCCTTACAGGCCGAAGATGTAGCTGTCTAT |
| | | TACTGTCAGCAGTACTACAGGTACCCCACGTTCGGTGGAGGCACCA |
| | | AGGTGGAGATTAAGCGAACGGTGGCTGCACCATCTGTCTTCATCTT |
| | | CCCGCCATCTGATGAGCAGTTGAAATCTGGAACTGCCTCTGTTGTG |
| | | TGCCTGCTGAATAACTTCTATCCCAGAGAGGCCAAAGTACAGTGGA |
| | | AGGTGGATAACGCCCTCCAATCGGGTAACTCCCAGGAGAGTGTCAC |
| | | AGAGCAGGACAGCAAGGACAGCACCTACAGCCTCAGCAGCACCCTG |
| | | ACGCTGAGCAAAGCAGACTACGAGAAACACAAAGTCTACGCCTGCG |
| | | AAGTCACCCATCAGGGCCTGAGCTCGCCCGTCACAAAGAGCTTCAA |
| | | CAGGGGAGAGTGTTAG |
| 70 | VH-CDR1 | GFDFRRYW |
| 71 | VH-CDR2 | INPDSSTI |
| 72 | VH-CDR3 | ARSASSHYFDY |
| 73 | VH-FR1 | EVKLLESGGGLVQPGGSLKLSCAAS |
| 74 | VH-FR2 | MSWVRQAPGKGLEWIGE |
| 75 | VH-FR3 | NYTPSLKDKFIISRDNAKNTLYLQMSKVRSEDTALYYC |
| 76 | VH-FR4 | WGQGTTLTVSS |
| 77 | VH | EVKLLESGGGLVQPGGSLKLSCAASGFDFRRYWMSWVRQAPGKGLE |
| | | WIGEINPDSSTINYTPSLKDKFIISRDNAKNTLYLQMSKVRSEDTA |
| | | LYYCARSASSHYFDYWGQGTTLTVSS |
| 78 | VH-CH | EVKLLESGGGLVQPGGSLKLSCAASGFDFRRYWMSWVRQAPGKGLEW |
| | (IgG1) | IGEINPDSSTINYTPSLKDKFIISRDNAKNTLYLQMSKVRSEDTALY |
| | | YCARSASSHYFDYWGQGTTLTVSSASTKGPSVFPLAPSSKSTSGGTA |
| | | ALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNV |
| | | FSCSVMHEALHNHYTQKSLSLSPGK |
| 79 | VH-CH | GAGGTTCAATTAGTAGAATCGGGAGGTGGTCTAGTTCAGCCGGGGG |
| | (IgG1* | GCTCCCTCCGTTTGTCATGTGCGGCTTCAGGATTCGACTTCCGGAG |
| | | GTATTGGATGCACTGGGTCCGGCAGGCACCCGGCAAAGGGCTTGTC |
| | | TGGGTGTCTAGGATCAACCCTGACAGCTCCACTATTAACTACACTC |
| | | CAAGTCTGAAAGACAAGTTTATAATCAGTCGCGATAATGCCAAGAA |
| | | TACCCTCTACCTGCAGATGTCTAAGGTGAGATCCGAGGATACCGCC |
| | | CTGTATTACTGCGCTCGATCTGCATCCAGCCATTATTTTGATTACT |
| | | GGGGCCAGGGAACACTGGTGACAGTGAGCAGCGCAAGCACCAAGGG |
| | | CCCATCGGTCTTCCCCCTGGCACCCTCCTCCAAGAGCACCTCTGGG |
| | | GGCACAGCGGCCCTGGGCTGCCTGGTCAAGGACTACTTCCCCGAAC |
| | | CGGTGACGGTGTCGTGGAACTCAGGCGCCCTGACCAGCGGCGTGCA |
| | | CACCTTCCCGGCTGTCCTACAGTCCTCAGGACTCTACTCCCTCAGC |
| | | AGCGTGGTGACCGTGCCCTCCAGCAGCTTGGGCACCCAGACCTACA |
| | | TCTGCAACGTGAATCACAAGCCCAGCAACACCAAGGTGGACAAGAA |
| | | AGTTGAGCCCAAATCTTGTGACAAAACTCACACATGCCCACCGTGC |
| | | CCAGCACCTGAACTCCTGGGGGGACCGTCAGTCTTCCTCTTCCCCC |
| | | CAAAACCCAAGGACACCCTCATGATCTCCCGGACCCCTGAGGTCAC |
| | | ATGCGTGGTGGTGGACGTGAGCCACGAAGACCCTGAGGTCAAGTTC |
| | | AACTGGTACGTGGACGGCGTGGAGGTGCATAATGCCAAGACAAAGC |
| | | CGCGGGAGGAGCAGTACAACAGCACGTACCGTGTGGTCAGCGTCCT |
| | | CACCGTCCTGCACCAGGACTGGCTGAATGGCAAGGAGTACAAGTGC |
| | | AAGGTCTCCAACAAAGCCCTCCCAGCCCCCATCGAGAAAACCATCT |
| | | CCAAAGCCAAAGGGCAGCCCCGAGAACCACAGGTGTACACCCTGCC |
| | | CCCATCCCGGGAGGAGATGACCAAGAACCAGGTCAGCCTGACCTGC |
| | | CTGGTCAAAGGCTTCTATCCCAGCGACATCGCCGTGGAGTGGGAGA |
| | | GCAATGGGCAGCCGGAGAACAACTACAAGACCACGCCTCCCGTGCT |
| | | GGACTCCGACGGCTCCTTCTTCCTCTACAGCAAGCTCACCGTGGAC |
| | | AAGAGCAGGTGGCAGCAGGGGAACGTCTTCTCATGCTCCGTGATGC |
| | | ATGAGGCTCTGCACAACCACTACACGCAGAAGAGCCTCTCCCTGTC |
| | | TCCGGGTAAATGA |
| 80 | VH-CH | EVKLLESGGGLVQPGGSLKLSCAASGFDFRRYWMSWVRQAPGKGLEW |
| | (IgG4) | IGEINPDSSTINYTPSLKDKFIISRDNAKNTLYLQMSKVRSEDTALY |
| | | YCARSASSHYFDYWGQGTTLTVSSASTKGPSVFPLAPCSRSTSESTA |
| | | ALGCLVKDYFPEPVTVSWNSGALTSGVHTFPAVLQSSGLYSLSSVVT |
| | | VPSSSLGTKTYTCNVDHKPSNTKVDKRVESKYGPPCPSCPAPEELGG |
| | | PSVFLFPPKPKDTLMISRTPEVTCVVVDVSQEDPEVQFNWYVDGVEV |
| | | HNAKTKPREEQFNSTYRVVSVLTVLHQDWLNGKEYKCKVSNKGLPSS |
| | | IEKTISKAKGQPREPQVYTLPPSQEEMTKNQVSLTCLVKGFYPSDIA |
| | | VEWESNGQPENNYKTTPPVLDSDGSFFLYSRLTVDKSRWQEGNVFSC |
| | | SVMHEALHNHYTQKSLSLSLGK |
| 81 | VH-CH | GAGGTTCAATTAGTAGAATCGGGAGGTGGTCTAGTTCAGCCGGGGG |
| | (IgG4)* | GCTCCCTCCGTTTGTCATGTGCGGCTTCAGGATTCGACTTCCGGAG |
| | | GTATTGGATGCACTGGGTCCGGCAGGCACCCGGCAAAGGGCTTGTC |
| | | TGGGTGTCTAGGATCAACCCTGACAGCTCCACTATTAACTACACTC |
| | | CAAGTCTGAAAGACAAGTTTATAATCAGTCGCGATAATGCCAAGAA |
| | | TACCCTCTACCTGCAGATGTCTAAGGTGAGATCCGAGGATACCGCC |
| | | CTGTATTACTGCGCTCGATCTGCATCCAGCCATTATTTTGATTACT |
| | | GGGGCCAGGGAACACTGGTGACAGTGAGCAGCGCAAGCACCAAGGG |
| | | CCCATCGGTCTTCCCCCTGGCGCCCTGCTCCAGGAGCACCTCCGAG |
| | | AGCACAGCCGCCCTGGGCTGCCTGGTCAAGGACTACTTCCCCGAAC |
| | | CGGTGACGGTGTCGTGGAACTCAGGCGCCCTGACCAGCGGCGTGCA |
| | | CACCTTCCCGGCTGTCCTACAGTCCTCAGGACTCTACTCCCTCAGC |
| | | AGCGTGGTGACCGTGCCCTCCAGCAGCTTGGGCACGAAGACCTACA |
| | | CCTGCAACGTAGATCACAAGCCCAGCAACACCAAGGTGGACAAGAG |
| | | AGTTGAGTCCAAATATGGTCCCCCATGCCCATCATGCCCAGCACCT |
| | | GAGTTCCTGGGGGGACCATCAGTCTTCCTGTTCCCCCCAAAACCCA |
| | | AGGACACTCTCATGATCTCCCGGACCCCTGAGGTCACGTGCGTGGT |
| | | GGTGGACGTGAGCCAGGAAGACCCCGAGGTCCAGTTCAACTGGTAC |
| | | GTGGATGGCGTGGAGGTGCATAATGCCAAGACAAAGCCGCGGGAGG |
| | | AGCAGTTCAACAGCACGTACCGTGTGGTCAGCGTCCTCACCGTCCT |
| | | GCACCAGGACTGGCTGAACGGCAAGGAGTACAAGTGCAAGGTCTCC |
| | | AACAAAGGCCTCCCGTCCTCCATCGAGAAAACCATCTCCAAAGCCA |
| | | AAGGGCAGCCCCGAGAGCCACAGGTGTACACCCTGCCCCCATCCCA |
| | | GGAGGAGATGACCAAGAACCAGGTCAGCCTGACCTGCCTGGTCAAA |
| | | GGCTTCTACCCCAGCGACATCGCCGTGGAGTGGGAGAGCAATGGGC |
| | | AGCCGGAGAACAACTACAAGACCACGCCTCCCGTGCTGGACTCCGA |
| | | CGGCTCCTTCTTCCTCTACAGCAGGCTAACCGTGGACAAGAGCAGG |
| | | TGGCAGGAGGGGAATGTCTTCTCATGCTCCGTGATGCATGAGGCTC |
| | | TGCACAACCACTACACACAGAAGAGCCTCTCCCTGTCTCTGGGTAA |
| | | ATAA |

Chimeric monoclonal

| | | |
|---|---|---|
| 82 | VL-CL | DIVMTQSPDSLAVSLGERATINCKSSQSLLYSSNQKNYLAWYQQKPG |
| | (IgG1 &IgG4) | QPPKLLIYWASTRESGVPDRFSGSGSGTDFTLTISSLQAEDVAVYYC |
| | | QQYYRYPTFGGGTKVEIKRTVAAPSVFIFPPSDEQLKSGTASVVCLL |
| | | NNFYPREAKVQWKVDNALQSGNSQESVTEQDSKDSTYSLSSTLTLSK |
| | | ADYEKHKVYACEVTHQGLSSPVTKSFNRGEC |
| 83 | VL-CL | GACATTGTGATGTCACAGTCTCCATCCTCCCTAGCTGTGTCAGTTG |
| | (IgG1& | GAGAGAAGGTTACTATGAGCTGCAAGTCCAGTCAGAGCCTTTTATA |
| | IgG4)* | TAGTAGCAATCAAAAGAACTACTTGGCCTGGTACCAGCAGAAACCA |
| | | GGGCAGTCTCCTAAACTGCTGATTTACTGGGCATCCACTAGGGAAT |
| | | CTGGGGTCCCTGATCGCTTCACAGGCAGTGGATCTGGGACAGATTT |
| | | CACTCTCACCATCAGCAGTGTGAAGGCTGAAGACCTGGCAGTTTAT |
| | | TACTGTCAGCAATATTATAGGTATCCGACGTTCGGTGGAGGCACCA |
| | | AGCTGGAAATCAAACGAACGGTGGCTGCACCATCTGTCTTCATCTT |
| | | CCCGCCATCTGATGAGCAGTTGAAATCTGGAACTGCCTCTGTTGTG |
| | | TGCCTGCTGAATAACTTCTATCCCAGAGAGGCCAAAGTACAGTGGA |
| | | AGGTGGATAACGCCCTCCAATCGGGTAACTCCCAGGAGAGTGTCAC |
| | | AGAGCAGGACAGCAAGGACAGCACCTACAGCCTCAGCAGCACCCTG |
| | | ACGCTGAGCAAAGCAGACTACGAGAAACACAAAGTCTACGCCTGCG |
| | | AAGTCACCCATCAGGGCCTGAGCTCGCCCGTCACAAAGAGCTTCAA |
| | | CAGGGGAGAGTGTTAG |
| 84 | VH-CH | EVQLVESGGGLVQPGGSLRLSCAASGFDFRRYWMHWVRQAPGKGLVW |
| | (IgG1) | VSRINPDSSTINYTPSLKDKFIISRDNAKNTLYLQMSKVRSEDTALY |
| | | YCARSASSHYFDYWGQGTLVTVSSASTKGPSVFPLAPSSKSTSGGTA |
| | | ALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNV |
| | | FSCSVMHEALHNHYTQKSLSLSPGK |
| 85 | VH-CH | GAGGTGAAGCTTCTCGAGTCTGGAGGTGGCCTGGTGCAGCCTGGAG |
| | (IgG1)* | GATCCCTGAAACTCTCCTGTGCAGCCTCAGGATTCGATTTTAGAAG |
| | | ATACTGGATGAGTTGGGTCCGGCAGGCTCCAGGGAAAGGGCTAGAA |
| | | TGGATTGGAGAAATTAATCCAGATAGCAGTACGATAAACTATACGC |
| | | CATCTCTAAAGGATAAATTCATCATCTCCAGAGACAACGCCAAAAA |
| | | TACGCTGTACCTGCAAATGAGCAAAGTGAGATCTGAGGACACAGCC |
| | | CTTTATTACTGTGCAAGATCGGCTTCATCCCACTACTTTGACTACT |
| | | GGGGCCAAGGCACCACTCTCACAGTCTCCTCAGCAAGCACCAAGGG |
| | | CCCATCGGTCTTCCCCCTGGCACCCTCCTCCAAGAGCACCTCTGGG |
| | | GGCACAGCGGCCCTGGGCTGCCTGGTCAAGGACTACTTCCCCGAAC |
| | | CGGTGACGGTGTCGTGGAACTCAGGCGCCCTGACCAGCGGCGTGCA |
| | | CACCTTCCCGGCTGTCCTACAGTCCTCAGGACTCTACTCCCTCAGC |
| | | AGCGTGGTGACCGTGCCCTCCAGCAGCTTGGGCACCCAGACCTACA |
| | | TCTGCAACGTGAATCACAAGCCCAGCAACACCAAGGTGGACAAGAA |
| | | AGTTGAGCCCAAATCTTGTGACAAAACTCACACATGCCCACCGTGC |
| | | CCAGCACCTGAACTCCTGGGGGGACCGTCAGTCTTCCTCTTCCCCC |
| | | CAAAACCCAAGGACACCCTCATGATCTCCCGGACCCCTGAGGTCAC |
| | | ATGCGTGGTGGTGGACGTGAGCCACGAAGACCCTGAGGTCAAGTTC |
| | | AACTGGTACGTGGACGGCGTGGAGGTGCATAATGCCAAGACAAAGC |
| | | CGCGGGAGGAGCAGTACAACAGCACGTACCGTGTGGTCAGCGTCCT |
| | | CACCGTCCTGCACCAGGACTGGCTGAATGGCAAGGAGTACAAGTGC |
| | | AAGGTCTCCAACAAAGCCCTCCCAGCCCCCATCGAGAAAACCATCT |
| | | CCAAAGCCAAAGGGCAGCCCCGAGAACCACAGGTGTACACCCTGCC |
| | | CCCATCCCGGGAGGAGATGACCAAGAACCAGGTCAGCCTGACCTGC |
| | | CTGGTCAAAGGCTTCTATCCCAGCGACATCGCCGTGGAGTGGGAGA |
| | | GCAATGGGCAGCCGGAGAACAACTACAAGACCACGCCTCCCGTGCT |
| | | GGACTCCGACGGCTCCTTCTTCCTCTACAGCAAGCTCACCGTGGAC |
| | | AAGAGCAGGTGGCAGCAGGGGAACGTCTTCTCATGCTCCGTGATGC |
| | | ATGAGGCTCTGCACAACCACTACACGCAGAAGAGCCTCTCCCTGTC |
| | | TCCGGGTAAATGA |
| 86 | VH-CH | EVQLVESGGGLVQPGGSLRLSCAASGFDFRRYWMHWVRQAPGKGLVW |
| | (IgG4) | VSRINPDSSTINYTPS |
| | | LKDKFIISRDNAKNTLYLQMSKVRSEDTALYYCARSASSHYFDYWGQ |
| | | GTLVTVSSASTKGPSVFPLAPCSRSTSESTAALGCLVKDYFPEPVTV |
| | | SWNSGALTSGVHTFPAVLQSSGLYSLSSVVTVPSSSLGTKTYTCNVD |
| | | HKPSNTKVDKRVESKYGPPCPSCPAPEFLGGPSVFLFPPKPKDTLMI |
| | | SRTPEVTCVVVDVSQEDPEVQFNWYVDGVEVHNAKTKPREEQENSTY |
| | | RVVSVLTVLHQDWLNGKEYKCKVSNKGLPSSIEKTISKAKGQPREPQ |
| | | VYTLPPSQEEMTKNQVSLTCLVKGFYPSDIAVEWESNGQPENNYKTT |
| | | PPVLDSDGSFFLYSRLTVDKSRWQEGNVESCSVMHEALHNHYTQKSL |
| | | SLSLGK |
| 87 | VH-CH | GAGGTGAAGCTTCTCGAGTCTGGAGGTGGCCTGGTGCAGCCTGGAG |
| | (IgG4)* | GATCCCTGAAACTCTCCTGTGCAGCCTCAGGATTCGATTTTAGAAG |
| | | ATACTGGATGAGTTGGGTCCGGCAGGCTCCAGGGAAAGGGCTAGAA |
| | | TGGATTGGAGAAATTAATCCAGATAGCAGTACGATAAACTATACGC |
| | | CATCTCTAAAGGATAAATTCATCATCTCCAGAGACAACGCCAAAAA |
| | | TACGCTGTACCTGCAAATGAGCAAAGTGAGATCTGAGGACACAGCC |
| | | CTTTATTACTGTGCAAGATCGGCTTCATCCCACTACTTTGACTACT |
| | | GGGGCCAAGGCACCACTCTCACAGTCTCCTCAGCAAGCACCAAGGG |
| | | CCCATCGGTCTTCCCCCTGGCGCCCTGCTCCAGGAGCACCTCCGAG |
| | | AGCACAGCCGCCCTGGGCTGCCTGGTCAAGGACTACTTCCCCGAAC |
| | | CGGTGACGGTGTCGTGGAACTCAGGCGCCCTGACCAGCGGCGTGCA |
| | | CACCTTCCCGGCTGTCCTACAGTCCTCAGGACTCTACTCCCTCAGC |
| | | AGCGTGGTGACCGTGCCCTCCAGCAGCTTGGGCACGAAGACCTACA |
| | | CCTGCAACGTAGATCACAAGCCCAGCAACACCAAGGTGGACAAGAG |
| | | AGTTGAGTCCAAATATGGTCCCCCATGCCCATCATGCCCAGCACCT |
| | | GAGTTCCTGGGGGGACCATCAGTCTTCCTGTTCCCCCCAAAACCCA |
| | | AGGACACTCTCATGATCTCCCGGACCCCTGAGGTCACGTGCGTGGT |
| | | GGTGGACGTGAGCCAGGAAGACCCCGAGGTCCAGTTCAACTGGTAC |
| | | GTGGATGGCGTGGAGGTGCATAATGCCAAGACAAAGCCGCGGGAGG |
| | | AGCAGTTCAACAGCACGTACCGTGTGGTCAGCGTCCTCACCGTCCT |
| | | GCACCAGGACTGGCTGAACGGCAAGGAGTACAAGTGCAAGGTCTCC |
| | | AACAAAGGCCTCCCGTCCTCCATCGAGAAAACCATCTCCAAAGCCA |
| | | AAGGGCAGCCCCGAGAGCCACAGGTGTACACCCTGCCCCCATCCCA |
| | | GGAGGAGATGACCAAGAACCAGGTCAGCCTGACCTGCCTGGTCAAA |
| | | GGCTTCTACCCCAGCGACATCGCCGTGGAGTGGGAGAGCAATGGGC |
| | | AGCCGGAGAACAACTACAAGACCACGCCTCCCGTGCTGGACTCCGA |
| | | CGGCTCCTTCTTCCTCTACAGCAGGCTAACCGTGGACAAGAGCAGG |
| | | TGGCAGGAGGGGAATGTCTTCTCATGCTCCGTGATGCATGAGGCTC |
| | | TGCACAACCACTACACACAGAAGAGCCTCTCCCTGTCTCTGGGTAA |
| | | ATAA |

Monoclonal antibody P2-R4-G08

| | | |
|---|---|---|
| 88 | VL-CDR1 | SSDVGGYGQ |
| | VL-CDR2 | SDS |
| 89 | VL-CDR3 | SSYTSYSTRV |
| 90 | VL-FR1 | QSVLTQPASVSGSPGQSITISCAGT |
| 91 | VL-FR2 | VSWYQQHPGKAPKLMIY |
| 92 | VL-FR3 | SRPSGVSNRFSGSKSGNTASLTISGLQAEDEADYYC |
| 93 | VL-FR4 | FGGGTKLEIK |
| 94 | VL-FR4 | FGGGTKLAVL |
| 95 | VL | QSVLTQPASVSGSPGQSITISCAGTSSDVGGYGQVSWYQQHPGKAP |
| | | KLMIYSDSSRPSGVSNRFSGSKSGNTASLTISGLQAEDEADYYCSS |
| | | YTSYSTRVFGGGTKLEIK |
| 96 | VL | QSVLTQPASVSGSPGQSITISCAGTSSDVGGYGQVSWYQQHPGKAP |
| | | KLMIYSDSSRPSGVSNRFSGSKSGNTASLTISGLQAEDEADYYCSS |
| | | YTSYSTRVFGGGTKLAVL |
| 97 | VL-CL | QSVLTQPASVSGSPGQSITISCAGTSSDVGGYGQVSWYQQHPGKAP |
| | | KLMIYSDSSRPSGVSNRFSGSKSGNTASLTISGLQAEDEADYYCSS |
| | | YTSYSTRVFGGGTKLEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC |
| 98 | VH-CDR1 | GFTFSNYG |
| 99 | VH-CDR2 | ISGSSRSI |
| 100 | VH-CDR3 | VRSSYYYGMDV |
| 101 | VH-FR1 | EVQLVESGGSLVKPGGSLRLSCAAS |
| 102 | VH-FR2 | MNWVRQAPGKGLEWISG |
| 103 | VH-FR3 | GYADFVKGRFTISRDNAKNSLYLQMNSLRAEDTAVYYC |
| 104 | VH-FR4 | WGRGTLVTVSS |
| 105 | VH | EVQLVESGGSLVKPGGSLRLSCAASGFTFSNYGMNWVRQAPGKGLE |
| | | WISGISGSSRSIGYADFVKGRFTISRDNAKNSLYLQMNSLRAEDTA |
| | | VYYCVRSSYYYGMDVWGRGTLVTVSS |
| 106 | VH-CH | EVQLVESGGSLVKPGGSLRLSCAASGFTFSNYGMNWVRQAPGKGLE |
| | | WISGISGSSRSIGYADFVKGRFTISRDNAKNSLYLQMNSLRAEDTA |
| | | VYYCVRSSYYYGMDVWGRGTLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKC |
| | | KVSNKALPAPIEKTISKAKGQPREPQVYTLPPSRDELTKNQVSLTC |
| | | LVKGFYPSDIAVEWESNGQPENNYKTTPPVLDSDGSFFLYSKLTVD |
| | | KSRWQQGNVFSCSVMHEALHNHYTQKSLSLSPGK |
| 107 | VH-CH | ASTKGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKKVEPKS |
| | | CDKTHTCPPCPAPEAAGGPSVFLFPPKPKDTLMISRTPEVTCVVVDVSHED |
| | | PEVKFNWYVDGVEVHNAKTKPREEQYNSTYRVVSVLTVLHQDWLNGKEYKC |
| | | KVSNKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQGNVF |
| | | SCSVMHEALHNHYTQKSLSLSPGK |

Monoclonal antibody P3-R4-E11

| | | |
|---|---|---|
| 108 | VL-CDR1 | SSDVGGYGY |
| | VL-CDR2 | YDS |
| 109 | VL-CDR3 | SSYTSQSTRV |
| 110 | VL-FR1 | QSVLTQPASVSGSPGQSITISCAGT |
| 111 | VL-FR2 | VSWYQQHPGKAPKLMIY |
| 112 | VL-FR3 | YRPSGVSNRFSGSKSGNTASLTISGLQAEDEADYYC |
| 113 | VL-FR4 | FGGGTKLEIK |
| 114 | VL-FR4 | FGGGTKLAVL |
| 115 | VL | QSVLTQPASVSGSPGQSITISCAGTSSDVGGYGYVSWYQQHPGKAPK |
| | | LMIYYDSYRPSGVSNRFSGSKSGNTASLTISGLQAEDEADYYCSSYTS |
| | | QSTRVEGGGTKLEIK |
| 116 | VL | QSVLTQPASVSGSPGQSITISCAGTSSDVGGYGYVSWYQQHPGKAPK |
| | | LMIYYDSYRPSGVSNRFSGSKSGNTASLTISGLQAEDEADYYCSSYTS |
| | | QSTRVFGGGTKLAVL |
| 117 | VL-CL | QSVLTQPASVSGSPGQSITISCAGTSSDVGGYGYVSWYQQHPGKAPK |
| | | LMIYYDSYRPSGVSNRFSGSKSGNTASLTISGLQAEDEADYYCSSYTS |
| | | QSTRVFGGGTKLEIKRTVAAPSVFIFPPSDEQLKSGTASVVCLLNNF |
| | | YPREAKVQWKVDNALQSGNSQESVTEQDSKDSTYSLSSTLTLSKAD |
| | | YEKHKVYACEVTHQGLSSPVTKSFNRGEC |
| 118 | VH-CDR1 | GFTFSNSG |
| 119 | VH-CDR2 | ISGSSRYI |
| 120 | VH-CDR3 | VRSNYGGMDV |
| 121 | VH-FR1 | EVQLVESGGSLVKPGGSLRLSCAAS |
| 122 | VH-FR2 | MNWVRQAPGKGLEWISG |
| 123 | VH-FR3 | GYADFVKGRFTISRDNAKNSLYLQMNSLRAEDTAVYYC |
| 124 | VH-FR4 | WGRGTLVTVSS |
| 125 | VH | EVQLVESGGSLVKPGGSLRLSCAASGFTFSNSGMNWVRQAPGKGLEWI |
| | | SGISGSSRYIGYADFVKGRFTISRDNAKNSLYLQMNSLRAEDTAVYY |
| | | CVRSNYGGMDVWGRGTLVTVSS |
| 126 | VH-CH | EVQLVESGGSLVKPGGSLRLSCAASGFTFSNSGMNWVRQAPGKGLEWI |
| | | SGISGSSRYIGYADFVKGRFTISRDNAKNSLYLQMNSLRAEDTAVYY |
| | | CVRSNYGGMDVWGRGTLVTVSSASTKGPSVFPLAPSSKSTSGGTAAL |
| | | GCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQ |
| | | QGNVFSCSVMHEALHNHYTQKSLSLSPGK |
| 127 | VH-CH | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |

Monoclonal antibody P3-R5-E06

| | | |
|---|---|---|
| 128 | VL-CDR1 | SSDVGGNYY |
| | VL-CDR2 | QDS |
| 129 | VL-CDR3 | SSYTGYSTRV |
| 130 | VL-FR1 | QSVLTQPASVSGSPGQSITISCAGT |
| 131 | VL-FR2 | VSWYQQHPGKAPKLMIY |
| 132 | VL-FR3 | NRPSGVSNRFSGSKSGNTASLTISGLQAEDEADYYC |
| 133 | VL-FR4 | FGGGTKLEIK |
| 134 | VL-FR4 | FGGGTKLAVL |
| 135 | VL | QSVLTQPASVSGSPGQSITISCAGTSSDVGGNYYVSWYQQHPGKAP |
| | | KLMIYQDSNRPSGVSNRFSGSKSGNTASLTISGLQAEDEADYYCSS |
| | | YTGYSTRVFGGGTKLEIK |
| 136 | VL | QSVLTQPASVSGSPGQSITISCAGTSSDVGGNYYVSWYQQHPGKAP |
| | | KLMIYQDSNRPSGVSNRFSGSKSGNTASLTISGLQAEDEADYYCSS |
| | | YTGYSTRVFGGGTKLAVL |
| 137 | VL-CL | QSVLTQPASVSGSPGQSITISCAGTSSDVGGNYYVSWYQQHPGKAP |
| | | KLMIYQDSNRPSGVSNRFSGSKSGNTASLTISGLQAEDEADYYCSS |
| | | YTGYSTRVFGGGTKLEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC |
| 138 | VH-CDR1 | GFTFSNAY |
| 139 | VH-CDR2 | ISGSSSYI |
| 140 | VH-CDR3 | VRSSYYNYDYGDAMDV |
| 141 | VH-FR1 | EVQLVESGGSLVKPGGSLRLSCAAS |
| 142 | VH-FR2 | MNWVRQAPGKGLEWISS |
| 143 | VH-FR3 | GYADFVKGRFTISRDNAKNSLYLQMNSLRAEDTAVYYC |
| 144 | VH-FR4 | WGRGTLVTVSS |
| 145 | VH | EVQLVESGGSLVKPGGSLRLSCAASGFTFSNAYMNWVRQAPGKGLE |
| | | WISSISGSSSYIGYADFVKGRFTISRDNAKNSLYLQMNSLRAEDTA |
| | | VYYCVRSSYYNYDYGDAMDVWGRGTLVTVSS |
| 146 | VH-CH | EVQLVESGGSLVKPGGSLRLSCAASGFTFSNAYMNWVRQAPGKGLE |
| | | WISSISGSSSYIGYADFVKGRFTISRDNAKNSLYLQMNSLRAEDTA |
| | | VYYCVRSSYYNYDYGDAMDVWGRGTLVTVSSASTKGPSVFPLAPSS |
| | | KSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTH |
| | | TCPPCPAPELLGGPSVFLFPPKPKDTLMISRTPEVTCVVVDVSHED |
| | | PEVKFNWYVDGVEVHNAKTKPREEQYNSTYRVVSVLTVLHQDWLNG |
| | | KEYKCKVSNKALPAPIEKTISKAKGQPREPQVYTLPPSRDELTKNQ |
| | | VSLTCLVKGFYPSDIAVEWESNGQPENNYKTTPPVLDSDGSFFLYS |
| | | KLTVDKSRWQQGNVFSCSVMHEALHNHYTQKSLSLSPGK |
| 147 | VH-CH | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |

Monoclonal antibody P3-R5-H03

| | | |
|---|---|---|
| 148 | VL-CDR1 | SSDVGGGYY |
| | VL-CDR2 | GDS |
| 149 | VL-CDR3 | SSNTYYSTRV |
| 150 | VL-FR1 | QSVLTQPASVSGSPGQSITISCAGT |
| 151 | VL-FR2 | VSWYQQHPGKAPKLMIY |
| 152 | VL-FR3 | NRPSGVSNRFSGSKSGNTASLTISGLQAEDEADYYC |
| 153 | VL-FR4 | FGGGTKLEIK |
| 154 | VL-FR4 | FGGGTKLAVL |
| 155 | VL | QSVLTQPASVSGSPGQSITISCAGTSSDVGGGYYVSWYQQHPGKAP |
| | | KLMIYGDSNRPSGVSNRFSGSKSGNTASLTISGLQAEDEADYYCSS |
| | | NTYYSTRVFGGGTKLEIK |
| 156 | VL | QSVLTQPASVSGSPGQSITISCAGTSSDVGGGYYVSWYQQHPGKAP |
| | | KLMIYGDSNRPSGVSNRFSGSKSGNTASLTISGLQAEDEADYYCSS |
| | | NTYYSTRVFGGGTKLAVL |
| 157 | VL-CL | QSVLTQPASVSGSPGQSITISCAGTSSDVGGGYYVSWYQQHPGKAP |
| | | KLMIYGDSNRPSGVSNRFSGSKSGNTASLTISGLQAEDEADYYCSS |
| | | NTYYSTRVFGGGTKLEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC |
| 158 | VH-CDR1 | GFTFSNAG |
| 159 | VH-CDR2 | ISGSSRYI |
| 160 | VH-CDR3 | VRSSSSYGMDV |
| 161 | VH-FR1 | EVQLVESGGSLVKPGGSLRLSCAAS |
| 162 | VH-FR2 | MNWVRQAPGKGLEWISY |
| 163 | VH-FR3 | NYADFVKGRFTISRDNAKNSLYLQMNSLRAEDTAVYYC |
| 164 | VH-FR4 | WGRGTLVTVSS |
| 165 | VH | EVQLVESGGSLVKPGGSLRLSCAASGFTFSNAGMNWVRQAPGKGLE |
| | | WISYISGSSRYINYADFVKGRFTISRDNAKNSLYLQMNSLRAEDTA |
| | | VYYCVRSSSSYGMDVWGRGTLVTVSS |
| 166 | VH-CH | EVQLVESGGSLVKPGGSLRLSCAASGFTFSNAGMNWVRQAPGKGLE |
| | | WISYISGSSRYINYADFVKGRFTISRDNAKNSLYLQMNSLRAEDTA |
| | | VYYCVRSSSSYGMDVWGRGTLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKC |
| | | KVSNKALPAPIEKTISKAKGQPREPQVYTLPPSRDELTKNQVSLTC |
| | | LVKGFYPSDIAVEWESNGQPENNYKTTPPVLDSDGSFFLYSKLTVD |
| | | KSRWQQGNVFSCSVMHEALHNHYTQKSLSLSPGK |
| 167 | VH-CH | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |

Monoclonal antibody P3-R4-F03

| | | |
|---|---|---|
| 168 | VL-CDR1 | SSDVGGSYS |
| | VL-CDR2 | YDS |
| 169 | VL-CDR3 | SSNTQSSTRV |
| 170 | VL-FR1 | QSVLTQPASVSGSPGQSITISCAGT |
| 171 | VL-FR2 | VSWYQQHPGKAPKLMIY |
| 172 | VL-FR3 | YRPSGVSNRFSGSKSGNTASLTISGLQAEDEADYYC |
| 173 | VL-FR4 | FGGGTKLEIK |
| 174 | VL-FR4 | FGGGTKLAVL |
| 175 | VL | QSVLTQPASVSGSPGQSITISCAGTSSDVGGSYSVSWYQQHPGKAP |
| | | KLMIYYDSYRPSGVSNRFSGSKSGNTASLTISGLQAEDEADYYCSS |
| | | NTQSSTRVFGGGTKLEIK |
| 176 | VL | QSVLTQPASVSGSPGQSITISCAGTSSDVGGSYSVSWYQQHPGKAP |
| | | KLMIYYDSYRPSGVSNRFSGSKSGNTASLTISGLQAEDEADYYCSS |
| | | NTQSSTRVFGGGTKLAVL |
| 177 | VL-CL | QSVLTQPASVSGSPGQSITISCAGTSSDVGGSYSVSWYQQHPGKAP |
| | | KLMIYYDSYRPSGVSNRFSGSKSGNTASLTISGLQAEDEADYYCSS |
| | | NTQSSTRVFGGGTKLEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC |
| 178 | VH-CDR1 | GFTFSNNY |
| 179 | VH-CDR2 | IGGSSRDI |
| 180 | VH-CDR3 | VRSNSGMDV |
| 181 | VH-FR1 | EVQLVESGGSLVKPGGSLRLSCAAS |
| 182 | VH-FR2 | MNWVRQAPGKGLEWISS |
| 183 | VH-FR3 | YYADFVKGRFTISRDNAKNSLYLQMNSLRAEDTAVYYC |
| 184 | VH-FR4 | WGRGTLVTVSS |
| 185 | VH | EVQLVESGGSLVKPGGSLRLSCAASGFTFSNNYMNWVRQAPGKGLE |
| | | WISSIGGSSRDIYYADFVKGRFTISRDNAKNSLYLQMNSLRAEDTA |
| | | VYYCVRSNSGMDVWGRGTLVTVSS |
| 186 | VH-CH | EVQLVESGGSLVKPGGSLRLSCAASGFTFSNNYMNWVRQAPGKGLE |
| | | WISSIGGSSRDIYYADFVKGRFTISRDNAKNSLYLQMNSLRAEDTA |
| | | VYYCVRSNSGMDVWGRGTLVTVSSASTKGPSVFPLAPSSKSTSGGT |
| | | AALGCLVKDYFPEPVTVSWNSGALTSGVHTFPAVLQSSGLYSLSSV |
| | | VTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKV |
| | | SNKALPAPIEKTISKAKGQPREPQVYTLPPSRDELTKNQVSLTCLV |
| | | KGFYPSDIAVEWESNGQPENNYKTTPPVLDSDGSFFLYSKLTVDKS |
| | | RWQQGNVFSCSVMHEALHNHYTQKSLSLSPGK |
| 187 | VH-CH | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |

*Nucleotide sequence

1-27. (canceled)

28. An isolated antibody against human TRPV6 channel protein which comprises heavy chain variable CDRs comprising a VH-CDR1 of SEQ ID NO: 158, a VH-CDR2 of SEQ ID NO: 159 and a VH-CDR3 of SEQ ID NO: 160; and light chain variable CDRs comprising a VL-CDR1 of SEQ ID NO: 148, a VL-CDR2 of amino acid sequence GDS and a VL-CDR3 of SEQ ID NO: 149.

29. The isolated antibody according to claim 28, wherein the isolated antibody binds to an extracellular epitope of hTRPV6 protein of SEQ ID NO: 1.

30. The isolated antibody according to claim 28, which binds to an epitope from the third extracellular region of human TRPV6 of SEQ ID NO: 16.

31. The isolated antibody according to claim 28, which modulates the activity of human TRPV6 channel.

32. The isolated antibody according to claim 28, which inhibits the proliferation of TRPV6-expressing cancer cells.

33. The isolated antibody according to claim 28, which is a humanized monoclonal antibody comprising a heavy chain variable domain comprising: a VH-FR1 of SEQ ID NO: 161, a VH-FR2 of SEQ ID NO: 162, a VH-FR3 of SEQ ID NO: 163, and a VH-FR4 of SEQ ID NO: 164; and a light chain variable domain comprising: a VL-FR1 of SEQ ID NO: 150, a VL-FR2 of SEQ ID NO: 151, a VL-FR3 of SEQ ID NO: 152, and a VL-FR4 of SEQ ID NO: 153 or 154.

34. The isolated antibody according to claim 28, which comprises a heavy chain variable domain sequence and a light chain variable domain sequence having at least 90% identity with the pair of sequences SEQ ID NO: 165 and SEQ ID NO: 155 or 156; respectively for the heavy chain variable domain sequence and light chain variable domain sequence.

35. The isolated antibody according to claim 34, which comprises a heavy chain variable domain sequence and a light chain variable domain sequence having the pair of sequences SEQ ID NO: 165 and SEQ ID NO: 155 or 156; respectively for the heavy chain variable domain sequence and light chain variable domain sequence.

36. The isolated antibody according to claim 34, which comprises a heavy chain sequence and a light chain sequence having at least 90% identity with the pair of sequences SEQ ID NO: 166 and SEQ ID NO: 157 respectively for the heavy chain sequence and light chain sequence.

37. The isolated antibody according to claim 36, which comprises a heavy chain sequence and a light chain sequence having the pair of sequences SEQ ID NO: 166 and SEQ ID NO: 157 respectively for the heavy chain sequence and light chain sequence.

38. The isolated antibody according to claim 28, which is a humanized monoclonal antibody of human IgG1 or IgG4 isotype.

39. The isolated antibody according to claim 28, which is coupled to a labeling agent or a therapeutic agent.

40. An expression vector for the recombinant production of the isolated antibody according to claim 28 in a host cell, comprising at least one nucleic acid encoding the heavy and/or light chain of said antibody.

41. A pharmaceutical composition comprising at least an isolated antibody according to claim 28, and a pharmaceutical acceptable vehicle.

42. A method of producing the isolated antibody according to claim 28, comprising culturing a host cell comprising at least one nucleic acid encoding the heavy and/or light chain of said antibody under conditions sufficient for the expression of said antibody.

43. A method for the treatment of cancer associated with TRPV6 expression in a patient in need thereof, said method comprising the administration of a therapeutically effective amount of the isolated antibody according to claim 28 to said patient.

44. A method for in vitro detection of a TRPV6 expression-associated cancer in a biological sample, said method comprising contacting the isolated antibody according claim 28 with the biological sample.

45. The method according to claim 43, wherein the TRPV6 expression associated cancer is selected from the group consisting of: endometrial cancers, leukemia, and cancers of the breast, pancreas, prostate, colon, ovarian, and thyroid.

46. The method according to claim 45 wherein said cancer is prostate cancer.


SEQUENCE LISTING (198 sequences)

SEQ ID NO: 1 - protein, 765 aa, Homo sapiens
MGPLQGDGGPALGGADVAPRLSPVRVWPRPQAPKEPALHPMGLSLPKEKGLILCLWSKFCRWFQRRESWAQSRDEQNLLQQKRIWESPLLLAAKDNDVQALNKLLKYEDCKVHQRGAMGETALHIAALYDNLEAAMVLMEAAPELVFEPMTSELYEGQTALHIAVVNQNMNLVRALLARRASVSARATGTAFRRSPCNLIYFGEHPLSFAACVNSEEIVRLLIEHGADIRAQDSLGNTVLHILILQPNKTFACQMYNLLLSYDRHGDHLQPLDLVPNHQGLTPFKLAGVEGNTVMFQHLMQKRKHTQWTYGPLTSTLYDLTEIDSSGDEQSLLELIITTKKREARQILDQTPVKELVSLKWKRYGRPYFCMLGAIYLLYIICFTMCCIYRPLKPRTNNRTSPRDNTLLQQKLLQEAYMTPKDDIRLVGELVTVIGAIIILLVEVPDIFRMGVTRFFGQTILGGPFHVLIITYAFMVLVTMVMRLISASGEVVPMSFALVLGWCNVMYFARGFQMLGPFTIMIQKMIFGDLMRFCWLMAVVILGFASAFYIIFQTEDPEELGHFYDYPMALFSTFELFLTIIDGPANYNVDLPFMYSITYAAFAIIATLLMLNLLIAMMGDTHWRVAHERDELWRAQIVATTVMLERKLPRCLWPRSGICGREYGLGDRWFLRVEDRQDLNRQRIQRYAQAFHTRGSEDLDKDSVEKLELGCPFSPHLSLPMPSVSRSTSRSSANWERLRQGTLRRDLRGIINRGLEDGESWEYQI

SEQ ID NO: 2 - protein, 17 aa, synthetic construct
  REGION 1..17: note = synthetic peptide (79a-b-c)
LLQEAYMTPKDDIRLVG

SEQ ID NO: 3 - protein, 15 aa, synthetic construct
  REGION 1..15: note = synthetic peptide (79a or P1)
QEAYMTPKDDIRLVG

SEQ ID NO: 4 - protein, 12 aa, synthetic construct
  REGION 1..12: note = synthetic peptide (79b)
QEAYMTPKDDIR

SEQ ID NO: 5 - protein, 14 aa, synthetic construct
  REGION 1..14: note = synthetic peptide (79c)
LLQEAYMTPKDDIR

SEQ ID NO: 6 - protein, 16 aa, synthetic construct
  REGION 1..16: note = synthetic peptide (P1+K)
QEAYMTPKDDIRLVGK

SEQ ID NO: 7 - protein, 12 aa, synthetic construct
  REGION 1..12: note = synthetic peptide (variant)
EAYMTPKEEIRR

SEQ ID NO: 8 - protein, 12 aa, synthetic construct
  REGION 1..12: note = synthetic peptide (wt sequence)
EAYMTPKDDIRL

SEQ ID NO: 9 - protein, 23 aa, synthetic construct
  REGION 1..23: note = synthetic peptide (82a-b-c-d)
IFQTEDPEELGHFYDYPMALFST

SEQ ID NO: 10 - protein, 18 aa, synthetic construct
  REGION 1..18: note = synthetic peptide (82a)
QTEDPEELGHFYDYPMAL

SEQ ID NO: 11 - protein, 17 aa, synthetic construct
  REGION 1..17: note = synthetic peptide (82b)
IFQTEDPEELGHFYDYP

SEQ ID NO: 12 - protein, 17 aa, synthetic construct
  REGION 1..17: note = synthetic peptide (82c)
PEELGHFYDYPMALFST

SEQ ID NO: 13 - protein, 15 aa, synthetic construct
  REGION 1..15: note = synthetic peptide (82d)
TEDPEELGHFYDYPM

SEQ ID NO: 14 - protein, 16 aa, synthetic construct
  REGION 1..16: note = synthetic peptide (P2)
TEDPEELGHFYDYPMA

SEQ ID NO: 15 - protein, 17 aa, synthetic construct
  REGION 1..17: note = synthetic peptide (P2+K)
TEDPEELGHFYDYPMAK

SEQ ID NO: 16 - protein, 15 aa, synthetic construct
  REGION 1..15: note = synthetic peptide (P3)
DGPANYNVDLPFMYS

SEQ ID NO: 17 - protein, 11 aa, synthetic construct
  REGION 1..11: note = synthetic peptide (mAb83 VL-CDR1)
QSLLDSDGRTY

SEQ ID NO: 18 - protein, 9 aa, synthetic construct
  REGION 1..9: note = synthetic peptide (mAb83 VL-CDR3)
WQGTHFPQT

SEQ ID NO: 19 - protein, 26 aa, synthetic construct
  REGION 1..26: note = synthetic peptide (mAb83 VL-FR1)
DVVMTQTPLTLSVTIGQPASISCKSS

SEQ ID NO: 20 - protein, 17 aa, synthetic construct
  REGION 1..17: note = synthetic peptide (mAb83VL-FR2)
LNWLLQRPGQSPKRLIY

SEQ ID NO: 21 - protein, 36 aa, synthetic construct
  REGION 1..36: note = synthetic peptide ( mAb83 VL-FR3)
KLDSGVPDRFTGSGSGTDFTLKISRVEAEDLGVYYC

SEQ ID NO: 22 - protein, 10 aa, synthetic construct
  REGION 1..10: note = synthetic peptide ( mAb83 VL-FR4)
FGGGTKLDIK

SEQ ID NO: 23 - protein, 112 aa, synthetic construct
  REGION 1..112: note = synthetic polypeptide (mAb83 VL)
DVVMTQTPLTLSVTIGQPASISCKSSQSLLDSDGRTYLNWLLQRPGQSPKRLIYLVSKLDSGVPDRFTGSGSGTDFTLKISRVEAEDLGVYYCWQGTHFPQTFGGGTKLDIK

SEQ ID NO: 24 - protein, 219 aa, synthetic construct
  REGION 1..219: note = synthetic polypeptide (mAb83 L chain )
DVVMTQTPLTLSVTIGQPASISCKSSQSLLDSDGRTYLNWLLQRPGQSPKRLIYLVSKLDSGVPDRFTGSGSGTDFTLKISRVEAEDLGVYYCWQGTHFPQTFGGGTKLDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 25 - DNA, 336 bp, synthetic construct
  misc_feature 1..336: note = synthetic polynucleotide (mAb83 VL)
  source 1..336: mol_type = other DNA
gatgttgtgatgacccagactcctctcactttgtcggttaccattggacaaccagcctccatctcttgcaagtcaagtcagagcctcttagatagtgatggaaggacatatttgaattggttgttacagaggccaggccagtctccaaagcgcctaatctatctggtgtctaaactggactctggagtccctgacagattcactggcagtggatcagggacagatttcacactgaaaatcagcagagtggaggctgaggatttgggagtttattattgctggcaaggtacacattttcctcagacgttcggtggaggcaccaagctggacatcaaa

SEQ ID NO: 26 - DNA, 657 bp, synthetic construct
  misc_feature 1..657: note = synthetic polynucleotide (mAb83 L chain)
  source 1..657: mol_type = other DNA
gatgttgtgatgacccagactcctctcactttgtcggttaccattggacaaccagcctccatctcttgcaagtcaagtcagagcctcttagatagtgatggaaggacatatttgaattggttgttacagaggccaggccagtctccaaagcgcctaatctatctggtgtctaaactggactctggagtccctgacagattcactggcagtggatcagggacagatttcacactgaaaatcagcagagtggaggctgaggatttgggagtttattattgctggcaaggtacacattttcctcagacgttcggtggaggcaccaagctggacatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 27 - protein, 8 aa, synthetic construct
  REGION 1..8: note = synthetic peptide (mAb83VH-CDR1)
GFDFSRYW

SEQ ID NO: 28 - protein, 8 aa, synthetic construct
  REGION 1..8: note = synthetic peptide ( mAb83 VH-CDR2)
INPYSSTI

SEQ ID NO: 29 - protein, 8 aa, synthetic construct
  REGION 1..8: note = synthetic peptide ( mAb83 VH-CDR3)
AGKDFFAY

SEQ ID NO: 30 - protein, 25 aa, synthetic construct
  REGION 1..25: note = synthetic peptide (mAb83 VH-FR1)
EVKLIESGGGLVQPGGSLKLSCAAS

SEQ ID NO: 31 - protein, 17 aa, synthetic construct
  REGION 1..17: note = synthetic peptide ( mAb83 VH-FR2)
MSWVRQAPGKGLEWIGE

SEQ ID NO: 32 - protein, 38 aa, synthetic construct
  REGION 1..38: note = synthetic peptide (mAb83 VH-FR3)
NYTPSLKDKFIISRDNAKNTLYLQMRKVRSEDTALYYC

SEQ ID NO: 33 - protein, 11 aa, synthetic construct
  REGION 1..11: note = synthetic peptide ( mAb83 VH-FR4)
WGQGTLVTVSA

SEQ ID NO: 34 - protein, 115 aa, synthetic construct
  REGION 1..115: note = synthetic polypeptide (mAb83 VH)
EVKLIESGGGLVQPGGSLKLSCAASGFDFSRYWMSWVRQAPGKGLEWIGEINPYSSTINYTPSLKDKFIISRDNAKNTLYLQMRKVRSEDTALYYCAGKDFFAYWGQGTLVTVSA

SEQ ID NO: 35 - protein, 439 aa, synthetic construct
  REGION 1..439: note = synthetic polypeptide (mAb83 H chain)
EVKLIESGGGLVQPGGSLKLSCAASGFDFSRYWMSWVRQAPGKGLEWIGEINPYSSTINYTPSLKDKFIISRDNAKNTLYLQMRKVRSEDTALYYCAGKDFFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 36 - DNA, 345 bp, synthetic construct
  misc_feature 1..345: note = synthetic polynucleotide (mAb83 VH)
  source 1..345: mol_type = other DNA
gaggtgaagcttatcgagtctggaggtggcctggtgcagcctggaggatccctgaaactctcctgtgcagcctcaggattcgattttagtagatactggatgagttgggtccggcaggctccagggaaagggctagaatggattggagaaattaatccatatagcagtacgataaactatacgccatctctaaaggataaattcatcatctccagagacaacgccaaaaatacgctgtacctgcaaatgaggaaagtgagatctgaggacacagccctttattactgtgccgggaaggatttttttgcttactggggccaagggactctggtcactgtctctgca

SEQ ID NO: 37 - DNA, 1317 bp, synthetic construct
  misc_feature 1..1317: note = synthetic polynucleotide (mAb83 H chain)
  source 1..1317: mol_type = other DNA
gaggtgaagcttatcgagtctggaggtggcctggtgcagcctggaggatccctgaaactctcctgtgcagcctcaggattcgattttagtagatactggatgagttgggtccggcaggctccagggaaagggctagaatggattggagaaattaatccatatagcagtacgataaactatacgccatctctaaaggataaattcatcatctccagagacaacgccaaaaatacgctgtacctgcaaatgaggaaagtgagatctgaggacacagccctttattactgtgccgggaaggatttttttgcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 38 - protein, 12 aa, synthetic construct
  REGION 1..12: note = synthetic peptide (mAb82 VL-CDR1)
QSLLYSSNQKNY

SEQ ID NO: 39 - protein, 8 aa, synthetic construct
  REGION 1..8: note = synthetic peptide (mAb82 VL-CDR3)
QQYYRYPT

SEQ ID NO: 40 - protein, 26 aa, synthetic construct
  REGION 1..26: note = synthetic peptide (mAb82 VL-FR1)
DIVMSQSPSSLAVSVGEKVTMSCKSS

SEQ ID NO: 41 - protein, 17 aa, synthetic construct
  REGION 1..17: note = synthetic peptide (mAb82 VL-FR2)
LAWYQQKPGQSPKLLIY

SEQ ID NO: 42 - protein, 36 aa, synthetic construct
  REGION 1..36: note = synthetic peptide (mAb82 VL-FR3)
TRESGVPDRFTGSGSGTDFTLTISSVKAEDLAVYYC

SEQ ID NO: 43 - protein, 10 aa, synthetic construct
  REGION 1..10: note = synthetic peptide (mAb82 VL-FR4)
FGGGTKLEIK

SEQ ID NO: 44 - protein, 10 aa, synthetic construct
  REGION 1..10: note = synthetic peptide ( mAb82 VL-FR4 (alternative))
FGGGTKLAVL

SEQ ID NO: 45 - protein, 112 aa, synthetic construct
  REGION 1..112: note = synthetic polypeptide (mAb82 VL)
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYPTFGGGTKLEIK

SEQ ID NO: 46 - protein, 112 aa, synthetic construct
  REGION 1..112: note = synthetic polypeptide (mAb82 VL-alternative)
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYPTFGGGTKLAVL

SEQ ID NO: 47 - protein, 219 aa, synthetic construct
  REGION 1..219: note = synthetic polynucleotide (mAb82 L chain)
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 48 - DNA, 657 bp, synthetic construct
  misc_feature 1..657: note = synthetic polynucleotide (mAb82 L chain)
  source 1..657: mol_type = other DNA
gacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattataggtatccgacgttcggtggaggcaccaagctggaaatcaaacgggctgatgctgcaccaactgtatccatcttcccaccatccagtgagcagttaacatctggaggtgcctcagtcgtgtgcttcttgaacaacttttaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 49 - protein, 8 aa, synthetic construct
  REGION 1..8: note = synthetic peptide (mAb82 VH-CDR1)
GFDFRRYW

SEQ ID NO: 50 - protein, 8 aa, synthetic construct
  REGION 1..8: note = synthetic peptide (mAb82 VH-CDR2)
INPDSSTI

SEQ ID NO: 51 - protein, 11 aa, synthetic construct
  REGION 1..11: note = synthetic peptide (mAb82 VH-CDR3)
ARSASSHYFDY

SEQ ID NO: 52 - protein, 25 aa, synthetic construct
  REGION 1..25: note = synthetic ppetide ( mAb82 VH-FR1)
EVKLLESGGGLVQPGGSLKLSCAAS

SEQ ID NO: 53 - protein, 17 aa, synthetic construct
  REGION 1..17: note = synthetic peptide (mAb82 VH-FR2)
MSWVRQAPGKGLEWIGE

SEQ ID NO: 54 - protein, 38 aa, synthetic construct
  REGION 1..38: note = synthetic peptide (mAb82 VH-FR3)
NYTPSLKDKFIISRDNAKNTLYLQMSKVRSEDTALYYC

SEQ ID NO: 55 - protein, 11 aa, synthetic construct
  REGION 1..11: note = synthetic peptide ( mAb82 VH-FR4)
WGQGTTLTVSS

SEQ ID NO: 56 - protein, 118 aa, synthetic construct
  REGION 1..118: note = synthetic polypeptide (mAb82 VH)
EVKLLESGGGLVQPGGSLKLSCAASGFDFRRYWMSWVRQAPGKGLEWIGEINPDSSTINYTPSLKDKFIISRDNAKNTLYLQMSKVRSEDTALYYCARSASSHYFDYWGQGTTLTVSS

SEQ ID NO: 57 - protein, 448 aa, synthetic construct
  REGION 1..448: note = synthetic polypeptide (mAb82 H chain)
EVKLLESGGGLVQPGGSLKLSCAASGFDFRRYWMSWVRQAPGKGLEWIGEINPDSSTINYTPSLKDKFIISRDNAKNTLYLQMSKVRSEDTALYYCARSASSHYFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 58 - DNA, 1344 bp, synthetic construct
  misc_feature 1..1344: note = synthetic polynucleotide (mAb82 H chain)
  source 1..1344: mol_type = other DNA
gaggtgaagcttctcgagtctggaggtggcctggtgcagcctggaggatccctgaaactctcctgtgcagcctcaggattcgattttagaagatactggatgagttgggtccggcaggctccagggaaagggctagaatggattggagaaattaatccagatagcagtacgataaactatacgccatctctaaaggataaattcatcatctccagagacaacgccaaaaatacgctgtacctgcaaatgagcaaagtgagatctgaggacacagccctttattactgtgcaagatcggcttcatcccactactttgactactggggccaaggcaccactctcacagtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaaaagcttctcccggactccgggtaaa

SEQ ID NO: 59 - protein, 12 aa, synthetic construct
  REGION 1..12: note = synthetic peptide (humanized mAb82 VL-CDR1)
QSLLYSSNQKNY

SEQ ID NO: 60 - protein, 8 aa, synthetic construct
  REGION 1..8: note = synthetic peptide (humanized mAb82 VL-CDR3)
QQYYRYPT

SEQ ID NO: 61 - protein, 26 aa, synthetic construct
  REGION 1..26: note = synthetic peptide (humanized mAb82 VL-FR1)
DIVMSQSPSSLAVSVGEKVTMSCKSS

SEQ ID NO: 62 - protein, 17 aa, synthetic construct
  REGION 1..17: note = synthetic peptide (humanized mAb82 VL-FR2)
LAWYQQKPGQSPKLLIY

SEQ ID NO: 63 - protein, 36 aa, synthetic construct; identical to SEQ ID NO: 42
  REGION 1..36: note = synthetic peptide (humanized mAb82 VL-FR3)

SEQ ID NO: 64 - protein, 10 aa, synthetic construct
  REGION 1..10: note = synthetic peptide (humanized mAb82 VL-FR4)
FGGGTKLEIK

SEQ ID NO: 65 - protein, 10 aa, synthetic construct
  REGION 1..10: note = synthetic peptide (humanized mAb82 VL-FR4 alternative)
FGGGTKLAVL

SEQ ID NO: 66 - protein, 112 aa, synthetic construct; identical to SEQ ID NO: 45
  REGION 1..112: note = synthetic polypeptide (humanized mAb82 VL)

SEQ ID NO: 67 - protein, 112 aa, synthetic construct; identical to SEQ ID NO: 46
  REGION 1..112: note = synthetic polypeptide(humnized mAb82 VL alternative)

SEQ ID NO: 68 - protein, 219 aa, synthetic construct
  REGION 1..219: note = synthetic polypeptide (humanized mAb82 L chain)
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - DNA, 660 bp, synthetic construct
  misc_feature 1..660: note = synthetic polynucleotide (humanized mAb82 L chain)
  source 1..660: mol_type = other DNA
gatatcgttatgactcaaagtccagactcgctggcagtgagccttggggagagagccactataaattgcaagtccagtcaatcactgctgtattcaagcaaccagaaaaactatttggcgtggtatcagcagaagcccggtcagccgcctaaactcctaatttactgggcttctacccgcgaatctggcgtgcctgaccggttttctggatccggctccgggaccgacttcacactcacaatcagctccttacaggccgaagatgtagctgtctattactgtcagcagtactacaggtaccccacgttcggtggaggcaccaaggtggagattaagcgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 70 - protein, 8 aa, synthetic construct
  REGION 1..8: note = synthetic peptide (humanized mAb82 VH-CDR1)
GFDFRRYW

SEQ ID NO: 71 - protein, 8 aa, synthetic construct
  REGION 1..8: note = synthetic peptide (humanized mAb82 VH-CDR2)
INPDSSTI

SEQ ID NO: 72 - protein, 11 aa, synthetic construct
  REGION 1..11: note = synthetic peptide (humanized mAb82 VH-CDR3)
ARSASSHYFDY

SEQ ID NO: 73 - protein, 25 aa, synthetic construct
EVKLLESGGGLVQPGGSLKLSCAAS

SEQ ID NO: 74 - protein, 17 aa, synthetic construct
MSWVRQAPGKGLEWIGE

SEQ ID NO: 75 - protein, 38 aa, synthetic construct; identical to SEQ ID NO: 54
  REGION 1..38: note = synthetic peptide (humanized mAb82 VH-FR3)

SEQ ID NO: 76 - protein, 11 aa, synthetic construct
  REGION 1..11: note = synthetic ppetide (humanized mAb82 VH-FR4)
WGQGTTLTVSS

SEQ ID NO: 77 - protein, 118 aa, synthetic construct; identical to SEQ ID NO: 56
  REGION 1..118: note = synthetic polyppetide (humanized mAb82 VH)

SEQ ID NO: 78 - protein, 448 aa, synthetic construct
  REGION 1..448: note = synthetic polypeptide (humanized mAb82 H chain (IgG1))
EVKLLESGGGLVQPGGSLKLSCAASGFDFRRYWMSWVRQAPGKGLEWIGEINPDSSTINYTPSLKDKFIISRDNAKNTLYLQMSKVRSEDTALYYCARSASSHYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - DNA, 1347 bp, synthetic construct
  misc_feature 1..1347: note = synthetic polynucleotide (humanized mAb82 H chain IgG1)
  source 1..1347: mol_type = other DNA
gaggttcaattagtagaatcgggaggtggtctagttcagccggggggctccctccgtttgtcatgtgcggcttcaggattcgacttccggaggtattggatgcactgggtccggcaggcacccggcaaagggcttgtctgggtgtctaggatcaaccctgacagctccactattaactacactccaagtctgaaagacaagtttataatcagtcgcgataatgccaagaataccctctacctgcagatgtctaaggtgagatccgaggataccgccctgtattactgcgctcgatctgcatccagccattattttgattactggggccagggaacactggtgacagtgagcagcgcaagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 80 - protein, 445 aa, synthetic construct
  REGION 1..445: note = synthetic polypeptide (humanized mAb82 H chain IgG4)
EVKLLESGGGLVQPGGSLKLSCAASGFDFRRYWMSWVRQAPGKGLEWIGEINPDSSTINYTPSLKDKFIISRDNAKNTLYLQMSKVRSEDTALYYCARSASSHYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - DNA, 1338 bp, synthetic construct
  misc_feature 1..1338: note = synthetic polynucleotide (humanized mAb82 H chain IgG4)
  source 1..1338: mol_type = other DNA
gaggttcaattagtagaatcgggaggtggtctagttcagccggggggctccctccgtttgtcatgtgcggcttcaggattcgacttccggaggtattggatgcactgggtccggcaggcacccggcaaagggcttgtctgggtgtctaggatcaaccctgacagctccactattaactacactccaagtctgaaagacaagtttataatcagtcgcgataatgccaagaataccctctacctgcagatgtctaaggtgagatccgaggataccgccctgtattactgcgctcgatctgcatccagccattattttgattactggggccagggaacactggtgacagtgagcagcgcaagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaataa

SEQ ID NO: 82 - protein, 219 aa, synthetic construct
  REGION 1..219: note = synthetic polypeptide (chimeric mAb82 L chain)
DIVMTQSPDSLAVSLGERATINCKSSQSLLYSSNQKNYLAWYQQKPGQPPKLLIYWASTRESGVPDRFSGSGSGTDFTLTISSLQAEDVAVYYCQQYYRYPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - DNA, 660 bp, synthetic construct
  misc_feature 1..660: note = synthetic polynucleotide (chimeric mAb82 L chain)
  source 1..660: mol_type = other DNA
gacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattataggtatccgacgttcggtggaggcaccaagctggaaatcaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 84 - protein, 448 aa, synthetic construct
  REGION 1..448: note = synthetic polypeptide (chimeric mAb82 H chain (IgG1))
EVQLVESGGGLVQPGGSLRLSCAASGFDFRRYWMHWVRQAPGKGLVWVSRINPDSSTINYTPSLKDKFIISRDNAKNTLYLQMSKVRSEDTALYYCARSASSHYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 1347 bp, synthetic construct
  misc_feature 1..1347: note = synthetic polynucleotide (Chimeric mAb82 H chain (IgG1))
  source 1..1347: mol_type = other DNA
gaggtgaagcttctcgagtctggaggtggcctggtgcagcctggaggatccctgaaactctcctgtgcagcctcaggattcgattttagaagatactggatgagttgggtccggcaggctccagggaaagggctagaatggattggagaaattaatccagatagcagtacgataaactatacgccatctctaaaggataaattcatcatctccagagacaacgccaaaaatacgctgtacctgcaaatgagcaaagtgagatctgaggacacagccctttattactgtgcaagatcggcttcatcccactactttgactactggggccaaggcaccactctcacagtctcctcagcaagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 86 - protein, 445 aa, synthetic construct
  REGION 1..445: note = synthetic polypeptide (Chimeric mAb82 H chain (IgG4))
EVQLVESGGGLVQPGGSLRLSCAASGFDFRRYWMHWVRQAPGKGLVWVSRINPDSSTINYTPSLKDKFIISRDNAKNTLYLQMSKVRSEDTALYYCARSASSHYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - DNA, 1338 bp, synthetic construct
  misc_feature 1..1338: note = synthetic polynucleotide (Chimeric mAb82 H chain (IgG4))
  source 1..1338: mol_type = other DNA
gaggtgaagcttctcgagtctggaggtggcctggtgcagcctggaggatccctgaaactctcctgtgcagcctcaggattcgattttagaagatactggatgagttgggtccggcaggctccagggaaagggctagaatggattggagaaattaatccagatagcagtacgataaactatacgccatctctaaaggataaattcatcatctccagagacaacgccaaaaatacgctgtacctgcaaatgagcaaagtgagatctgaggacacagccctttattactgtgcaagatcggcttcatcccactactttgactactggggccaaggcaccactctcacagtctcctcagcaagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaataa

SEQ ID NO: 88 - protein, 9 aa, synthetic construct
  REGION 1..9: note = synthetic peptide (mAb P2-R4-G08 VL-CDR1)
SSDVGGYGQ

SEQ ID NO: 89 - protein, 10 aa, synthetic construct
  REGION 1..10: note = synthetic peptide (mAb P2-R4-G08 VL-CDR3)
SSYTSYSTRV

SEQ ID NO: 90 - protein, 25 aa, synthetic construct
  REGION 1..25: note = synthetic peptide (mAb P2-R4-G08 VL-FR1)
QSVLTQPASVSGSPGQSITISCAGT

SEQ ID NO: 91 - protein, 17 aa, synthetic construct
  REGION 1..17: note = synthetic peptide (mAb P2-R4-G08 VL-FR2)
VSWYQQHPGKAPKLMIY

SEQ ID NO: 92 - protein, 36 aa, synthetic construct
  REGION 1..36: note = synthetic peptide (mAb P2-R4-G08 VL-FR3)
SRPSGVSNRFSGSKSGNTASLTISGLQAEDEADYYC

SEQ ID NO: 93 - protein, 10 aa, synthetic construct
  REGION 1..10: note = synthetic peptide (mAb P2-R4-G08 VL-FR4)
FGGGTKLEIK

SEQ ID NO: 94 - protein, 10 aa, synthetic construct
  REGION 1..10: note = synthetic peptide (mAb P2-R4-G08 VL-FR4 alternative)
FGGGTKLAVL

SEQ ID NO: 95 - protein, 110 aa, synthetic construct
  REGION 1..110: note = synthetic polypeptide (mAb P2-R4-G08 VL)
QSVLTQPASVSGSPGQSITISCAGTSSDVGGYGQVSWYQQHPGKAPKLMIYSDSSRPSGVSNRFSGSKSGNTASLTISGLQAEDEADYYCSSYTSYSTRVFGGGTKLEIK

SEQ ID NO: 96 - protein, 110 aa, synthetic construct
  REGION 1..110: note = synthetic polypeptide ( mAb P2-R4-G08 VL alternative)
QSVLTQPASVSGSPGQSITISCAGTSSDVGGYGQVSWYQQHPGKAPKLMIYSDSSRPSGVSNRFSGSKSGNTASLTISGLQAEDEADYYCSSYTSYSTRVFGGGTKLAVL

SEQ ID NO: 97 - protein, 217 aa, synthetic construct
  REGION 1..217: note = synthetic polypeptide (mAb P2-R4-G08 L chain)
QSVLTQPASVSGSPGQSITISCAGTSSDVGGYGQVSWYQQHPGKAPKLMIYSDSSRPSGVSNRFSGSKSGNTASLTISGLQAEDEADYYCSSYTSYSTRVFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 8 aa, synthetic construct
  REGION 1..8: note = synthetic peptide (mAb P2-R4-G08 VH-CDR1)
GFTFSNYG

SEQ ID NO: 99 - protein, 8 aa, synthetic construct
  REGION 1..8: note = synthetic peptide (mAb P2-R4-G08 VH-CDR2)
ISGSSRSI

SEQ ID NO: 100 - protein, 11 aa, synthetic construct
  REGION 1..11: note = synthetic peptide (mAb P2-R4-G08 VH-CDR3)
VRSSYYYGMDV

SEQ ID NO: 101 - protein, 25 aa, synthetic construct
  REGION 1..25: note = synthetic ppetide (mAb P2-R4-G08 VH-FR1)
EVQLVESGGSLVKPGGSLRLSCAAS

SEQ ID NO: 102 - protein, 17 aa, synthetic construct
  REGION 1..17: note = synthetic peptide ( mAb P2-R4-G08 VH-FR2)
MNWVRQAPGKGLEWISG

SEQ ID NO: 103 - protein, 38 aa, synthetic construct
  REGION 1..38: note = synthetic peptide (mAb P2-R4-G08 VH-FR3)
GYADFVKGRFTISRDNAKNSLYLQMNSLRAEDTAVYYC

SEQ ID NO: 104 - protein, 11 aa, synthetic construct
  REGION 1..11: note = synthetic peptide (mAb P2-R4-G08 VH-FR4)
WGRGTLVTVSS

SEQ ID NO: 105 - protein, 118 aa, synthetic construct
  REGION 1..118: note = synthetic polypeptide (mAb P2-R4-G08 VH)
EVQLVESGGSLVKPGGSLRLSCAASGFTFSNYGMNWVRQAPGKGLEWISGISGSSRSIGYADFVKGRFTISRDNAKNSLYLQMNSLRAEDTAVYYCVRSSYYYGMDVWGRGTLVTVSS

SEQ ID NO: 106 - protein, 448 aa, synthetic construct
  REGION 1..448: note = synthetic polypeptide (mAb P2-R4-G08 H chain)
EVQLVESGGSLVKPGGSLRLSCAASGFTFSNYGMNWVRQAPGKGLEWISGISGSSRSIGYADFVKGRFTISRDNAKNSLYLQMNSLRAEDTAVYYCVRSSYYYGMDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 330 aa, synthetic construct
  REGION 1..330: note = synthetic polypeptide (mAb P2-R4-G08 H chain)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 9 aa, synthetic construct
  REGION 1..9: note = synthetic peptide (mAb P3-R4-E11 VL-CDR1)
SSDVGGYGY

SEQ ID NO: 109 - protein, 10 aa, synthetic construct
  REGION 1..10: note = synthetic peptide (mAb P3-R4-E11 VL-CDR3)
SSYTSQSTRV

SEQ ID NO: 110 - protein, 25 aa, synthetic construct
  REGION 1..25: note = synthetic peptide ( mAb P3-R4-E11 VL-FR1)
QSVLTQPASVSGSPGQSITISCAGT

SEQ ID NO: 111 - protein, 17 aa, synthetic construct
  REGION 1..17: note = synthetic peptide (mAb P3-R4-E11 VL-FR2)
VSWYQQHPGKAPKLMIY

SEQ ID NO: 112 - protein, 36 aa, synthetic construct
  REGION 1..36: note = synthetic peptide (mAb P3-R4-E11 VL-FR3)
YRPSGVSNRFSGSKSGNTASLTISGLQAEDEADYYC

SEQ ID NO: 113 - protein, 10 aa, synthetic construct
  REGION 1..10: note = synthetic peptide (mAb P3-R4-E11 VL-FR4)
FGGGTKLEIK

SEQ ID NO: 114 - protein, 10 aa, synthetic construct
  REGION 1..10: note = synthetic peptide (mAb P3-R4-E11 VL-FR4 alternative)
FGGGTKLAVL

SEQ ID NO: 115 - protein, 110 aa, synthetic construct
  REGION 1..110: note = synthetic polypeptide (mAb P3-R4-E11 VL)
QSVLTQPASVSGSPGQSITISCAGTSSDVGGYGYVSWYQQHPGKAPKLMIYYDSYRPSGVSNRFSGSKSGNTASLTISGLQAEDEADYYCSSYTSQSTRVFGGGTKLEIK

SEQ ID NO: 116 - protein, 110 aa, synthetic construct
  REGION 1..110: note = synthetic peptide (mAb P3-R4-E11 VL alternative)
QSVLTQPASVSGSPGQSITISCAGTSSDVGGYGYVSWYQQHPGKAPKLMIYYDSYRPSGVSNRFSGSKSGNTASLTISGLQAEDEADYYCSSYTSQSTRVFGGGTKLAVL

SEQ ID NO: 117 - protein, 217 aa, synthetic construct
QSVLTQPASVSGSPGQSITISCAGTSSDVGGYGYVSWYQQHPGKAPKLMIYYDSYRPSGVSNRFSGSKSGNTASLTISGLQAEDEADYYCSSYTSQSTRVFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 8 aa, synthetic construct
  REGION 1..8: note = synthetic peptide (mAb P3-R4-E11 VH-CDR1)
GFTFSNSG

SEQ ID NO: 119 - protein, 8 aa, synthetic construct
  REGION 1..8: note = synthetic peptide (mAb P3-R4-E11 VH-CDR2)
ISGSSRYI

SEQ ID NO: 120 - protein, 10 aa, synthetic construct
  REGION 1..10: note = synthetic peptide (mAb P3-R4-E11 VH-CDR3)
VRSNYGGMDV

SEQ ID NO: 121 - protein, 25 aa, synthetic construct
  REGION 1..25: note = synthetic peptide (mAb P3-R4-E11 VH-FR1)
EVQLVESGGSLVKPGGSLRLSCAAS

SEQ ID NO: 122 - protein, 17 aa, synthetic construct
  REGION 1..17: note = synthetic peptide (mAb P3-R4-E11 VH-FR2)
MNWVRQAPGKGLEWISG

SEQ ID NO: 123 - protein, 38 aa, synthetic construct; identical to SEQ ID NO: 103
  REGION 1..38: note = synthetic peptide (mAb P3-R4-E11 VH-FR3)

SEQ ID NO: 124 - protein, 11 aa, synthetic construct
  REGION 1..11: note = synthetic peptide (mAb P3-R4-E11 VH-FR4)
WGRGTLVTVSS

SEQ ID NO: 125 - protein, 117 aa, synthetic construct
  REGION 1..117: note = synthetic polypeptide (mAb P3-R4-E11 VH)
EVQLVESGGSLVKPGGSLRLSCAASGFTFSNSGMNWVRQAPGKGLEWISGISGSSRYIGYADFVKGRFTISRDNAKNSLYLQMNSLRAEDTAVYYCVRSNYGGMDVWGRGTLVTVSS

SEQ ID NO: 126 - protein, 446 aa, synthetic construct
  REGION 1..446: note = synthetic polypeptide (mAb P3-R4-E11 H chain)
EVQLVESGGSLVKPGGSLRLSCAASGFTFSNSGMNWVRQAPGKGLEWISGISGSSRYIGYADFVKGRFTISRDNAKNSLYLQMNSLRAEDTAVYYCVRSNYGGMDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 330 aa, synthetic construct; identical to SEQ ID NO: 107
  REGION 1..330: note = synthetic polypeptide (mAb P3-R4-E11 H chain)

SEQ ID NO: 128 - protein, 9 aa, synthetic construct
  REGION 1..9: note = synthetic peptide (mAb P3-R5-E06 VL-CDR1)
SSDVGGNYY

SEQ ID NO: 129 - protein, 10 aa, synthetic construct
  REGION 1..10: note = synthetic peptide (mAb P3-R5-E06 VL-CDR3)
SSYTGYSTRV

SEQ ID NO: 130 - protein, 25 aa, synthetic construct
  REGION 1..25: note = synthetic peptide (mAb P3-R5-E06 VL-FR1)
QSVLTQPASVSGSPGQSITISCAGT

SEQ ID NO: 131 - protein, 17 aa, synthetic construct
  REGION 1..17: note = synthetic peptide (mAb P3-R5-E06 VL-FR2)
VSWYQQHPGKAPKLMIY

SEQ ID NO: 132 - protein, 36 aa, synthetic construct
  REGION 1..36: note = synthetic peptide (mAb P3-R5-E06 VL-FR3)
NRPSGVSNRFSGSKSGNTASLTISGLQAEDEADYYC

SEQ ID NO: 133 - protein, 10 aa, synthetic construct
  REGION 1..10: note = synthetic peptide (mAb P3-R5-E06 VL-FR4)
FGGGTKLEIK

SEQ ID NO: 134 - protein, 10 aa, synthetic construct
  REGION 1..10: note = synthetic peptide (mAb P3-R5-E06 VL-FR4 alternative)
FGGGTKLAVL

SEQ ID NO: 135 - protein, 110 aa, synthetic construct
  REGION 1..110: note = synthetic polypeptide (mAb P3-R5-E06 VL)
QSVLTQPASVSGSPGQSITISCAGTSSDVGGNYYVSWYQQHPGKAPKLMIYQDSNRPSGVSNRFSGSKSGNTASLTISGLQAEDEADYYCSSYTGYSTRVFGGGTKLEIK

SEQ ID NO: 136 - protein, 110 aa, synthetic construct
  REGION 1..110: note = synthetic polypeptide (mAb P3-R5-E06 VL alternative)
QSVLTQPASVSGSPGQSITISCAGTSSDVGGNYYVSWYQQHPGKAPKLMIYQDSNRPSGVSNRFSGSKSGNTASLTISGLQAEDEADYYCSSYTGYSTRVFGGGTKLAVL

SEQ ID NO: 137 - protein, 217 aa, synthetic construct
  REGION 1..217: note = synthetic polypeptide (mAb P3-R5-E06 L chain)
QSVLTQPASVSGSPGQSITISCAGTSSDVGGNYYVSWYQQHPGKAPKLMIYQDSNRPSGVSNRFSGSKSGNTASLTISGLQAEDEADYYCSSYTGYSTRVFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 8 aa, synthetic construct
  REGION 1..8: note = synthetic peptide (mAb P3-R5-E06 VH-CDR1)
GFTFSNAY

SEQ ID NO: 139 - protein, 8 aa, synthetic construct
  REGION 1..8: note = synthetic peptide (mAb P3-R5-E06 VH-CDR2)
ISGSSSYI

SEQ ID NO: 140 - protein, 16 aa, synthetic construct
  REGION 1..16: note = synthetic peptide (mAb P3-R5-E06 VH-CDR3)
VRSSYYNYDYGDAMDV

SEQ ID NO: 141 - protein, 25 aa, synthetic construct
  REGION 1..25: note = synthetic peptide (mAb P3-R5-E06 VH-FR1)
EVQLVESGGSLVKPGGSLRLSCAAS

SEQ ID NO: 142 - protein, 17 aa, synthetic construct
  REGION 1..17: note = synthetic peptide (mAb P3-R5-E06 VH-FR2)
MNWVRQAPGKGLEWISS

SEQ ID NO: 143 - protein, 38 aa, synthetic construct; identical to SEQ ID NO: 103
  REGION 1..38: note = synthetic peptide (mAb P3-R5-E06 VH-FR3)

SEQ ID NO: 144 - protein, 11 aa, synthetic construct
  REGION 1..11: note = synthetic peptide (mAb P3-R5-E06 VH-FR4)
WGRGTLVTVSS

SEQ ID NO: 145 - protein, 123 aa, synthetic construct
  REGION 1..123: note = synthetic peptide (mAb P3-R5-E06 VH)
EVQLVESGGSLVKPGGSLRLSCAASGFTFSNAYMNWVRQAPGKGLEWISSISGSSSYIGYADFVKGRFTISRDNAKNSLYLQMNSLRAEDTAVYYCVRSSYYNYDYGDAMDVWGRGTLVTVSS

SEQ ID NO: 146 - protein, 453 aa, synthetic construct
  REGION 1..453: note = synthetic peptide (mAb P3-R5-E06 H chain)
EVQLVESGGSLVKPGGSLRLSCAASGFTFSNAYMNWVRQAPGKGLEWISSISGSSSYIGYADFVKGRFTISRDNAKNSLYLQMNSLRAEDTAVYYCVRSSYYNYDYGDAMDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 330 aa, synthetic construct; identical to SEQ ID NO: 107
  REGION 1..330: note = synthetic peptide (mAb P3-R5-E06 H chain)

SEQ ID NO: 148 - protein, 9 aa, synthetic construct
  REGION 1..9: note = synthetic peptide(mAb P3-R5-H03 VL-CDR1)
SSDVGGGYY

SEQ ID NO: 149 - protein, 10 aa, synthetic construct
  REGION 1..10: note = synthetic peptide (mAb P3-R5-H03 VL-CDR3)
SSNTYYSTRV

SEQ ID NO: 150 - protein, 25 aa, synthetic construct
  REGION 1..25: note = synthetic peptide (mAb P3-R5-H03 VL-FR1)
QSVLTQPASVSGSPGQSITISCAGT

SEQ ID NO: 151 - protein, 17 aa, synthetic construct
  REGION 1..17: note = synthetic peptide (mAb P3-R5-H03 VL-FR2)
VSWYQQHPGKAPKLMIY

SEQ ID NO: 152 - protein, 36 aa, synthetic construct; identical to SEQ ID NO: 132
  REGION 1..36: note = synthetic peptide (mAb P3-R5-H03 VL-FR3)

SEQ ID NO: 153 - protein, 10 aa, synthetic construct
  REGION 1..10: note = synthetic peptide (mAb P3-R5-H03 VL-FR4)
FGGGTKLEIK

SEQ ID NO: 154 - protein, 10 aa, synthetic construct
  REGION 1..10: note = synthetic peptide (mAb P3-R5-H03 VL-FR4 alternative)
FGGGTKLAVL

SEQ ID NO: 155 - protein, 110 aa, synthetic construct
  REGION 1..110: note = synthetic polypeptide (mAb P3-R5-H03 VL)
QSVLTQPASVSGSPGQSITISCAGTSSDVGGGYYVSWYQQHPGKAPKLMIYGDSNRPSGVSNRFSGSKSGNTASLTISGLQAEDEADYYCSSNTYYSTRVFGGGTKLEIK

SEQ ID NO: 156 - protein, 110 aa, synthetic construct
  REGION 1..110: note = synthetic polypeptide (mAb P3-R5-H03 VL alternative)
QSVLTQPASVSGSPGQSITISCAGTSSDVGGGYYVSWYQQHPGKAPKLMIYGDSNRPSGVSNRFSGSKSGNTASLTISGLQAEDEADYYCSSNTYYSTRVFGGGTKLAVL

SEQ ID NO: 157 - protein, 217 aa, synthetic construct
  REGION 1..217: note = synthetic polypeptide (mAb P3-R5-H03 L chain)
QSVLTQPASVSGSPGQSITISCAGTSSDVGGGYYVSWYQQHPGKAPKLMIYGDSNRPSGVSNRFSGSKSGNTASLTISGLQAEDEADYYCSSNTYYSTRVFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 8 aa, synthetic construct
  REGION 1..8: note = synthetic peptide (mAb P3-R5-H03 VH-CDR1)
GFTFSNAG

SEQ ID NO: 159 - protein, 8 aa, synthetic construct
  REGION 1..8: note = synthetic peptide (mAb P3-R5-H03 VH-CDR2)
ISGSSRYI

SEQ ID NO: 160 - protein, 11 aa, synthetic construct
  REGION 1..11: note = synthetic peptide (mAb P3-R5-H03 VH-CDR3)
VRSSSSYGMDV

SEQ ID NO: 161 - protein, 25 aa, synthetic construct
  REGION 1..25: note = synthetic peptide (mAb P3-R5-H03 VH-FR1)
EVQLVESGGSLVKPGGSLRLSCAAS

SEQ ID NO: 162 - protein, 17 aa, synthetic construct
  REGION 1..17: note = synthetic peptide (mAb P3-R5-H03 VH-FR2)
MNWVRQAPGKGLEWISY

SEQ ID NO: 163 - protein, 38 aa, synthetic construct
  REGION 1..38: note = synthetic peptide (mAb P3-R5-H03 VH-FR3)
NYADFVKGRFTISRDNAKNSLYLQMNSLRAEDTAVYYC

SEQ ID NO: 164 - protein, 11 aa, synthetic construct
  REGION 1..11: note = synthetic peptide (mAb P3-R5-H03 VH-FR4)
WGRGTLVTVSS

SEQ ID NO: 165 - protein, 118 aa, synthetic construct
  REGION 1..118: note = synthetic polypeptide (mAb P3-R5-H03 VH)
EVQLVESGGSLVKPGGSLRLSCAASGFTFSNAGMNWVRQAPGKGLEWISYISGSSRYINYADFVKGRFTISRDNAKNSLYLQMNSLRAEDTAVYYCVRSSSSYGMDVWGRGTLVTVSS

SEQ ID NO: 166 - protein, 448 aa, synthetic construct
  REGION 1..448: note = synthetic polypeptide (mAb P3-R5-H03 H chain)
EVQLVESGGSLVKPGGSLRLSCAASGFTFSNAGMNWVRQAPGKGLEWISYISGSSRYINYADFVKGRFTISRDNAKNSLYLQMNSLRAEDTAVYYCVRSSSSYGMDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 330 aa, synthetic construct; identical to SEQ ID NO: 107
  REGION 1..330: note = synthetic polypeptide (mAb P3-R5-H03 H chain)

SEQ ID NO: 168 - protein, 9 aa, synthetic construct
  REGION 1..9: note = synthetic peptide (mAb P3-R4-F03 VL-CDR1)
SSDVGGSYS

SEQ ID NO: 169 - protein, 10 aa, synthetic construct
  REGION 1..10: note = synthetic peptide (mAb P3-R4-F03 VL-CDR3)
SSNTQSSTRV

SEQ ID NO: 170 - protein, 25 aa, synthetic construct
  REGION 1..25: note = synthetic peptide (mAb P3-R4-F03 VL-FR1)
QSVLTQPASVSGSPGQSITISCAGT

SEQ ID NO: 171 - protein, 17 aa, synthetic construct
  REGION 1..17: note = synthetic peptide (mAb P3-R4-F03 VL-FR2)
VSWYQQHPGKAPKLMIY

SEQ ID NO: 172 - protein, 36 aa, synthetic construct; identical to SEQ ID NO: 112
  REGION 1..36: note = synthetic peptide (mAb P3-R4-F03 VL-FR3)

SEQ ID NO: 173 - protein, 10 aa, synthetic construct
  REGION 1..10: note = synthetic peptide (mAb P3-R4-F03 VL-FR4)
FGGGTKLEIK

SEQ ID NO: 174 - protein, 10 aa, synthetic construct
FGGGTKLAVL

SEQ ID NO: 175 - protein, 110 aa, synthetic construct
  REGION 1..110: note = synthetic polypeptide (mAb P3-R4-F03 VL)
QSVLTQPASVSGSPGQSITISCAGTSSDVGGSYSVSWYQQHPGKAPKLMIYYDSYRPSGVSNRFSGSKSGNTASLTISGLQAEDEADYYCSSNTQSSTRVFGGGTKLEIK

SEQ ID NO: 176 - protein, 110 aa, synthetic construct
  REGION 1..110: note = synthetic polypeptide (mAb P3-R4-F03 VL alternative)
QSVLTQPASVSGSPGQSITISCAGTSSDVGGSYSVSWYQQHPGKAPKLMIYYDSYRPSGVSNRFSGSKSGNTASLTISGLQAEDEADYYCSSNTQSSTRVFGGGTKLAVL

SEQ ID NO: 177 - protein, 217 aa, synthetic construct
  REGION 1..217: note = synthetic polypeptide (mAb P3-R4-F03 L chain)
QSVLTQPASVSGSPGQSITISCAGTSSDVGGSYSVSWYQQHPGKAPKLMIYYDSYRPSGVSNRFSGSKSGNTASLTISGLQAEDEADYYCSSNTQSSTRVFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 8 aa, synthetic construct
  REGION 1..8: note = synthetic peptide (mAb P3-R4-F03 VH-CDR1)
GFTFSNNY

SEQ ID NO: 179 - protein, 8 aa, synthetic construct
  REGION 1..8: note = synthetic peptide (mAb P3-R4-F03 VH-CDR2)
IGGSSRDI

SEQ ID NO: 180 - protein, 9 aa, synthetic construct
  REGION 1..9: note = synthetic peptide (mAb P3-R4-F03 VH-CDR3)
VRSNSGMDV

SEQ ID NO: 181 - protein, 25 aa, synthetic construct
  REGION 1..25: note = synthetic peptide (mAb P3-R4-F03 VH-FR1)
EVQLVESGGSLVKPGGSLRLSCAAS

SEQ ID NO: 182 - protein, 17 aa, synthetic construct
  REGION 1..17: note = synthetic peptide (mAb P3-R4-F03 VH-FR2)
MNWVRQAPGKGLEWISS

SEQ ID NO: 183 - protein, 38 aa, synthetic construct
  REGION 1..38: note = synthetic peptide (mAb P3-R4-F03 VH-FR3)
YYADFVKGRFTISRDNAKNSLYLQMNSLRAEDTAVYYC

SEQ ID NO: 184 - protein, 11 aa, synthetic construct
  REGION 1..11: note = synthetic peptide (mAb P3-R4-F03 VH-FR4)
WGRGTLVTVSS

SEQ ID NO: 185 - protein, 116 aa, synthetic construct
  REGION 1..116: note = synthetic polynucleotide (mAb P3-R4-F03 VH)
EVQLVESGGSLVKPGGSLRLSCAASGFTFSNNYMNWVRQAPGKGLEWISSIGGSSRDIYYADFVKGRFTISRDNAKNSLYLQMNSLRAEDTAVYYCVRSNSGMDVWGRGTLVTVSS

SEQ ID NO: 186 - protein, 446 aa, synthetic construct
  REGION 1..446: note = synthetic polypeptide (mAb P3-R4-F03 H chain)
EVQLVESGGSLVKPGGSLRLSCAASGFTFSNNYMNWVRQAPGKGLEWISSIGGSSRDIYYADFVKGRFTISRDNAKNSLYLQMNSLRAEDTAVYYCVRSNSGMDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 330 aa, synthetic construct; identical to SEQ ID NO: 107
  REGION 1..330: note = synthetic polypeptide (mAb P3-R4-F03 H chain)

SEQ ID NO: 188 - protein, 15 aa, synthetic construct
  REGION 1..15: note = synthetic peptide
QRRESWAQSRDEQNL

SEQ ID NO: 189 - protein, 16 aa, synthetic construct
  REGION 1..16: note = synthetic peptide
HTRGSEDLDKDSVEKL

SEQ ID NO: 190 - DNA, 20 bp, synthetic construct
  misc_feature 1..20: note = synthetic oligonucleotide
  source 1..20: mol_type = other DNA
cccaaggagaaagggctaat

SEQ ID NO: 191 - DNA, 20 bp, synthetic construct
  misc_feature 1..20: note = synthetic oligonucleotide
  source 1..20: mol_type = other DNA
ttggcagctagaaggagagg

SEQ ID NO: 192 - DNA, 21 bp, synthetic construct
  misc_feature 1..21: note = synthetic oligonucleotide
  source 1..21: mol_type = other DNA
ggcgtcgtgattagtgatgat

SEQ ID NO: 193 - DNA, 20 bp, synthetic construct
  misc_feature 1..20: note = synthetic oligonucleotide
  source 1..20: mol_type = other DNA
cgagcaagacgttcagtcct

SEQ ID NO: 194 - RNA, 19 nt, synthetic construct
  misc_feature 1..19: note = synthetic oligonucleotide (siRNA 3'dTdT)
  source 1..19: mol_type = other RNA
cctgctgcagcagaagagg

SEQ ID NO: 195 - RNA, 19 nt, synthetic construct
  misc_feature 1..19: note = synthetic oligonucleotide (siRNA 3'dTdT)
  source 1..19: mol_type = other RNA
gactctctatgacctcaca

SEQ ID NO: 196 - RNA, 19 nt, synthetic construct
  misc_feature 1..19: note = synthetic oligonucleotide (siRNA 3'dTdT)
  source 1..19: mol_type = other RNA
cgtcatgtacttcgcccga

SEQ ID NO: 197 - RNA, 19 nt, synthetic construct
  misc_feature 1..19: note = synthetic oligonucleotide (siRNA 3'dTdT)
  source 1..19: mol_type = other RNA
cctcctcattgccatgatg

SEQ ID NO: 198 - RNA, 20 nt, synthetic construct
  misc_feature 1..20: note = synthetic oligonucleotide (siRNA 3'dTdT)
  source 1..20: mol_type = other RNA
cttacgcctgagtacttcga